(19) United States
(12) Patent Application Publication
(10) Pub. No.: US 2021/0214681 A1
(43) Pub. Date: Jul. 15, 2021

(54) STEM CELL-DERIVED HUMAN MICROGLIAL CELLS, METHODS OF MAKING AND METHODS OF USE

(71) Applicant: MEMORIAL SLOAN-KETTERING CANCER CENTER, New York, NY (US)

(72) Inventors: Lorenz Studer, New York, NY (US); Sudha Ragavalli Guttikonda, New York, NY (US)

(73) Assignee: MEMORIAL SLOAN-KETTERING CANCER CENTER, New York, NY (US)

(21) Appl. No.: 17/213,830

(22) Filed: Mar. 26, 2021

Related U.S. Application Data
(63) Continuation of application No. PCT/US2019/053852, filed on Sep. 30, 2019.
(60) Provisional application No. 62/738,176, filed on Sep. 28, 2018.

(51) Int. Cl. C12N 5/079; A61P 25/28; A61P 25/16; A61K 35/30; G01N 33/50

(52) U.S. Cl. CPC C12N 5/0622; C12N 2506/45; A61P 25/16; A61K 35/30; G01N 33/5041; G01N 33/5058; C12N 2501/415; C12N 2501/165; C12N 2501/115; C12N 2501/125; C12N 2501/145; C12N 2501/22; C12N 2501/2334; C12N 2501/2303; C12N 2501/105; C12N 2501/155; C12N 2501/16; C12N 2501/2306; C12N 2502/081; A61P 25/28

(57) ABSTRACT

The present disclosure relates to methods for generating microglial cells derived from stem cells (e.g., human stem cells), microglial cells obtained from such methods and compositions comprising thereof, and uses of said microglial cells for disease modeling and for treating microglia related disorders.

### CROSS-REFERENCE TO RELATED APPLICATIONS

This application is a Continuation of International Patent Application No. PCT/US2019/053852, filed Sep. 30, 2019, which claims priority to U.S. Provisional Application No. 62/738,176, filed Sep. 28, 2018, the contents of each which are hereby incorporated by reference in their entirety , and to each of which priority is claimed.

### 1. INTRODUCTION

The present disclosure relates to methods for generating microglial cells derived from stem cells (e.g., human stem cells), microglial cells obtained from such methods and compositions comprising thereof, uses of such microglial cells for disease modeling and for treating microglia related disorders.

### 2. BACKGROUND

Microglia (i.e., microglial cells) are the tissue-resident macrophages of the central nervous system (CNS), colonizing the brain during early embryonic development (Prinz et al., Nat. Rev. Neurosci. (2014); 15, 300-312). These phagocytic cells are implicated in many roles in the central nervous system: including clearance of cellular debris, signaling to neurons and astrocytes to mediate synaptic connectivity, and maintenance of CNS homeostasis. Most current literature on microglial biology has only been explored in rodent models (Béchade et al., Front Cell Neurosci. (2013); 7, 32; Bialas et al., Nat. Neurosci. (2013); 16, 1773-1782; Bilimoria et al., Brain Res. (2015); 1617, 7-17; Ji et al., PLoS One (2013); 8; 48; Pascual et al., Proc. Natl. Acad. Sci. (2012);109; Schafer et al., Neuron (2012); 74, 691-705; Tremblay et al. PLoS Biol. (2010); 8; Wu et al., Trends Immunol. (2015); 36, 605-613; Zhan et al. Nat. Neurosci. (2014); 17, 400-406).

While the mouse has been a powerful mammalian system to show parallels with human pathology, there are significant differences between mouse and human microglia (Zhang et al., Neuron (2016); 89, 37-53). These include fundamental receptors that are only expressed in mouse but not human, such as the macrophage F4/80 receptor, as well as functional differences in response to drug treatments (Smith et al., Trends Neurosci. (2014); 37, 125-135). In addition, certain neurodevelopmental diseases are strongly associated with genes that are not expressed in the mouse, such as schizophrenia and the complement component C4A allele (Havik et al., Biol. Psychiatry (2012); 70, 35-42; Sekar et al., Nature (2016);530, 177-183). However, human primary microglia are expensive, hard to obtain from post-mortem tissue, and do not proliferate in culture, making disease modeling extremely difficult (Melief et al., Glia. (2016); 64(11):1857-681). The alternative, human immortalized microglial cell lines, do not share key genes with primary microglia (Melief et al., Glia. (2016); 64(11):1857-681).

Human pluripotent stem cells (i.e., hPSCs) derived microglia allow for a supply of developmentally derived microglia from patient cells that have the same function as microglia found in the patient brain. hPSCs are self-renewable cells from either human embryonic stem cells (i.e., ES cells) or induced-pluripotent stem cells (i.e., iPS cells) that have the ability to differentiate into any cell type in the body. Typically, iPS cells are derived from somatic cells of individuals suffering from a particular disease. hPSC-derived microglia can be used to explore non-cell autonomous interactions between neurons and microglia when modeling diseases, and they can be used to screen potential drug targets (Hoing et al., Cell Stem Cell (2012);11, 620-632). Moreover, hPSC-derived microglia can be used as potential cell-therapy for neurodevelopmental and neurodegenerative diseases, as well as CNS brain tumors.

Microglia are the only tissue-resident macrophages that directly develop from yolk sac precursors, whereas others develop from precursors which migrate to the liver first (Hoeffel et al., Immunity (2015); 42, 665-678). Therefore, it is important to model early yolk sac primitive hematopoiesis to generate microglia.

Existing strategies to generate human microglia in vitro do not follow this developmental paradigm, failing to first initiate primitive hematopoiesis. These strategies either start from peripheral monocytes (Noto et al., Neuropathol. Appl. Neurobiol. (2014); 40, 697-713; Ohgidani et al., Sci. Rep. (2014); 4, 4957), which arise from definitive hematopoiesis and therefore do not give rise to microglia in vivo, or by using embryoid body approaches which are not pre-patterned for primitive hematopoiesis and therefore are black boxes with respect to developmental cell fate decisions (Etemad et al., Neurosci. (2012); 209, 79-89; Hinze et al., Inflamm. (2012); 9, 12; Lachmann et al., Stem Cell Reports (2015);4, 282-296; Noto et al., Neuropathol. Appl. Neurobiol. (2014); 40, 697-713; Ohgidani et al., Sci. Rep. (2014); 4, 4957; van Wilgenburg et al., PLoS One (2013); 8; Muffat et al., Nature Medicine (2016); 22(11), 1358-1367; Bahrini et al., Sci. Rep. (2015); 5, 7989). It is difficult to determine if these strategies go through early erythromyeloid precursors (EMPs), which are the true precursors to microglia, or late EMPs, which give rise to definitive hematopoiesis. Thus, there is a need for a novel method for generating human microglial cells.

### 3. BRIEF DESCRIPTION OF THE DRAWINGS

FIGS. 1A and 1B depict differentiation methods in accordance with certain embodiments of the present disclosure. FIG. 1A shows combination of Wnt inhibition and activation yielded KDR⁺CD235a⁺ double positive primitive hematopoietic precursors. Eighteen hours of Wnt activation by a Wnt agonist Chir099021 gave rise to early mesoderm, marked by Brachyury (T) by immunofluorescence. These cells were also KDR+ and CD235a+ (primitive hematopoietic precursors) by day 4 of differentiation by flow cytometry. FIG. 1B shows timing of ChiR exposure. The concentration and timing of Chir099021 exposure in the differentiation scheme was optimized, where exposure for 18 hr at 3 uM followed by Wnt inhibition via IWP2 at 3 uM yielded the highest percentage of primitive hematopoietic precursors (KDR+CD235a+) by flow cytometry.

FIG. 2 shows timeline of hemogenic endothelium and hematopoietic cell development. Hemogenic endothelium developed after 1 day of replating the sorted KDR+CD235a+ primitive hematopoietic precursors. These cells gave rise to hematopoietic cells in suspension over 7 days in culture, gradually becoming more numerous in culture.

FIG. 3 shows validation of hemogenic endothelium by VE-cadherin+ staining. At 1 day post-sort, the cells were positive for VE-cadherin by immunofluorescence confirming that they were hemogenic endothelium, and the cells in suspension had characteristic small nuclei of hematopoietic cells.

FIG. 4 shows round cell cultures proceeded through developmental intermediates: Kit+, Kit+CD45+. At day 5 after replating the primitive hematopoietic precursors, Kit+ cells emerged, which were early erythromyeloid progenitors (EMPs), the precursors to microglia. These cells then gained CD45+ by flow cytometry, indicating complete hematopoietic commitment.

FIG. 5 shows strategies for deriving hPSC-microglia. From the EMP stage, microglia can be derived either through direct co-culture with neurons for at least 4 days, or matured to macrophages separately, and then co-cultured with neurons for at least 11 days.

FIG. 6 shows macrophage markers present on a small subset of EMPs/pMacs (round cells, precursors). In day 8 post sort cultures, where there was a mixed population of EMPs, these cells were identified as microglial and macrophage progenitors and not mature macrophages. Only a small proportion of macrophages expressed mature macrophage markers such as CX3CR1, CD14, Cd11b, and MRC. Most cells expressed CD45, however, indicating that they were committed to the hematopoietic lineage. All markers were checked by flow cytometry.

FIG. 7 shows culturing pMacs with neurons gave rise to Iba1⁺ Pu.1⁺ cells by day 4 of co-culture. Culturing the cells in suspension at day 8 post-sort, which contained pMacs, with neurons gave rise to early microglial cells by only day 4 of co-culture. These cells were identified with Iba1+ and Pu.1+ staining. All cells that were CD45⁺ were also Pu.1+, indicating that any hematopoietic cell that had persisted in culture (delineated by CD45+), was committed to the myeloid/microglial lineage (delineated by PU.1). This indicates that this strategy is highly efficient, by immunofluorescence.

FIG. 8 shows Iba1⁺ cells were stable in culture. The microglial cells (Iba1⁺ by immunofluorescence) were still persistent in co-culture after 14 days of co-culture with cortical neurons.

FIG. 9 shows Iba1⁺ cells were stable in culture. The microglial cells (Iba1⁺, PU.1⁺ by immunofluorescence) still persisted in co-culture with neurons after 21 days in culture.

FIG. 10 shows culture pMacs in RPMI+10% serum, M-CSF, IL-34. In the second strategy to generate microglia, the EMPs were matured into macrophages with 10% serum and M-CSF and IL-34. They developed the mature macrophage marker CD11b by day 4 in culture, and the mature spindle morphology by day 11.

FIG. 11 shows efficient induction of macrophages detected using macrophage markers present on majority of cells after 11 days indicating mature macrophages. All the cells that persisted in culture in serum and M-CSF and IL-34 after 11 days were CD45⁺, indicating they were all hematopoietic. The majority also were positive for mature macrophage markers such as CX3CR1, CD14, CD11b, and Dectin by flow cytometry analysis.

FIG. 12 provides serum free method for the culture pMacs in IMDM/F12/N2/B27+M-CSF, IL-34. The pMacs can also be matured into macrophages without serum, using IMDM/F12/N2/B27 in place of serum along with M-CSF and IL-34. Addition of M-CSF with IL-34 increased yield by encouraging cell division. Addition of GM-CSF allowed division of the cells as well but the resulting cells appeared more granular and activated.

FIG. 13 shows that co-culture of macrophage-like cells with neurons showed strong Iba1 and Pu.1 expression. Macrophage cells co-cultured with neurons yielded microglial cells with branching morphology and increased Iba1 immunofluorescence staining. Iba1 was increased in microglial cells from macrophages, so the cells had transitioned into microglial identity through co-culture with neurons.

FIG. 14 shows that co-cultured pMac-derived microglia (EMP co-culture) and pMac-macrophage derived microglia (EMP-macrophage co-culture) share expression of key microglial genes with human fetal microglia. Quantitative PCR of RNA from the two different strategies to derive microglia shared gene expression of a panel of key microglial genes with RNA from human fetal microglia (commercial source). In contrast, monocyte-derived macrophages, which represented peripheral macrophages, did not express these markers. Noticeably, when the EMP-derived macrophages were cultured alone, they down regulated the key microglial genes TMEM119 and SALL1, indicating that co-culture was necessary to maintain microglial identity.

FIG. 15 shows day 14 tri-culture of neurons, microglia and astrocytes. The microglial cells that derived from the present disclosure can be co-cultured with astrocytes to build a system containing three components of the CNS: neurons, microglia, and astrocytes. Microglia were tagged in RFP, astrocytes were GFAP⁺ by immunofluorescence.

FIG. 16 shows day 30 tri-culture of neurons, microglia and astrocytes. The microglial cells that derived from the present disclosure can be co-cultured with astrocytes to build a system containing three components of the CNS: neurons, microglia, and astrocytes. Microglia were tagged in RFP, astrocytes were GFAP+ by immunofluorescence.

FIG. 17 shows tri-culture system that can be used to study interactions between cell types. Inflammatory stimuli, or a disease state inducing an inflammatory stimulus, affects both microglia and astrocytes which have crosstalk. This crosstalk is a feedback or feedforward loop that can then lead to toxicity to neurons. This interaction can be studied using the presently disclosed hPSC-derived microglia in tri-culture with hPSC-derived astrocytes and neurons to examine a completely human system in vitro.

FIG. 18 shows that LPS stimulation in tri-culture led to reactive cytokine release. 1 μg/mL of LPS was added to cells in tri-culture, containing microglia, astrocytes, and neurons, or to cultures containing only microglia and neurons, or only astrocytes and neurons, or only neurons. Only cultures containing microglia responded to LPS, since they were the only cell type expressing the receptor to LPS (TLR4), however in tri-culture, there was an increased release of C3 when compared to a microglia and neurons only culture. The effect may be attributed to reactive cytokines from activated microglia feeding back to astrocytes causing their reactivity and release of astrocytic C3. LPS stimulated tri and microglia/neuron only cultures also secreted other reactive cytokines, including IL-6, TNFα, GM-C SF, IL1B, and IFNγ. Cytokines were measured via ELISA.

FIG. 19 shows that tri-cultures with Alzheimer's neurons showed C3 potentiation and increased C3 release when compared to H9 control. Tri-cultures co-cultured with APP/SWe mutant neurons, a genetic model for familial Alzheimer's disease, showed increased C3 when compared to microglia and neuron only cultures, and the levels were increased when compared to cultures in which the neurons were derived from an H9 control embryonic stem cell line. In contrast, the C3 levels did not increase in the tri-culture as compared to the microglia/neuron culture in the H9 control, indicating that C3 potentiation did not occur without the disease stimulus. C3 was measured via ELISA.

FIG. 20 shows the results that GM-CSF diminished C3 release in both Alzheimer's and H9 co-cultures. GM-CSF diminished the C3 release in all cultures, both Alzheimer's and control. The cell numbers were also comparable between the GM-CSF added conditions and control conditions via immunofluorescence, indicating this effect was not due to fewer microglial cells.

FIG. 21 shows that amyloid-β burden was decreased in microglial co-cultures with selectivity for amyloid-42 peptide. Co-culture of microglial cells with Alzheimer's neurons showed decreased total amyloid beta via ELISA, particularly of the amyloid-beta 42 peptide when compared to the 40 and 38 peptides.

FIG. 22 shows that increased fluorescence of 42-488 inside microglial cells indicated increased uptake. To assay whether microglial cells phagocytose amyloid-beta, a fluorescently tagged amyloid-beta 42 peptide was used with alexa fluor 4888 and found that after 2 hours, the majority of microglial cells contained amyloid beta 42 within them via immunofluorescence.

Amyloid-beta 40, on the other hand (tagged via 555), was not found as brightly within the microglial cells, indicating that it was not phagocytosed as efficiently. This demonstrated a selectivity for amyloid beta 42 by microglial cells.

FIG. 23 shows that switching fluorophores yielded similar result: amyloid-beta 42 was taken up more by microglial cells. The fluorophores representing amyloid 42 and 40 were switched to make sure the effect of increased 42 brightness within cells was not due to a technical fluorophore brightness effect. Even with the switched fluorophores, in this experiment 42 was tagged to 555 and 40 to 488, there was more 42 brightness within microglial cells via immunofluorescence, corroborating the previous results that microglial cells phagocytosed amyloid-beta 42 selectively.

FIG. 24 provides FACS Analysis showing selectivity for amyloid-42 at baseline, and increased uptake upon GM-CSF treatment. GM-CSF treatment increased the phagocytosis of amyloid-beta 42 as well as 40 in microglial cells, and the number of cells with the amyloid-beta peptides within them were quantified using flow cytometry.

FIG. 25 shows that ALS microglia and astrocytes had increased complement C3 release at baseline. SOD1 mutant iPSC-derived ALS astrocytes and microglia were cultured alone or together, and exhibited higher levels of C3 vs. isogenic, wildtype control cell line derived astrocytes or microglia quantified via ELISA. This indicates that C3 reactivity is not unique to Alzheimer's and the presently disclosed system can be used to study other neurodegenerative diseases, where a loop between microglia, astrocytes, and neurons likely also exists.

FIGS. 26A-26D show that patterning towards primitive hematopoiesis occurred during a narrow developmental window. FIG. 26A depicts a schematic diagram of an exemplary presently disclosed method for differentiating primitive hematopoietic cells from hPSCs. FIG. 26B shows that WNT inhibition must be initiated 18 hour post WNT-activation to effectively generate KDR⁺CD235A⁺hemangioblasts. FIG. 26C shows optimized WNT activation followed by inhibition generated 30% KDR+CD235A+ population by day 3. FIG. 26D shows that only the KDR⁺CD235A⁺fraction produced CD43⁺CD235A⁺CD41⁺ EMPs by Day 6 and CD45⁺ macrophage precursors by Day 10 of differentiation.

FIGS. 27A-27F show that single cell RNA-sequencing validated the stages of microglial development within the in vitro differentiation. FIG. 27A depicts T-distributed Stochastic Neighbor Embedding (TSNE) after diffusion mapping of combined data from Day 6 and Day 10 cultures reveals distinct hemogenic endothelium (HE), erythromyeloid progenitor (EMP), erythrocyte (ERY), megakaryocyte (MK), and early macrophage (PMAC) clusters. FIG. 27B depicts Palantir analysis showing that HE clusters were the least differentiated, progressing through an EMP intermediate and branching into 3 separate differentiation trajectories towards erythrocytic (ERY), megakaryocytic (MK), and myeloid (MY) populations over pseudotime. FIG. 27C shows separate differentiation arms (ERY, MK, and MY) increasingly expressed key markers of ERY, MK, and MY identity over pseudotime. FIG. 27D shows the heatmap of gene expression data from cells along the myeloid trajectory over pseudotime compared to mouse yolk sac EMP and PMAC gene signatures (Mass et al., Science (2016); 353(6304) aaf4238) shows an enrichment for EMP genes between pseudotimes 0.16-0.77, corresponding to the in vitro human EMP clusters, and an enrichment for PMAC genes between pseudotimes 0.8-1.0, corresponding to the in vitro human PMAC clusters. FIG. 27E provides the mapping of the in vitro human data onto mouse gastrulation data shows a similarity between the human PMAC cluster and the mouse My (myeloid) cluster. FIG. 27F shows the in vitro human pMAC cluster most closely mapped with clusters present during E8.5 of mouse development.

FIGS. 28A-28J show two different methods to derive hPSC-microglia from the PMAC stage that are molecular and functionally similar to in vivo microglia. FIG. 28A depicts a schematic diagram for two methods to derive microglia from PMACS. FIG. 28B shows that the direct co-culture of Day 10 progenitors with hPSC-derived cortical neurons yielded ramified IBA1+ cells by day 4 of co-culture. FIG. 28C shows that more than 30% of cells in the co-culture expressed CX3CR1+ at Day 4 of co-culture. FIG. 28D shows that GFP+ Day 10 progenitors co-cultured with hPSC-derived cortical neurons for 6 days were over 50% CD45⁺, and more than 80% of these were CX3CR1⁺. The about 10% GFP+ population that is CD45−negative (CD45⁻) consists of about 50% CD41⁺CD235A⁺ (EMPs) and about 50% uncommitted. FIG. 28E shows maturing Day 10 progenitors in IL-34 and M-CSF without neurons yields a progressively pure population of primitive macrophages expressing CD11B (˜99%) and CX3CR1 (>85%) by 11 days in culture. PBMCs matured in parallel express CD11B (100%) but not CX3CR1⁺. FIG. 28F shows that culturing primitive macrophages with hPSC-derived cortical neurons yielded ramified IBA1+ microglial-like cells. FIG. 28G shows that co-cultured microglial cells maintained the expression of CX3CR1 and had a lower expression of CD45 than PBMC-matured macrophages co-cultured with cortical neurons, which did not express CX3CR1 and had higher CD45 expression and upregulated key microglial specific genes. FIG. 28H shows that hPSC-microglia generated from either of the methods expressed a key panel of microglial-specific genes similar to human fetal microglia (RNA from a commercial source), whereas PBMC-derived macrophages did not. The expressions of TMEM119 and SALL1 increased in the co-culture compared to microglia cultured alone. n=2. FIG. 28I shows the gene expression of bulk RNA-sequencing data of adult microglia derived from 2 different methods. n=3 per group. FIG. 28J depicts the confocal imaging (40×) of microglia co-cultured with d70 hPSC-derived cortical neurons containing inclusions of synaptic proteins (panel i); and that quantification of inclusions containing general neuronal matter tagged with RFP were greater in volume than inclusions containing synaptic protein (panel ii). n=4 per group.

FIGS. 29A-29K show that hPSC-derived microglia cultured with hPSC-derived astrocytes and neurons built a functional human tri-culture system that allowed the modeling of the neuroinflammatory axis in vivo. FIG. 29A depicts the schematic diagram of tri-culture differentiation. FIG. 29B shows that hPSC-derived astrocytes were GFAP+ and some were AQP4+. FIG. 29C shows that hPSC-derived neurons were telencephalic and maintained FOXG1. FIG. 29D shows that D50 hPSC-derived neurons expressed the cortical layer markers of TBR1 and CTIP2. FIGS. 29E represents a tri-culture showing IBA1+ microglia and GFAP+ astrocytes that interacted with MAP2+ neurons. FIG. 29F represents tri-culture having minimal cell death measured by CC3+. FIG. 29G shows the increased secretion of C3 protein in the tri-culture (TRI), which was measured by ELISA compared to microglia/neuron (M/N) cultures which is exacerbated upon LPS treatment. C3 was not secreted in astrocyte/neuron (A/N) and neuron only (N) cultures. n=4 per group, ANOVA, Sidak's post-hoc test. FIG. 29H shows that LPS stimulation induced the secretion of other inflammatory cytokines. FIG. 29I shows that C3 KO microglia did not secrete C3 protein by ELISA. FIG. 29J shows that C3KOA cultures had decreased C3 release compared to wildtype (wt) tri-cultures but secreted more C3 than M/N cultures. C3KOM cultures had the low levels of minimal C3 release. C3 release in C3KOA cultures was reduced by LPS treatment compared to tri-cultures; C3KOM had low C3 release compared to the other groups. FIG. 29K depicts the neuroinflammatory loop in tri-culture, which was initiated by microglia signaling to astrocytes that signaled back to microglia leading to the increase in C3 release.

FIGS. 30A-30H depict that the tri-culture model of Alzheimer's Disease (AD) showed the increase in C3 release in AD tri-cultures caused by microglial signaling to astrocytes. FIG. 30A shows that isogenic APPSWE+/+neurons expressed the telencephalic marker FOXG1. FIG. 30B shows the cortical layer markers of CTIP2 and TBR1. FIG. 30C shows the total amyloid amount secreted by D50 isogenic APPSWE+/+neurons and WT controls. n=2, Student's t-test. FIG. 30D shows that isogenic APPSWE+/+tri-cultures with d80 neurons (MAP2+), wildtype hPSC-derived microglia (IBA1+) and astrocytes (GFAP+). FIG. 30E shows that APPSWE+/+tri-cultures secreted more C3 than isogenic control tri-cultures. n=3, ANOVA with post-hoc Sidak's. FIG. 30F provides that C3KOA tri-cultures with APPSWE+/+neurons showed lower C3 secretion compared to APPSWE+/+tri-cultures with wildtype astrocytes. C3KOM APPSWE+/+tri-cultures secreted low levels of C3. n=3 per group, ANOVA with post-hoc Sidak's. FIG. 30G shows APPSWE+/+cultures containing microglia expressed the higher levels of C1QA deposition compared to isogenic control cultures. FIG. 30H depicts a neuroinflammatory loop in the in vitro model of AD, where APPSWE+/+neurons activated microglia which activated astrocytes leading to increased C3 release.

FIG. 31A-31C show the generation of hematopoietic identity cells. FIG. 31A shows that the addition of erythropoietin (EPO) from Day 6 allowed the emergence of CD235A+ erythrocytes at Day 10 of differentiation. FIG. 31B shows that round hematopoietic cells progressively proliferate in semi-suspension by Day 10. FIG. 31C shows that VE-cadherin+hemogenic endothelium was present in differentiating cultures.

FIGS. 32A-32B show the generation of GFP⁺ hematopoietic cells from a GPI⁻H2B⁻GFP hPSC line. FIG. 32A shows that targeted lines containing GFP after H2B produced a 1 kb product upon PCR of the locus. FIG. 32B shows that GPI-H2B-GFP hPSC line at the pluripotent stage expressed GFP in every cell.

FIG. 33A-33C show representative brightfield and immunofluorescent images of microglial cells. FIG. 33A shows all differentiating cells by 11 days of culture in IL-34 and M-CSF under serum or serum-free conditions were adherent and the cells displayed an elongated morphology. FIG. 33B shows that all cells expressed the myeloid transcription factor PU.1. FIG. 33C shows that microglial cells upregulated IBA1 upon co-cultured and developed a ramified morphology.

FIGS. 34A-34B show the results of single-cell RNA sequencing of the co-cultured hPSC-derived microglial cells. FIG. 34A shows that pairwise distances between cells in the microglial sample fell in a homogenous unimodal distribution. FIG. 34B shows pairwise distances between cells in the heterogenous Day 10 sample had multiple peaks.

FIGS. 35A-35B show phagocytosis of cells when challenged with yeast-antigen zymosan. FIG. 35A shows microglial cells displaying zymosan-conjugated fluorescent beads as inclusions within 4 hours. FIG. 35B shows that astrocyte control did not display fluorescent bead inclusions.

FIGS. 36A-36B show the pre-synaptic and post-synaptic gene expression of hPSC-derived cortical neurons. FIG. 36A shows hPSC-derived cortical neurons at D70 showing a punctate distribution of the pre-synaptic SYNI and post-synaptic HOMER1, and putative synapses where both are side-by-side (white arrow). FIG. 36B shows that D70 hPSC-derived cortical neurons expressed the post-synaptic marker PSD95 in a punctate distribution.

FIGS. 37A-37C show C3 secretion in tri-cultures under different conditions. FIG. 37A shows tri-cultures with increased numbers of astrocytes plated (50K) contained fewer microglial cells (IBA1+) at Day 7 (5×). FIG. 37B shows that the secretion of C3 in the tri-culture NB/BAGC and NB:N2 base media formulations were the lowest among all tested cell cultures. FIG. 37C shows that hPSC-derived neurons killed by 70% methanol incubation for 30 minutes showed bright CC3+, live cells did not.

FIG. 38A-38C show the quantification of IBA+ cells by immunofluorescence using a high-content imaging microscope. FIG. 38A shows that the cell scoring of % IBA1+/DAPI in microglia/neuron (M/N) cultures of M/N was assessed higher compared to tri-cultures at Day 7 using ImageExpress microscopy of 9 fields of a well shows. FIG. 38B shows the representative image of quantification of 9 fields of a well for cell scoring showing DAPI and IBA1 staining. FIG. 38C shows a cell scoring illustrating similar numbers of IBA1+ microglia and GFAP+ astrocytes between the different tri-cultures (TM, C3KOM, C3KOA).

### 4. SUMMARY

The present disclosure relates to methods for generating microglial cells derived from stem cells (e.g., human stem cells), microglial cells obtained from such methods and compositions comprising thereof, uses of such microglial cells for disease modeling and for treating microglia related disorders.

The present disclosure provides in vitro methods for inducing differentiation of stem cells. In certain embodiments, the method comprises a) contacting stem cells with at least one activator of Wingless (Wnt) signaling for up to about 24 hours; b) contacting the cells with at least one inhibitor of Wnt signaling and at least one hematopoiesis-promoting cytokine to obtain a population of differentiated cells, wherein the differentiated cells are selected from the group consisting of cells expressing at least one erythromyeloid progenitor (EMP) marker, cells expressing at least one pre-macrophage marker, and a combination thereof; and c) inducing differentiation of the differentiated cells to cells expressing at least one microglial marker.

In certain embodiments, c) inducing differentiation of the differentiated cells to cells expressing at least one microglial marker comprises culturing the differentiated cells with neurons for at least about 5 days. In certain embodiments, c) inducing differentiation of the differentiated cells to cells expressing at least one microglial marker comprises culturing the differentiated cells with neurons for 4 days. In certain embodiments, c) inducing differentiation of the differentiated cells to cells expressing at least one microglial marker comprises contacting the differentiated cells with at least one macrophage-promoting cytokine for at least about 5 days; and culturing the cells with neurons for at least about 5 days. In certain embodiments, the method comprises culturing the cells with neurons for at least 4 days.

In certain embodiments, the cells are contacted with the at least one activator of Wnt signaling for about 20 hours. In certain embodiments, the cells are contacted with the at least one activator of Wnt signaling for 18 hours.

In certain embodiments, the cells are contacted with the at least one inhibitor of Wnt signaling for at least about 1 day and up to about 5 days. In certain embodiments, the cells are contacted with the at least one inhibitor of Wnt signaling for at least 1 day and up to 4 days. In certain embodiments, the cells are contacted with the at least one inhibitor of Wnt signaling for at least about 2 days.

In certain embodiments, the cells are contacted with the at least one hematopoiesis-promoting cytokine for at least about 1 day and up to about 10 days. In certain embodiments, the cells are contacted with the at least one hematopoiesis-promoting cytokine for at least 3 days and up to 11 days. In certain embodiments, the cells are contacted with the at least one macrophage-promoting cytokine for 7 days, 8 days, 9 days, 10 days, or 11 days. In certain embodiments, the cells are cultured with the neurons for about 5 days. In certain embodiments, the cells are cultured with the neurons for 4 days or 5 days.

In certain embodiments, contacting the stem cells with the at least one activator of Wnt signaling generates cells expressing at least one mesoderm progenitor marker. In certain embodiments, said at least one mesoderm progenitor marker is selected from the group consisting of comprises Brachyury, KDR and combinations thereof In certain embodiments, contacting the cells with the at least one inhibitor of Wnt signaling generates cells expressing at least one primitive hematopoietic precursor marker. In certain embodiments, said at least one primitive hematopoietic precursor marker is selected from the group consisting of KDR, CD235A, and combinations thereof. In certain embodiments, contacting the cells with the at least one hematopoiesis-promoting cytokine further generates cells expressing at least one erythromyeloid progenitor (EMP) marker. In certain embodiments, the cells are contacted with the at least one hematopoiesis-promoting cytokine for at least about 1 day and up to about 5 days and/or up to about 10 days to generate the cells expressing at least one EMP marker. In certain embodiments, the at least one EMP marker is selected from the group consisting of Kit, CD41, CD235A, CD43, and combinations thereof. In certain embodiments, the cells expressing at least one EMP marker do not express CD45. In certain embodiments, the at least one pre-macrophage marker is selected from the group consisting of CD45, CSF1R, and combinations thereof. In certain embodiments, the cells expressing at least one pre-macrophage marker do not express Kit. In certain embodiments, the at least one microglial marker is selected from the group consisting of CX3CR1, PU.1, CD45, IBA1, P2RY12, TMEM119, SALL1, GPR34, C1QA, CD68, CD45, and combinations thereof. In certain embodiments, the at least one macrophage marker is selected from the group consisting of CD11B, DECTIN, CD14, PU.1, CX3CR1, CD45 and combinations thereof.

In certain embodiments, the at least one activator of Wnt signaling lowers glycogen synthase kinase 3β (GSK3β) for activation of Wnt signaling. In certain embodiments, the at least one activator of Wnt signaling is selected from the group consisting of CHIR99021, Wnt-1, WNT3A, Wnt4, Wnt5a, WAY-316606, IQ1, QS11, SB-216763, BIO(6-bromoindirubin-3′-oxime), LY2090314, DCA, 2-amino-4-[3,4-(methylenedioxy)benzyl-amino]-6-(3-methoxyphenyl)pyrimidine, (hetero)arylpyrimidines, derivatives thereof, and combinations thereof. In certain embodiments, the at least one activator of Wnt signaling is CHIR99021.

In certain embodiments, the at least one inhibitor of Wnt signaling is selected from the group consisting of XAV939, IWP2, DKK1, IWR1, peptide (Nile et al peptide (Nile et al Nat Chem Biol. 2018 June; 14(6):582-590), porccupine inhibitors, LGK974, C59, ETC-159, Ant1.4Br/Ant 1.4Cl, niclosamide, apicularen, bafilomycin, G007-LK, G244-LM, pyrvinium, NSC668036, 2,4-diamino-quinazoline, Quercetin, ICG-001, PKF115-584, BC2059, Shizokaol D derivatives thereof, and combinations thereof. In certain embodiments, the at least one inhibitor of Wnt signaling is IWP2.

In certain embodiments, the at least one hematopoiesis-promoting cytokine is selected from the group consisting of VEGF, FGF, SCF, interleukins, TPO, and combinations thereof In certain embodiments, the interleukins are selected from the group consisting of IL-1, IL-2, IL-3, IL-4, IL-5, IL-6, IL-7, IL-8, IL-9, IL-10, IL-11, IL-12, IL-13, IL-15, and combinations thereof. In certain embodiments, the interleukins are selected from the group consisting of IL-6, IL-3, and combinations thereof. In certain embodiments, the FGF is selected from the group consisting of FGF1, FGF2, FGF3, FGF4, FGF7, FGF8, FGF10, FGF18, and combinations thereof. In certain embodiments, the FGF is FGF2.

In certain embodiments, the at least one macrophage-promoting cytokine is selected from the group consisting of M-CSF, IL-34 and combinations thereof.

In certain embodiments, the cells are contacted with the at least one activator of Wnt signaling at a concentration between about 1 μM and about 6 μM. In certain embodiments, the cells are contacted with the at least one inhibitor of Wnt signaling at a concentration between about 1 μM and about 10 μM. In certain embodiments, the cells are contacted with the at least one hematopoiesis-promoting cytokine at a concentration between about 1 ng/ml and about 50 ng/ml. In certain embodiments, the cells are contacted with the at least one hematopoiesis-promoting cytokine at a concentration between about 1 ng/ml and about 400 ng/ml. In certain embodiments, the cells are contacted with the at least one macrophage-promoting cytokine at a concentration between about 1 ng/ml and about 200 ng/ml.

In another aspect, the present disclosure provides a cell population of in vitro differentiated cells expressing at least one microglial marker, wherein said in vitro differentiated cells are derived from stem cells according to the differentiation method presently disclosed herein. Also provided are compositions comprising such cell population.

In another aspect, the present disclosure provides a kit for inducing differentiation of stem cells, comprising: (a) at least one inhibitor of Wnt signaling; (b) at least one activator of Wnt signaling; (c) at least one hematopoiesis-promoting cytokine; and (d) neurons. In certain embodiments, the kit further comprises (e) at least one macrophage-promoting cytokine.

In certain embodiments, the kit further comprises instructions for inducing differentiation of stem cells into cells expressing at least one microglial marker.

In another aspect, the present disclosure provides a composition comprising a population of in vitro differentiated cells, wherein at least about 50% of the cells comprised in the population express at least one microglial marker, and wherein less than about 25% of the cells comprised in the population express at least one marker selected from the group consisting of stem cells markers, mesoderm progenitor markers, primitive hematopoietic precursor markers, EMP markers, pre-macrophage markers, macrophage markers.

In another aspect, the present disclosure provides methods of preventing and/or treating a neurodegenerative disease in a subject. In certain embodiments, the method comprises administering to a subject one of the followings: (a) the population of differentiated microglial cell disclosed herein; (b) a composition disclosed herein; and (c) the composition of disclosed herein. In certain embodiments, the method comprises administering to a subject a colony-stimulating factor (CSF).

In certain embodiments, the neurodegenerative disease is Alzheimer's disease or Amyotrophic Lateral Sclerosis (ALS). In certain embodiments, the neurodegenerative disease is an Alzheimer's disease.

In certain embodiments, the CSF is selected from the group consisting of colony-stimulating factor (GM-CSF), and M-CSF. In certain embodiments, the CSF is a GM-CSF.

In another aspect, the present disclosure provides a CSF for use in preventing and/or treating a neurodegenerative disease.

In another aspect, the present disclosure provides use of a CSF for the manufacture of a medicament for preventing and/or treating a neurodegenerative disease.

In another aspect, the present disclosure provides a method for screening a therapeutic compound for treating a neurodegenerative disease comprising: (a) contacting a population of differentiated microglial cell of claim 34 with a test compound, wherein the microglial cells are derived from stem cells obtained from a subject with the neurodegenerative disease; and (b) measuring functional activity of the microglial cells, wherein a change in the functional activity of the microglial cells indicates that the test compound is likely to be capable of treating a neurodegenerative disease.

In certain embodiments, the change is a decrease or an increase. In certain embodiments, functional activity of the microglial cells comprises release of complement C3. In certain embodiments, a decrease in the complement C3 released from the microglial cells indicates that the therapeutic compound is likely to be capable of treating a neurodegenerative disease. In certain embodiments, the functional activity of the microglial cells comprises amyloid-beta phagocytosis by the microglial cells. In certain embodiments, the neurodegenerative disease is an Alzheimer's disease.

In another aspect, the present disclosure provides a method for screening a therapeutic compound for treating a neurodegenerative disease comprising: (a) contacting a test compound with a composition comprising the differentiated microglial cell of disclosed herein, a population of astrocytes, and a population of neurons; and (b) measuring neurotoxicity of the neurons, wherein a decrease in the neurotoxicity of the neurons after the contact with the test compound indicates that the test compound is likely to be capable of treating a neurodegenerative disease. In certain embodiments, the neurodegenerative disease is an ALS disease. In certain embodiments, the microglial cell induces reactive astrocytes, which induces neurotoxicity to neurons.

### 5. DETAILED DESCRIPTION

The present disclosure provides a step-wise developmental paradigm that recapitulates yolk sac primitive hematopoiesis, isolates pre-macrophages (pMacs) before maturation into macrophages, and cultures these cells in an in vitro neural niche to generate bona fide human microglial cells in as little as 16 days.

For purposes of clarity of disclosure and not by way of limitation, the detailed description is divided into the following subsections:

5.1 Definitions;

5.2 Methods of Differentiating Stem Cells;

5.3 Compositions Comprising Microglia;

5.4 Methods of Treatment; and

5.5 Kits

5.6 Methods of Screening Therapeutic Compounds

### 5.1 Definitions.

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold, or within 2-fold, of a value.

As used herein, the term “signaling” in reference to a “signal transduction protein” refers to a protein that is activated or otherwise affected by ligand binding to a membrane receptor protein or some other stimulus. Examples of signal transduction protein include, but are not limited to, a Fibroblast Growth Factor (FGF), a SMAD, a Wingless (Wnt) complex protein, including beta-catenin, NOTCH, transforming growth factor beta (TGFβ), Activin, Nodal and glycogen synthase kinase 3β (GSK3β) proteins. For many cell surface receptors or internal receptor proteins, ligand-receptor interactions are not directly linked to the cell's response. The ligand activated receptor can first interact with other proteins inside the cell before the ultimate physiological effect of the ligand on the cell's behavior is produced. Often, the behavior of a chain of several interacting cell proteins is altered following receptor activation or inhibition. The entire set of cell changes induced by receptor activation is called a signal transduction mechanism or signaling pathway.

As used herein, the term “signals” refer to internal and external factors that control changes in cell structure and function. They can be chemical or physical in nature.

As used herein, the term “ligands” refers to molecules and proteins that bind to receptors, e.g., TFGβ, Activin, Nodal, bone morphogenic proteins (BMPs), etc.

“Inhibitor” as used herein, refers to a compound or molecule (e.g., small molecule, peptide, peptidomimetic, natural compound, siRNA, anti-sense nucleic acid, aptamer, or antibody) that interferes with (e.g., reduces, decreases, suppresses, eliminates, or blocks) the signaling function of the molecule or pathway. An inhibitor can be any compound or molecule that changes any activity of a named protein (signaling molecule, any molecule involved with the named signaling molecule, a named associated molecule, such as a Wingless (Wnt)) (e.g., including, but not limited to, the signaling molecules described herein), for one example, via directly contacting Wnt signaling, contacting Wnt mRNA, causing conformational changes of Wnt, decreasing Wnt protein levels, or interfering with Wnt interactions with signaling partners (e.g., including those described herein), and affecting the expression of Wnt target genes (e.g. those described herein). Inhibitors also include molecules that indirectly regulate Wnt biological activity by intercepting upstream signaling molecules (e.g., within the extracellular domain, examples of a signaling molecule. Inhibitors are described in terms of competitive inhibition (binds to the active site in a manner as to exclude or reduce the binding of another known binding compound) and allostenc inhibition (binds to a protein in a manner to change the protein conformation in a manner which interferes with binding of a compound to that protein's active site) in addition to inhibition induced by binding to and affecting a molecule upstream from the named signaling molecule that in turn causes inhibition of the named molecule. An inhibitor can be a “direct inhibitor” that inhibits a signaling target or a signaling target pathway by actually contacting the signaling target.

“Activators”, as used herein, refer to compounds that increase, induce, stimulate, activate, facilitate, or enhance activation the signaling function of the molecule or pathway, e.g., Wnt signaling, or FGF signaling.

As used herein, the term “derivative” refers to a chemical compound with a similar core structure.

As used herein, the term “a population of cells” or “a cell population” refers to a group of at least two cells. In non-limiting examples, a cell population can include at least about 10, at least about 100, at least about 200, at least about 300, at least about 400, at least about 500, at least about 600, at least about 700, at least about 800, at least about 900, at least about 1000 cells. The population may be a pure population comprising one cell type. Alternatively, the population may comprise more than one cell type, for example a mixed cell population.

As used herein, the term “stem cell” refers to a cell with the ability to divide for indefinite periods in culture and to give rise to specialized cells. A human stem cell refers to a stem cell that is from a human.

As used herein, the term “embryonic stem cell” refers to a primitive (undifferentiated) cell that is derived from preimplantation-stage embryo, capable of dividing without differentiating for a prolonged period in culture and are known to develop into cells and tissues of the three primary germ layers. A human embryonic stem cell refers to an embryonic stem cell that is from a human. As used herein, the term “human embryonic stem cell” or “hESC” refers to a type of pluripotent stem cells (“PSCs”) derived from early stage human embryos, up to and including the blastocyst stage, that is capable of dividing without differentiating for a prolonged period in culture and are known to develop into cells and tissues of the three primary germ layers.

As used herein, the term “embryonic stem cell line” refers to a population of embryonic stem cells which have been cultured under in vitro conditions that allow proliferation without differentiation for up to days, months to years.

As used herein, the term “totipotent” refers to an ability to give rise to all the cell types of the body plus all of the cell types that make up the extraembryonic tissues such as the placenta.

As used herein, the term “multipotent” refers to an ability to develop into more than one cell type of the body.

As used herein, the term “pluripotent” refers to an ability to develop into the three developmental germ layers of the organism including endoderm, mesoderm, and ectoderm.

As used herein, the term “induced pluripotent stem cell” or “iPSC” refers to a type of pluripotent stem cell, similar to an embryonic stem cell, formed by the introduction of certain embryonic genes (such as a OCT4, SOX2, and KLF4 transgenes) (see, for example, Takahashi and Yamanaka Cell 126, 663-676 (2006), herein incorporated by reference) into a somatic cell, for examples, CI 4, C72, and the like.

As used herein, the term “somatic cell” refers to any cell in the body other than gametes (egg or sperm); sometimes referred to as “adult” cells. As used herein, the term “somatic (adult) stem cell” refers to a relatively rare undifferentiated cell found in many organs and differentiated tissues with a limited capacity for both self-renewal (in the laboratory) and differentiation. Such cells vary in their differentiation capacity, but it is usually limited to cell types in the organ of origin.

As used herein, the term “neuron” refers to a nerve cell, the principal functional units of the nervous system. A neuron consists of a cell body and its processes—an axon and at least one dendrites. Neurons transmit information to other neurons or cells by releasing neurotransmitters at synapses.

As used herein, the term “proliferation” refers to an increase in cell number. As used herein, the term “undifferentiated” refers to a cell that has not yet developed into a specialized cell type. As used herein, the term “differentiation” refers to a process whereby an unspecialized embryonic cell acquires the features of a specialized cell such as a heart, liver, or muscle cell. Differentiation is controlled by the interaction of a cell's genes with the physical and chemical conditions outside the cell, usually through signaling pathways involving proteins embedded in the cell surface.

As used herein, the term “directed differentiation” refers to a manipulation of stem cell culture conditions to induce differentiation into a particular (for example, desired) cell type, such as EMPs.

As used herein, the term “directed differentiation” in reference to a stem cell refers to the use of small molecules, growth factor proteins, and other growth conditions to promote the transition of a stem cell from the pluripotent state into a more mature or specialized cell fate (e.g. pMacs, macrophages, microglia, etc.).

As used herein, the term “inducing differentiation” in reference to a cell refers to changing the default cell type (genotype and/or phenotype) to a non-default cell type (genotype and/or phenotype). Thus, “inducing differentiation in a stem cell” refers to inducing the stem cell (e.g., human stem cell) to divide into progeny cells with characteristics that are different from the stem cell, such as genotype (e.g., change in gene expression as determined by genetic analysis such as a microarray) and/or phenotype (e.g., change in expression of a protein, such as microglial marker(s)).

As used herein, the term “cell culture” refers to a growth of cells in vitro in an artificial medium for research or medical treatment.

As used herein, the term “culture medium” refers to a liquid that covers cells in a culture vessel, such as a Petri plate, a multi-well plate, and the like, and contains nutrients to nourish and support the cells. Culture medium may also include growth factors added to produce desired changes in the cells.

As used herein, the term “contacting” cells with a compound (e.g., at least one inhibitor, activator, and/or inducer) refers to placing the compound in a location that will allow it to touch the cell. The contacting may be accomplished using any suitable methods. For example, contacting can be accomplished by adding the compound to a tube of cells. Contacting may also be accomplished by adding the compound to a culture medium comprising the cells. Each of the compounds (e.g., the inhibitors, activators, and inducers disclosed herein) can be added to a culture medium comprising the cells as a solution (e.g., a concentrated solution). Alternatively, or additionally, the compounds (e.g., the inhibitors, activators, and inducers disclosed herein) as well as the cells can be present in a formulated cell culture medium.

As used herein, the term “in vitro” refers to an artificial environment and to processes or reactions that occur within an artificial environment. In vitro environments exemplified, but are not limited to, test tubes and cell cultures. As used herein, the term “in vivo” refers to the natural environment (e.g., an animal or a cell) and to processes or reactions that occur within a natural environment, such as embryonic development, cell differentiation, neural tube formation, etc.

As used herein, the term “expressing” in relation to a gene or protein refers to making an mRNA or protein which can be observed using assays such as microarray assays, antibody staining assays, and the like.

As used herein, the term “marker” or “cell marker” refers to gene or protein that identifies a particular cell or cell type. A marker for a cell may not be limited to one marker, markers may refer to a “pattern” of markers such that a designated group of markers may identity a cell or cell type from another cell or cell type.

As used herein, the term “derived from” or “established from” or “differentiated from” when made in reference to any cell disclosed herein refers to a cell that was obtained from (e.g., isolated, purified, etc.) a parent cell in a cell line, tissue (such as a dissociated embryo, or fluids using any manipulation, such as, without limitation, single cell isolation, cultured in vitro, treatment and/or mutagenesis using for example proteins, chemicals, radiation, infection with virus, transfection with DNA sequences, such as with a morphogen, etc., selection (such as by serial culture) of any cell that is contained in cultured parent cells. A derived cell can be selected from a mixed population by virtue of response to a growth factor, cytokine, selected progression of cytokine treatments, adhesiveness, lack of adhesiveness, sorting procedure, and the like.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “disease” refers to any condition or disorder that damages or interferes with the normal function of a cell, tissue, or organ.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder

### 5.2. Method of Differentiating Stem Cells

The present disclosure provides for in vitro methods for inducing differentiation of stem cells (e.g., human stem cells). Non-limiting examples of human stem cells include human embryonic stem cells (hESC), human pluripotent stem cell (hPSC), human induced pluripotent stem cells (hiPSC), human parthenogenetic stem cells, primordial germ cell-like pluripotent stem cells, epiblast stem cells, F-class pluripotent stem cells, somatic stem cells, cancer stem cells, or any other cell capable of lineage specific differentiation. In certain embodiments, the human stem cell is a human embryonic stem cell (hESC). In certain embodiments, the human stem cell is a human induced pluripotent stem cell (hiPSC).

The present disclosure is directed to stem cell-derived microglial cells. In certain embodiments, the differentiation of stem cells to microglial cells include in vitro differentiation of stem cells to cells expressing at least one mesoderm progenitor marker, in vitro differentiation to cells expressing at least one primitive hematopoietic precursor marker, in vitro differentiation cells expressing at least one erythromyeloid progenitor (EMP) marker, in vitro differentiation to cells expressing at least one pre-macrophage (also known as macrophage precursor) (pMac) marker, and in vitro differentiation of cells expressing at least one pMac marker to cells expressing at least one microglial marker. In certain embodiments, the differentiation of stem cells to microglial cells further includes in vitro differentiation of cells expressing at least one pMac marker to cells expressing at least one macrophage marker, and in vitro differentiation of cells expressing at least one macrophage marker to cells expressing at least one microglial marker.

In certain embodiments, the present disclosure provides methods for inducing differentiation of stem cells, comprising a) contacting stem cells with at least one activator of Wingless (Wnt) signaling; b) contacting the cells with at least one inhibitor of Wnt signaling and at least one hematopoiesis-promoting cytokine to obtain a population of differentiated cells selected from the group consisting of cells expressing at least one erythromyeloid progenitor (EMP) marker, cells expressing at least one pre-macrophage marker, and a combination thereof; and c) inducing the differentiation of the differentiated cells to cells expressing at least one microglial marker. In certain embodiments, inducing the differentiation of the differentiated cells to cells expressing at least one microglial marker comprises culturing the differentiated cells with neurons. In certain embodiments, inducing the differentiation of the differentiated cells to cells expressing at least one microglial marker comprises contacting the differentiated cells with at least one macrophage-promoting cytokine; and culturing the cells with neurons.

### 5.2.1. Differentiation of Stem Cells to Mesoderm Progenitors

In certain embodiments, the cells expressing at least one mesoderm progenitor marker are in vitro differentiated from stems cells by contacting stem cells (e.g., human stem cells) with at least one activator of Wnt signaling (e.g., CHIR99021) (referred to as “Wnt activator”). In certain embodiments, the stem cells are further contacted with at least one BMP active agent, e.g., a BMP molecule (e.g., BMP4), and at least one activin protein (e.g., Activin A).

Non-limiting examples of mesoderm progenitor markers include Brachyury, KDR and combinations thereof.

As used herein, the term “Wnt” or “wingless” in reference to a ligand refers to a group of secreted proteins (i.e. Intl (integration 1) in humans) capable of interacting with a Wnt receptor, such as a receptor in the Frizzled and LRPDerailed/RYK receptor family. As used herein, the term “Wnt” or “wingless” in reference to a signaling pathway refers to a signal pathway composed of Wnt family ligands and Wnt family receptors, such as Frizzled and LRPDerailed/RYK receptors, mediated with or without β-catenin. For the purposes described herein, a preferred Wnt signaling pathway includes mediation by β-catenin, e.g., WNT/-catenin.

In certain embodiments, the at least one Wnt activator lowers glycogen synthase kinase 3β (GSK3β) for activation of Wnt signaling. Thus, the Wnt activator can be a GSK3β inhibitor. A GSK3P inhibitor is capable of activating a WNT signaling pathway, see e.g., Cadigan, et al., J Cell Sci. 2006;119:395-402; Kikuchi, et al., Cell Signaling. 2007;19:659-671, which are incorporated by reference herein in their entireties. As used herein, the term “glycogen synthase kinase 3β inhibitor” refers to a compound that inhibits a glycogen synthase kinase 3β enzyme, for example, see, Doble, et al., J Cell Sci. 2003;116:1175-1186, which is incorporated by reference herein in its entirety.

Non-limiting examples of Wnt activators or GSK3β inhibitors are disclosed in WO2011/149762, Chambers (2012), and Calder et al., J Neurosci. 2015 Aug. 19;35(33):11462-81, which are incorporated by reference in their entireties. In certain embodiments, the at least one Wnt activator is a small molecule selected from the group consisting of CHIR99021, derivatives thereof, and mixtures thereof. In certain embodiments, the at least one Wnt activator is CHIR99021.

“CHIR99021” (also known as “aminopyrimidine” or “3-[3-(2-Carboxyethyl)-4-methylpyrrol-2-methylidenyl]-2-indolinone”) refers to IUPAC name 6-(2-(4-(2,4-dichlorophenyl)-5-(4-methyl-1H-imidazol-2-yl)pyrimidin-2-ylamino) ethylamino)nicotinonitrile with the following formula.

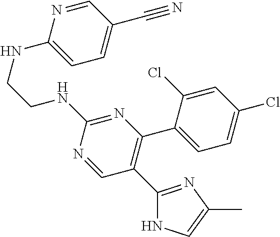 [SMILES: Cc1c[nH]c(-c2cnc(NCCNc3ccc(C#N)cn3)nc2-c2ccc(Cl)cc2Cl)n1]

CHIR99021 is highly selective, showing nearly thousand-fold selectivity against a panel of related and unrelated kinases, with an IC50=6.7 nM against human GSK3β and nanomolar IC50 values against rodent GSK3β homologs.

Non-limiting examples of Wnt activators include CHIR99021, Wnt-1, WNT3A, Wnt4, Wnt5a, WAY-316606, IQ1, QS11, SB-216763, BIO(6-bromoindirubin-3′-oxime), LY2090314, DCA, 2-amino-4-[3,4-(methylenedioxy)benzyl-amino]-6-(3-methoxyphenyl)pyrimidine, (hetero)arylpyrimidines, derivatives thereof, and combinations thereof. In certain embodiments, the Wnt activator is CHIR99021.

In certain embodiments, the BMP active agent is a BMP molecule. Non-limiting examples of BMPs include BMP1, BMP2, BMP3, BMP4, BMP5, BMP6, BMP7, BMP8a, BMP8b, BMP10, BMP11, BMP15, derivatives thereof, and mixtures thereof In certain embodiments, the BMP active agent is BMP4.

None-limiting examples of activin proteins include Activin A, Activin AB, Activin C, Activin B, and Activin AC, derivatives thereof, and combinations thereof. In certain embodiments, the activin protein is Activin A.

For in vitro differentiation of stem cells to cells expressing at least one mesoderm progenitor marker, the stem cells can be contacted with the at least one activator of Wnt signaling for up to about 10 hours, up to about 15 hours, up to about 20 hours, or up to about 25 hours. In certain embodiments, the stem cells are contacted with the at least one activator of Wnt signaling for up to about 20 hours. In certain embodiments, the stem cells are contacted with the at least one activator of Wnt signaling for at least about 10 hours, at least about 15 hours, at least about 20 hours, or at least about 25 hours. In certain embodiments, the stem cells are contacted with the at least one activator of Wnt signaling for between about 10 hours and about 25 hours, between about 10 hours and about 15 hours, between about 10 hours and about 20 hours, between about 15 hours and about 25 hours, or between about 15 hours and about 20 hours. In certain embodiments, the stem cells are contacted with the at least one activator of Wnt signaling for between about 10 hours and about 20 hours. In certain embodiments, the stem cells are contacted with the at least one activator of Wnt signaling for between about 15 hours and about 20 hours. In certain embodiments, the stem cells are contacted with the at least one activator of Wnt signaling for about 10 hours, about 15 hours or about 20 hours, or about 25 hours. In certain embodiments, the stem cells are contacted with the at least one activator of Wnt signaling for about 15 hours or about 20 hours. In certain embodiments, the stem cells are contacted with the at least one activator of Wnt signaling for 16 hours, 17 hours, 18 hours, 19 hours, or 20 hours. In certain embodiments, the stem cells are contacted with the at least one activator of Wnt signaling for 18 hours.

In certain embodiments, for in vitro differentiation of stem cells to cells expressing at least one mesoderm progenitor marker, the stem cells can be further contacted with at least one BMP active agent (e.g., BMP4). In certain embodiments, the stem cells are further contacted with at least one activin protein (e.g., Activin A). In certain embodiments, the stem cells are contacted with the Wnt activator(s), BMP active agent(s) and Activin protein(s) concurrently or simultaneously. In certain embodiments, the stem cells are contacted with the at least one Wnt activator, the at least one BMP active agent, and the at least one activin protein concurrently for up to about 10 hours, up to about 15 hours, up to about 20 hours, or up to about 25 hours. In certain embodiments, the stem cells are contacted with the at least one activator of Wnt signaling, at least one BMP active agent (e.g., BMP4), and at least one activin protein (e.g., Activin A) concurrently for at least about 10 hours, at least about 15 hours, at least about 20 hours, or at least about 25 hours. In certain embodiments, the stem cells are contacted with the at least one activator of Wnt signaling, at least one BMP active agent (e.g., BMP4), and at least one activin protein (e.g., Activin A) concurrently for between about 10 hours and about 25 hours, between about 10 hours and about 15 hours, between about 10 hours and about 20 hours, between about 15 hours and about 25 hours, or between about 15 hours and about 20 hours. In certain embodiments, the stem cells are contacted with the at least one activator of Wnt signaling, at least one BMP active agent (e.g., BMP4), and at least one activin protein (e.g., Activin A) concurrently for between about 15 hours and about 20 hours. In certain embodiments, the stem cells are contacted with the at least one Wnt activator, the at least one BMP active agent, and the at least one activin protein concurrently for about 15 hours or about 20 hours. In certain embodiments, the stem cells are contacted with the at least one Wnt activator, the at least one BMP active agent, and the at least one activin protein concurrently for 10 hours, 11 hours, 12 hours, 13 hours, 14 hours, 15 hours,16 hours, 17 hours, 18 hours, 19 hours, 20 hours, 21 hours, 22 hours, 23 hours, 24 hours, or 25 hours. In certain embodiments, the stem cells are contacted with the at least one activator of Wnt signaling, at least one BMP active agent (e.g., BMP4), and at least one activin protein (e.g., Activin A) concurrently for about 20 hours. In certain embodiments, the stem cells are contacted with the at least one activator of Wnt signaling, at least one BMP active agent (e.g., BMP4), and at least one activin protein (e.g., Activin A) concurrently for 18 hours.

In certain embodiments, the stem cells are contacted with the at least one activator of Wnt signaling in a concentration of from about 1 μM to about 100 from about 1 μM to about 20 from about 1 μM to about 15 from about 1 μM to about 10 from about 1 μM to about 6 from about 6 μM to about 10 from about 6 μM to about 15 from about 15 μM to about 20 from about 20 μM to about 30 from about 30 μM to about 40 from about 40 μM to about 50 from about 50 μM to about 60 from about 60 μM to about 70 from about 70 μM to about 80 from about 80 μM to about 90 or from about 90 μM to about 100 μM. In certain embodiments, the stem cells are contacted with the at least one activator of Wnt signaling in a concentration of from about 1 μM to about 6 μM. In certain embodiments, the stem cells are contacted with the at least one activator of Wnt signaling in a concentration of about 3 μM.

In certain embodiments, the stem cells are contacted with the at least one BMP active agent in a concentration of from about 1 ng/ml to about 100 ng/ml, from about 1 ng/ml to about 20 ng/ml, from about 1 ng/ml to about 15 ng/ml, from about 1 ng/ml to about 10 ng/ml, from about 1 ng/ml to about 5 ng/ml, from about 5 ng/ml to about 10 ng/ml, from about 5 ng/ml to about 15 ng/ml, from about 15 ng/ml to about 25 ng/ml, from about 15 ng/ml to about 20 ng/ml, from about 20 ng/ml to about 30 ng/ml, from about 30 ng/ml to about 40 ng/ml, from about 40 ng/ml to about 50 ng/ml, from about 50 ng/ml to about 60 ng/ml, from about 60 ng/ml to about 70 ng/ml, from about 70 ng/ml to about 80 ng/ml, from about 80 ng/ml to about 90 ng/ml, or from about 90 ng/ml to about 100 ng/ml. In certain embodiments, the stem cells are contacted with the at least one BMP active agent in a concentration of between about 20 ng/ml to about 40 ng/ml. In certain embodiments, the stem cells are contacted with the at least one BMP active agent in a concentration of about 30 ng/ml.

In certain embodiments, the stem cells are contacted with the at least one activin protein in a concentration of from about 1 ng/ml to about 100 ng/ml, from about 1 ng/ml to about 20 ng/ml, from about 1 ng/ml to about 15 ng/ml, from about 1 ng/ml to about 10 ng/ml, from about 1 ng/ml to about 5 ng/ml, from about 5 ng/ml to about 10 ng/ml, from about 5 ng/ml to about 15 ng/ml, from about 15 ng/ml to about 25 ng/ml, from about 15 ng/ml to about 20 ng/ml, from about 20 ng/ml to about 30 ng/ml, from about 30 ng/ml to about 40 ng/ml, from about 40 ng/ml to about 50 ng/ml, from about 50 ng/ml to about 60 ng/ml, from about 60 ng/ml to about 70 ng/ml, from about 70 ng/ml to about 80 ng/ml, from about 80 ng/ml to about 90 ng/ml, or from about 90 ng/ml to about 100 ng/ml. In certain embodiments, the stem cells are contacted with the at least one activin protein in a concentration of between about 5 ng/ml to about 10 ng/ml. In certain embodiments, the stem cells are contacted with the at least one activin protein in a concentration of about 7.5 ng/ml.

### 5.2.2. Differentiation of Mesoderm Progenitors to Primitive Hematopoietic Precursors

In certain embodiments, the cells expressing at least one primitive hematopoietic precursor marker are in vitro differentiated from cells expressing at least one mesoderm progenitor marker by contacting cells expressing at least one mesoderm progenitor marker with at least one inhibitor of Wnt signaling (referred to as “Wnt inhibitor”) (e.g., IWP2). In certain embodiments, the stem cells are further contacted with at least one BMP active agent (e.g., a BMP molecule, e.g., BMP4), and at least one activin protein (e.g., Activin A). Non-limiting examples of primitive hematopoietic precursor markers include KDR, CD235A, and combinations thereof.

The term “inhibitor of Wnt signaling” or “Wnt inhibitor” as used herein refers not only to any agent that may act by directly inhibiting the normal function of the Wnt protein, but also to any agent that inhibits the Wnt signaling pathway, and thus recapitulates the function of Wnt. Examples of the Wnt inhibitors include XAV939 (Huang et al. Nature 461:614-620 (2009)), vitamin A (retinoic acid), lithium, flavonoid, Dickkopf1 (Dkk1), insulin-like growth factor-binding protein (IGFBP) (WO2009/131166), and siRNAs against β-catenin.

Non-limiting examples of Wnt inhibitors include XAV939, IWR compounds, IWP compounds (e.g., IWP-2), DKK1 (Dickkopf protein 1), IWR1, peptide (Nile et al peptide (Nile et al Nat Chem Biol. 2018 June; 14(6):582-590), porccupine inhibitors, LGK974, C59, ETC-159, Ant1.4Br/Ant 1.4Cl, niclosamide, apicularen, bafilomycin, G007-LK, G244-LM, pyrvinium, NSC668036, 2,4-diamino-quinazoline, Quercetin, ICG-001, PKF115-584, BC2059, Shizokaol D, derivatives thereof, and combinations thereof. In certain embodiments, the Wnt inhibitor is IWP2.

The Wnt inhibitors can also be those described in WO09155001 and Chen et al., Nat Chem Biol 5:100-7 (2009), which are incorporated by reference in their entireties.

XAV939 is a potent, small molecule inhibitor of tankyrase (TNKS) 1 and 2 with IC₅₀ values of 11 and 4 nM, respectively. Huang et al., Nature 461:614-620 (2009). By inhibiting TNKS activity, XAV939 increases the protein levels of the axin-GSK3β complex and promotes the degradation of β-catenin in SW480 cells. Known Wnt inhibitors also include Dickkopf proteins, secreted Frizzled-related proteins (sFRP), Wnt Inhibitory Factor 1 (WIF-1), and Soggy. Members of the Dickkopf-related protein family (Dkk-1 to -4) are secreted proteins with two cysteine-rich domains, separated by a linker region. Dkk-3 and -4 also have one prokineticin domain. Dkk-1, -2, -3, and -4 function as antagonists of canonical Wnt signaling by binding to LRP5/6, preventing LRP5/6 interaction with Wnt-Frizzled complexes. Dkk-1, -2, -3, and -4 also bind cell surface Kremen-1 or -2 and promote the internalization of LRPS/6. Antagonistic activity of Dkk-3 has not been demonstrated. Dkk proteins have distinct patterns of expression in adult and embryonic tissues and have a wide range of effects on tissue development and morphogenesis.

The Dkk family also includes Soggy, which is homologous to Dkk-3 but not to the other family members. The sFRPs are a family of five Wnt-binding glycoproteins that resemble the membrane-bound Frizzleds. The largest family of Wnt inhibitors, they contain two groups, the first consisting of sFRP1, 2, and 5, and the second including sFRP3 and 4. All are secreted and derived from unique genes, none are alternate splice forms of the Frizzled family. Each sFRP contains an N-terminal cysteine-rich domain (CRO). Other Wnt inhibitors include WIF-1 (Wnt Inhibitory Factor 1), a secreted protein that binds to Wnt proteins and inhibits their activity.

“IWP2” or “Inhibitor of WNT Production-2” refers to IUPAC name N-(6-methyl-1,3-benzothiazol-2-yl)-2-[(4-oxo-3-phenyl-6,7-dihydrothieno[3,2-d]pyrimidin-2-yl)thio]acetamide” with the following formula:

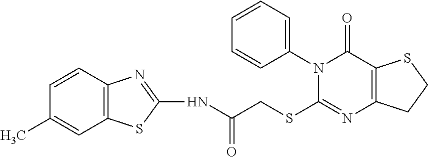 [SMILES: Cc1ccc2nc(NC(=O)CSc3nc4c(c(=O)n3-c3ccccc3)SCC4)sc2c1]

IWP-2 inhibits the WNT pathway (IC₅₀=27 nM) at the level of the pathway activator Porcupine. Porcupine is a membrane-bound acyltransferase that palmitoylates WNT proteins, which leads to WNT secretion and signaling capability.

In certain embodiments, for in vitro differentiation of cells expressing at least one primitive hematopoietic precursor marker, the cells expressing at least one mesoderm progenitor marker are contacted with at least one inhibitor of Wnt signaling for at least about 1 day, or at least about 2 days. In certain embodiments, the cells are contacted with the at least one inhibitor of Wnt signaling for at least about 2 days. In certain embodiments, the cells are contacted with the at least one inhibitor of Wnt signaling for up to about 1 day, up to about 2 days, up to about 3 days, or up to about 4 days. In certain embodiments, the cells are contacted with the at least one inhibitor of Wnt signaling for up to 4 days. In certain embodiments, the cells are contacted with the at least one inhibitor of Wnt signaling for between about 1 day and about 5 days, between about 1 day to about 4 days, between about 1 day and about 3 days, or between about 1 day and about 2 days. In certain embodiments, the cells are contacted with the at least one inhibitor of Wnt signaling for between 2 days and about 5 days. In certain embodiments, the cells are contacted with the at least one inhibitor of Wnt signaling for between 1 day and 3 days. In certain embodiments, the cells are contacted with the at least one inhibitor of Wnt signaling for about 1 day, for about 2 days, for about 3 days, or for about 4 days. In certain embodiments, the cells are contacted with the at least one inhibitor of Wnt signaling for 1 day, 2 days, 3 days, or 4 days. In certain embodiments, the cells are contacted with the at least one inhibitor of Wnt signaling for about 2 days.

In certain embodiments, for in vitro differentiation of cells expressing at least one mesoderm progenitor marker to cells expressing at least one primitive hematopoietic precursor marker, the cells expressing at least one mesoderm progenitor marker can be further contacted with at least one BMP active agent (e.g., BMP4). In certain embodiments, the cells expressing at least one mesoderm progenitor marker are further contacted with at least one activin protein (e.g., Activin A). In certain embodiments, the cells expressing at least one mesoderm progenitor marker are contacted with the Wnt inhibitor(s), BMP active agent(s) and Activin protein(s) concurrently or simultaneously. In certain embodiments, the cells expressing at least one mesoderm progenitor marker are contacted with the at least one inhibitor of Wnt signaling, the at least one BMP active agent (e.g., BMP4), and the at least one activin protein (e.g., Activin A) concurrently for at least about 1 day, at least about 2 days, at least about 3 days, at least about 4 days, or at least about 5 days. In certain embodiments, the cells are contacted with the at least one inhibitor of Wnt signaling, at least one BMP active agent (e.g., BMP4), and at least one activin protein (e.g., Activin A) concurrently for up to about 1 day, up to about 2 days, up to about 3 days, up to about 4 days, or up to about 5 days. In certain embodiments, the cells are contacted with the at least one inhibitor of Wnt signaling for between about 1 day and about 5 days, between about 1 day to about 4 days, between about 1 day and about 3 days, or between about 1 day and about 2 days. In certain embodiments, the cells are contacted with the at least one inhibitor of Wnt signaling, at least one BMP active agent (e.g., BMP4), and at least one activin protein (e.g., Activin A) concurrently for between 1 day and about 3 days. In certain embodiments, the cells are contacted with the at least one inhibitor of Wnt signaling, at least one BMP active agent (e.g., BMP4), and at least one activin protein (e.g., Activin A) concurrently for about 1 day, for about 2 days, for about 3 days, for about 4 days, or for about 5 days. In certain embodiments, the cells are contacted with the at least one inhibitor of Wnt signaling, at least one BMP active agent (e.g., BMP4), and at least one activin protein (e.g., Activin A) concurrently for 1 day, 2 days, 3 days, 4 days, or 5 days. In certain embodiments, the cells are contacted with the at least one inhibitor of Wnt signaling, at least one BMP active agent (e.g., BMP4), and at least one activin protein (e.g., Activin A) concurrently for about 2 days.

In certain embodiments, the cells expressing at least one mesoderm progenitor marker are contacted with the at least one inhibitor of Wnt signaling in a concentration of from about 1 μM to about 100 from about 1 μM to about 20 from about 1 μM to about 15 from about 1 μM to about 10 from about 1 μM to about 5 from about 5 μM to about 10 from about 5 μM to about 15 from about 15 μM to about 20 from about 20 μM to about 30 from about 30 μM to about 40 from about 40 μM to about 50 from about 50 μM to about 60 from about 60 μM to about 70 from about 70 μM to about 80 from about 80 μM to about 90 μM, or from about 90 μM to about 100 μM. In certain embodiments, the cells are contacted with the at least one inhibitor of Wnt signaling in a concentration of from about 1 μM to about 10 μM. In certain embodiments, the cells are contacted with the at least one inhibitor of Wnt signaling in a concentration of from about 1 μM to about 50 μM. In certain embodiments, the ells are contacted with the at least one inhibitor of Wnt signaling in a concentration of about 2 μM. In certain embodiments, the cells are contacted with the at least one inhibitor of Wnt signaling in any one of the above-described concentrations daily, every other day or every two days. In certain embodiments, the cells are contacted with the at least one inhibitor of Wnt signaling in a concentration of about 2 μM daily.

In certain embodiments, the cells expressing at least one mesoderm progenitor marker are contacted with the at least one BMP active agent in a concentration of from about 1 ng/ml to about 100 ng/ml, from about 1 ng/ml to about 20 ng/ml, from about 1 ng/ml to about 15 ng/ml, from about 1 ng/ml to about 10 ng/ml, from about 1 ng/ml to about 5 ng/ml, from about 5 ng/ml to about 10 ng/ml, from about 5 ng/ml to about 15 ng/ml, from about 15 ng/ml to about 25 ng/ml, from about 15 ng/ml to about 20 ng/ml, from about 20 ng/ml to about 30 ng/ml, from about 30 ng/ml to about 40 ng/ml, from about 40 ng/ml to about 50 ng/ml, from about 50 ng/ml to about 60 ng/ml, from about 60 ng/ml to about 70 ng/ml, from about 70 ng/ml to about 80 ng/ml, from about 80 ng/ml to about 90 ng/ml, or from about 90 ng/ml to about 100 ng/ml. In certain embodiments, the stem cells are contacted with the at least one BMP active agent in a concentration of between about 30 ng/ml to about 50 ng/ml. In certain embodiments, the cells are contacted with the at least one BMP active agent in a concentration of about 40 ng/ml. In certain embodiments, the cells are contacted with the at least one BMP active agent in any one of the above-described concentrations daily, every other day or every two days. In certain embodiments, the cells are contacted with the at least one BMP active agent in a concentration of about 40 ng/ml daily.

In certain embodiments, the cells expressing at least one mesoderm progenitor marker are contacted with the at least one activin protein in a concentration of from about 1 ng/ml to about 100 ng/ml, from about 1 ng/ml to about 20 ng/ml, from about 1 ng/ml to about 15 ng/ml, from about 1 ng/ml to about 10 ng/ml, from about 1 ng/ml to about 5 ng/ml, from about 5 ng/ml to about 10 ng/ml, from about 5 ng/ml to about 15 ng/ml, from about 15 ng/ml to about 25 ng/ml, from about 15 ng/ml to about 20 ng/ml, from about 20 ng/ml to about 30 ng/ml, from about 30 ng/ml to about 40 ng/ml, from about 40 ng/ml to about 50 ng/ml, from about 50 ng/ml to about 60 ng/ml, from about 60 ng/ml to about 70 ng/ml, from about 70 ng/ml to about 80 ng/ml, from about 80 ng/ml to about 90 ng/ml, or from about 90 ng/ml to about 100 ng/ml. In certain embodiments, the cells are contacted with the at least one activin protein in a concentration of between about 5 ng/ml to about 15 ng/ml. In certain embodiments, the cells are contacted with the at least one activin protein in a concentration of about 10 ng/ml. In certain embodiments, the cells are contacted with the at least one activin protein in any one of the above-described concentrations daily, every other day or every two days. In certain embodiments, the cells are contacted with the at least one activin protein in a concentration of about 10 ng/ml daily.

5.2.3. Differentiation of Primitive Hematopoietic Precursors To a cell population of EMPs, pMacs, and a Combination Thereof

In certain embodiments, a cell population of differentiated cells are in vitro differentiated from cells expressing at least one primitive hematopoietic precursor marker by contacting the cells with at least one hematopoiesis-promoting cytokine, wherein the differentiated cells are selected from the group consisting of cells expressing at least one erythromyeloid progenitor (EMP) marker, cells expressing at least one pMac maker, and a combination thereof In certain embodiments, the hematopoiesis-promoting cytokines include, but are not limited to, VEGF and activators of FGF signaling. In certain embodiments, the hematopoiesis-promoting cytokine include VEGF and FGF2.

In certain embodiments, the differentiation involves two stages: (1) differentiation of primitive hematopoietic precursors to erythromyeloid progenitors (EMPs); and (2) differentiation of EMPs to pMacs. In certain embodiments, molecules that induce both stages of the differentiation are hematopoiesis-promoting cytokines. For example, hematopoiesis-promoting cytokines involved in the first stage include, but are not limited to, VEGF and activators of FGF signaling. In certain embodiments, the hematopoiesis-promoting cytokine involved in the first stage include VEGF and FGF2. Hematopoiesis-promoting cytokines involved in the second stage include, but are not limited to, stem cell factor (SCF), interleukins (ILs), and Thrombopoietin (TPO). In certain embodiments, the hematopoiesis-promoting cytokines involved in the second stage include SCF, IL-6, IL-3, and TPO.

It was discovered that unsorted samples comprising both the KDR⁺CD235A− and KDR⁺CD235A⁺ cell populations produced hematopoietic cells at nearly the same efficiency as the sorted KDR⁺CD235A⁺ population, which supports that KDR⁺CD235A⁺ cells do not need to be purified for the robust production of hematopoietic cells. In certain embodiments, a cell population comprising cells expressing at least one primitive hematopoietic precursor marker is contacted with the at least one hematopoiesis-promoting cytokine. In certain embodiments, the cell population comprises cells that do not express at least one primitive hematopoietic precursor marker. In certain embodiments, the cells expressing at least one primitive hematopoietic precursor marker are not sorted or isolated from the cell population before the cell population is contacted with the at least one hematopoiesis-promoting cytokine.

Non-limiting examples of EMP markers include Kit, CD41, CD235A, CD43, and combinations thereof. In certain embodiments, the cells expressing at least one EMP marker do not express CD45.

Non-limiting examples of pre-macrophage (pMac) markers include CD45, CSF1R and combinations thereof.

Non-limiting examples of hematopoiesis-promoting cytokines include VEGF, activators of FGF signaling, SCF, interleukins, TPO and combinations thereof. Non-limiting examples of activators of FGF signaling (referred to as “FGF activators”) include FGF1, FGF2, FGF3, FGF4, FGF7, FGF8, FGF10, FGF18, derivatives thereof, and mixtures thereof. In certain embodiments, the at least one FGF activator is FGF2. Non-limiting examples of interleukins include IL-1, IL-2, IL-3, IL-4, IL-5, IL-6, IL-7, IL-8, IL-9, IL-10, IL-11, IL-12, IL-13, and IL-15. In certain embodiments, the interleukin is IL6, IL-3, derivatives thereof, and mixtures thereof In certain embodiments, the at least one hematopoiesis-promoting cytokine is selected from the group consisting of VEGF, FGF2, SCF, IL-6, IL-3, TPO, or a combination thereof. In certain embodiments, the at least one hematopoiesis-promoting cytokine comprises VEGF and FGF2. In certain embodiments, the at least one hematopoiesis-promoting cytokine comprises SCF, IL-6, IL-3, and TPO.

For the in vitro differentiation of cells expressing at least one primitive hematopoietic precursor marker to cells expressing at least one EMP marker, the cells expressing at least one primitive hematopoietic precursor marker are contacted with the at least one hematopoiesis-promoting cytokine for at least about 1 day, at least about 2 days, at least about 3 days, at least about 4 days, or at least about 5 days. In certain embodiments, the cells are contacted with the at least one hematopoiesis-promoting cytokine for at least about 1 day. In certain embodiments, the cells are contacted with the at least one hematopoiesis-promoting cytokine for up to about 1 day, up to about 2 days, up to about 3 days, up to about 4 days, or up to about 5 days, or up to about 10 days. In certain embodiments, the cells are contacted with the at least one hematopoiesis-promoting cytokine for up to about 10 days. In certain embodiments, the cells are contacted with the at least one hematopoiesis-promoting cytokine for between about 1 day and about 5 days, between about 1 day to about 10 days, or between about 5 day and about 10 days. In certain embodiments, the cells are contacted with the at least one hematopoiesis-promoting cytokine for between about 1 day and about 5 days. In certain embodiments, the cells are contacted with the at least one hematopoiesis-promoting cytokine for between 5 day and about 10 days. In certain embodiments, the cells are contacted with the at least one hematopoiesis-promoting cytokine for about 1 day, for about 2 days, for about 3 days, for about 4 days, or for about 5 days, or for about 10 days. In certain embodiments, the cells are contacted with the at least one hematopoiesis-promoting cytokine for about 2 days or about 5 days, or about 10 days. In certain embodiments, the cells are contacted with the at least one hematopoiesis-promoting cytokine for 2 days, for 6 days or for 8 days. In certain embodiments, the at least one hematopoiesis-promoting cytokine comprises VEGF, activators of FGF signaling, or combinations thereof. In certain embodiments, the at least one hematopoiesis-promoting cytokine comprises VEGF and FGF2.

For the in vitro differentiation of cells expressing at least one EMP marker to cells expressing at least one pMac marker, the cells expressing at least one EMP marker are contacted with the at least one hematopoiesis-promoting cytokine for up to about 2 days, up to about 3 days, up to about 4 days, up to about 5 days, or up to about 10 days. In certain embodiments, the cells are contacted with the at least one hematopoiesis-promoting cytokine for up to about 5 days. In certain embodiments, the cells are contacted with the at least one hematopoiesis-promoting cytokine for up to 6 days. In certain embodiments, the cells are contacted with the at least one hematopoiesis-promoting cytokine for at least about 2 days, at least about 3 days, at least about 4 days, at least about 5 days, or at least about 6 days. In certain embodiments, the cells are contacted with the at least one hematopoiesis-promoting cytokine for at least about 2 days. In certain embodiments, the cells are contacted with the at least one hematopoiesis-promoting cytokine for between about 2 days and about 10 days, between about 2 days and about 5 days, or between about 5 days and about 10 days, between about 2 days and about 3 days, between about 3 days and about 6 days, or between about 3 days and about 5 days. In certain embodiments, the cells are contacted with the at least one hematopoiesis-promoting cytokine for between about 2 days and about 5 days, or between about 5 days and about 10 days. In certain embodiments, the cells are contacted with the at least one hematopoiesis-promoting cytokine for about 2 days, about 3 days, about 4 days, about 5 days, or about 6 days. In certain embodiments, the cells are contacted with the at least one hematopoiesis-promoting cytokine for about 5 days. In certain embodiments, the cells are contacted with the at least one hematopoiesis-promoting cytokine for 4 days or 6 days. In certain embodiments, the at least one hematopoiesis-promoting cytokine comprises SCF, interleukins, and TPO. In certain embodiments, the at least one hematopoiesis-promoting cytokine comprises SCF, IL-6, IL-3, and TPO.

In certain embodiments, the cells are contacted with the at least one hematopoiesis-promoting cytokine in a concentration of from about 1 ng/ml to about 400 ng/ml, from about 100 ng/ml to about 400 ng/ml, from about 200 ng/ml to about 400 ng/ml, from about 300 ng/ml to about 400 ng/ml, from about 100 ng/ml to about 300 ng/ml, from about 100 ng/ml to about 200 ng/ml, from about 1 ng/ml to about 00 ng/ml, from about 1 ng/ml to about 20 ng/ml, from about 1 ng/ml to about 15 ng/ml, from about 1 ng/ml to about 10 ng/ml, from about 1 ng/ml to about 5 ng/ml, from about 5 ng/ml to about 10 ng/ml, from about 5 ng/ml to about 15 ng/ml, from about 15 ng/ml to about 25 ng/ml, from about 15 ng/ml to about 20 ng/ml, from about 20 ng/ml to about 30 ng/ml, from about 30 ng/ml to about 40 ng/ml, from about 40 ng/ml to about 50 ng/ml, from about 50 ng/ml to about 60 ng/ml, from about 60 ng/ml to about 70 ng/ml, from about 70 ng/ml to about 80 ng/ml, from about 80 ng/ml to about 90 ng/ml, or from about 90 ng/ml to about 100 ng/ml. In certain embodiments, the cells are contacted with the at least one hematopoiesis-promoting cytokine in a concentration of between about 1 ng/ml to about 10 ng/ml. In certain embodiments, the cells are contacted with the at least one hematopoiesis-promoting cytokine in a concentration of about 5 ng/ml. In certain embodiments, the cells are contacted with the at least one hematopoiesis-promoting cytokine in a concentration of between about 1 ng/ml to about 50 ng/ml. In certain embodiments, the cells are contacted with the at least one hematopoiesis-promoting cytokine in a concentration of about 5 ng/ml, about 10 ng/ml, about 15 ng/ml, about 20 ng/ml, about 30 ng/ml, or about 50 ng/ml. In certain embodiments, the cells are contacted with the at least one hematopoiesis-promoting cytokine in a concentration of between about 50 ng/ml to about 150 ng/ml. In certain embodiments, the cells are contacted with the at least one hematopoiesis-promoting cytokine in a concentration of about 100 ng/ml. In certain embodiments, the cells are contacted with the at least one hematopoiesis-promoting cytokine in a concentration of between about 150 ng/ml to about 250 ng/ml. In certain embodiments, the cells are contacted with the at least one hematopoiesis-promoting cytokine in a concentration of about 200 ng/ml. In certain embodiments, the cells are contacted with the at least one hematopoiesis-promoting cytokine in any one of the above-described concentrations daily, every other day or every two days. In certain embodiments, the cells are contacted with the at least one hematopoiesis-promoting cytokine in a concentration of about 5 ng/ml, about 10 ng/ml, about 15 ng/ml, about 20 ng/ml, about 30 ng/ml, about 50 ng/ml, about 100 ng/ml, or about 200 ng/ml daily.

In certain embodiments, the at least one hematopoiesis-promoting cytokine comprises VEGF, FGF2, or a combination thereof. In certain embodiments, the at least one hematopoiesis-promoting cytokine comprises SCF, IL3, IL-6, TPO, or combinations thereof. In certain embodiments, the VEGF is in a concentration of between about 5 ng/ml to about 50 ng/ml. In certain embodiments, the VEGF is in a concentration of about 15 ng/ml. In certain embodiments, the FGF2 is in a concentration of between about 1 ng/ml to about 50 ng/ml. In certain embodiments, the FGF2 is in a concentration of about 5 ng/ml. In certain embodiments, the SCF is in a concentration of between about 50 ng/ml to about 400 ng/ml. In certain embodiments, the SCF is in a concentration of about 100 ng/ml. In certain embodiments, the SCF is in a concentration of about 200 ng/ml. In certain embodiments, the IL-6 is in a concentration of between about 2 ng/ml to about 200 ng/ml. In certain embodiments, the IL-6 is in a concentration of about 10 ng/ml. In certain embodiments, the IL-6 is in a concentration of about 20 ng/ml. In certain embodiments, the IL-3 is in a concentration of between about 1 ng/ml to about 50 ng/ml. In certain embodiments, the IL-3 is in a concentration of about 30 ng/ml. In certain embodiments, the TPO is in a concentration of between about 3 ng/ml to about 50 ng/ml. In certain embodiments, the TPO is in a concentration of about 30 ng/ml.

5.2.4. Differentiation of the EMPs and/or pMacs to Microglial Cells

In certain embodiments, cells expressing at least one microglial marker are in vitro differentiated from the differentiated cells obtained according to the methods described in Section 5.2.3 (i.e., cells expressing at least one pMac marker and/or cells expressing at least one EMP marker) by culturing the differentiated cells with neurons.

In certain embodiments, cells expressing at least one microglial marker are in vitro differentiated from the differentiated cells obtained according to the methods described in Section 5.2.3 by contacting the differentiated cells with at least one macrophage-promoting cytokine (e.g., to generate cells expressing at least one macrophage markers, e.g., macrophages); and culturing the cells (e.g., macrophages) with neurons. In certain embodiments, the cells expressing at least one macrophage markers (e.g., macrophages) include cells expressing at least one primitive macrophage markers (e.g., primitive macrophages).

In certain embodiments, a pure and synchronized population of cells expressing at least one microglial marker is produced/generated by contacting the cells with at least one macrophage-promoting cytokines and culturing the cells with neurons. In certain embodiments, at least about 85%, at least about 90%, at least about 95%, at least about 99%, or at least about 100% of the cells in the cell population express at least one microglial marker. In certain embodiments, contacting the cells with at least one macrophage-promoting cytokine and then culturing the cells with neurons yields a population of microglial cells with a higher purity as compared to culturing the cells with neurons without contacting the cells with one more macrophage-promoting cytokine.

Non-limiting examples of microglial markers include CX3CR1, PU.1, CD45, IBA1, P2RY12, TMEM119, SALL1, GPR34, C1QA, CD11B, CD68, CD45, and combinations thereof.

Non-limiting examples of macrophage markers include CD11B, DECTIN, CD14, PU.1, CX3CR1, CD45, and combinations thereof.

Non-limiting examples of primitive macrophage markers include CX3CR1, CD11B, and combinations thereof.

Non-limiting examples of neurons include cortical projection neurons, motor neurons, dopaminergic neurons, interneurons, and peripheral sensory neurons.

Non-limiting examples of macrophage-promoting cytokines include M-CSF, IL-34, GM-CSF, IL-3 and combinations thereof. In certain embodiments, the at least one macrophage-promoting cytokine comprises M-CSF and IL-34.

In certain embodiments, the differentiated cells obtained according to the methods described in Section 5.2.3 (i.e., cells expressing at least one pMac marker and/or cells expressing at least one EMP marker) and macrophages are cultured with neurons for at least about 5 days at least about 10 days, or at least about 15 days. In certain embodiments, the differentiated cells and macrophages are cultured with neurons for at least about 5 days, e.g., 4 days. In certain embodiments, the differentiated cells and macrophages are cultured with neurons for up to about 5 days, up to about 10 days, or up to about 15 days. In certain embodiments, the differentiated cells and macrophages are cultured with neurons for up to about 10 days or about 15 days. In certain embodiments, the differentiated cells and macrophages are cultured with neurons for between about 5 days and about 10 days, between about 10 days and about 15 days, or between about 5 days and about 15 days. In certain embodiments, the differentiated cells and macrophages are cultured with neurons for between about 5 days and about 10 days. In certain embodiments, the differentiated cells and macrophages are cultured with neurons for about 5 days, or about 10 days, or about 15 days. In certain embodiments, the differentiated cells and macrophages are cultured with neurons for 4 days, 5 days, 6 days, 7 days, 8 days, 9 days, 10 days, 11 days, 12 days, 13 days, 14 days, or 15 days.

In certain embodiments, the differentiated cells obtained according to the methods described in Section 5.2.3 (i.e., cells expressing at least one pMac marker and/or cells expressing at least one EMP marker) are contacted with at least one macrophage-promoting cytokine for at least about 5 days, at least about 10 days (e.g., at least 9 days or at least 11 days), or at least about 15 days. In certain embodiments, the differentiated cells obtained according to the methods described in Section 5.2.3 (i.e., cells expressing at least one pMac marker and/or cells expressing at least one EMP marker) are contacted with at least one macrophage-promoting cytokine for at least about 5 days or at least about 10 days (e.g., at least 11 days). In certain embodiments, the differentiated cells obtained according to the methods described in Section 5.2.3 (i.e., cells expressing at least one pMac marker and/or cells expressing at least one EMP marker) are contacted with at least one macrophage-promoting cytokine for up to about 5 days, up to about 10 days, or up to about 15 days. In certain embodiments, the differentiated cells obtained according to the methods described in Section 5.2.3 (i.e., cells expressing at least one pMac marker and/or cells expressing at least one EMP marker) are contacted with at least one macrophage-promoting cytokine for up to about 10 days or up to about 15 days. In certain embodiments, the differentiated cells obtained according to the methods described in Section 5.2.3 (i.e., cells expressing at least one pMac marker and/or cells expressing at least one EMP marker) are contacted with at least one macrophage-promoting cytokine between about 5 days and about 15 days, between about 5 days to about 10 days, or between about 10 days and about 15 days. In certain embodiments, the differentiated cells obtained according to the methods described in Section 5.2.3 (i.e., cells expressing at least one pMac marker and/or cells expressing at least one EMP marker) are contacted with at least one macrophage-promoting cytokine between about 5 days and about 10 days or between about 10 days and about 15 days. In certain embodiments, the differentiated cells obtained according to the methods described in Section 5.2.3 (i.e., cells expressing at least one pMac marker and/or cells expressing at least one EMP marker) are contacted with at least one macrophage-promoting cytokine between about 7 days and about 11 days. In certain embodiments, the differentiated cells obtained according to the methods described in Section 5.2.3 (i.e., cells expressing at least one pMac marker and/or cells expressing at least one EMP marker) are contacted with the at least one macrophage-promoting cytokine for about 5 days, or for about 10 days. In certain embodiments, the differentiated cells obtained according to the methods described in Section 5.2.3 (i.e., cells expressing at least one pMac marker and/or cells expressing at least one EMP marker) are contacted with the at least one macrophage-promoting cytokine for 4 days, 5 days, 6 days, 7 days, 8 days, 9 days, 10 days, or 11 days.

In certain embodiments, the differentiated cells obtained according to the methods described in Section 5.2.3 (i.e., cells expressing at least one pMac marker and/or cells expressing at least one EMP marker) are contacted with at least one macrophage-promoting cytokine in a concentration of from about 1 ng/ml to about 250 ng/ml, from about 1 ng/ml to about 100 ng/ml, from about 1 ng/ml to about 100 ng/ml, from about 1 ng/ml to about 20 ng/ml, from about 1 ng/ml to about 15 ng/ml, from about 1 ng/ml to about 10 ng/ml, from about 1 ng/ml to about 5 ng/ml, from about 5 ng/ml to about 10 ng/ml, from about 5 ng/ml to about 15 ng/ml, from about 15 ng/ml to about 25 ng/ml, from about 15 ng/ml to about 20 ng/ml, from about 20 ng/ml to about 50 ng/ml, from about 50 ng/ml to about 100 ng/ml, from about 100 ng/ml to about 150 ng/ml, from about 100 ng/ml to about 200 ng/ml, or from about 150 ng/ml to about 200 ng/ml. In certain embodiments, the differentiated cells are contacted with the at least one macrophage-promoting cytokine in a concentration of from about 5 ng/ml to about 15 ng/ml, from about 15 ng/ml to about 25 ng/ml, from about 40 ng/ml to about 60 ng/ml, or from about 50 ng/ml to about 100 ng/ml, or from about 80 ng/ml to about 100 ng/ml. In certain embodiments, the differentiated cells are contacted with at least one macrophage-promoting cytokine in a concentration of about 10 ng/ml, about 20 ng/ml, about 50 ng/ml or about 100 ng/ml. In certain embodiments, the differentiated cells are contacted with the at least one macrophage-promoting cytokine in any one of the above-described concentrations daily, every other day or every two days. In certain embodiments, the differentiated cells are contacted with the at least one macrophage-promoting cytokine in a concentration of about 10 ng/ml, about 20 ng/ml, about 50 ng/ml or about 100 ng/ml daily. In certain embodiments, the at least one macrophage-promoting cytokine comprises M-CSF, IL-34, or a combination thereof In certain embodiments, M-CSF is in a concentration of between about 1 ng/ml to about 100 ng/ml. In certain embodiments, M-CSF is in a concentration of about 10 ng/ml or about 20 ng/ml. In certain embodiments, IL-34 is in a concentration of between about 5 ng/ml to about 250 ng/ml. In certain embodiments, IL-34 is in a concentration of about 100 ng/ml.

In certain embodiments, the differentiated cells obtained according to the methods described in Section 5.2.3 (i.e., cells expressing at least one pMac marker and/or cells expressing at least one EMP marker) are cultured in a serum-free culture medium that is supplemented with the at least one macrophage-promoting cytokine. In certain embodiments, the serum-free culture medium comprises about 75% IMDM (Iscove's Modified Dulbecco's Medium), about 25% F12 medium. In certain embodiments, the serum-free culture medium further comprises B27, L-glutamine.

In certain embodiments, the differentiated cells obtained according to the methods described in Section 5.2.3 (i.e., cells expressing at least one pMac marker and/or cells expressing at least one EMP marker) are cultured in a culture medium that is supplemented with serum and the at least one macrophage-promoting cytokine.

### Cell Culture Media

In certain embodiments, the above-described inhibitors, activators, and cytokines are added to a cell culture medium comprising the cells disclosed herein. Suitable cell culture media include, but are not limited to, Knockout® Serum Replacement (“KSR”) medium, N2 medium, an Essential 8®/Essential 6® (“E8/E6”) medium, and a Neurobasal (NB) medium (e.g., a NB medium supplemented with N2 and B-27® Supplement). KSR medium, N2 medium, E8/E6 medium and NB medium are commercially available.

KSR medium is a defined, serum-free formulation optimized to grow and maintain undifferentiated hESC cells in culture. The components of a KSR medium are disclosed in WO2011/149762. In certain embodiments, a KSR medium comprises Knockout DMEM, Knockout Serum Replacement, L-Glutamine, Pen/Strep, MEM, and 13-mercaptoethanol. In certain embodiments, 1 liter of KSR medium can comprise 820 mL of Knockout DMEM, 150 mL of Knockout Serum Replacement, 10 mL of 200 mM L-Glutamine, 10 mL of Pen/Strep, 10 mL of 10 mM MEM, and 55 μM of 13-mercaptoethanol.

E8/E6 medium is a feeder-free and xeno-free medium that supports the growth and expansion of human pluripotent stem cells. E8/E6 medium has been proven to support somatic cell reprogramming. In addition, E8/E6 medium can be used as a base for the formulation of custom media for the culture of PSCs. One example E8/E6 medium is described in Chen et al., Nat Methods. 2011 May; 8(5):424-9, which is incorporated by reference in its entirety. One example E8/E6 medium is disclosed in WO15/077648, which is incorporated by reference in its entirety. In certain embodiments, an E8/E6 cell culture medium comprises DMEM/F12, ascorbic acid, selenium, insulin, NaHCO₃, transferrin, FGF2 and TGFβ. The E8/E6 medium differs from a KSR medium in that E8/E6 medium does not include an active BMP or Wnt ingredient.

N2 supplement is a chemically defined, animal-free, supplement used for expansion of undifferentiated neural stem and progenitor cells in culture. N2 Supplement is intended for use with DMEM/F12 medium. The components of a N2 medium are disclosed in WO2011/149762. In certain embodiments, a N2 medium comprises a DMEM/F12 medium supplemented with glucose, sodium bicarbonate, putrescine, progesterone, sodium selenite, transferrin, and insulin. In certain embodiments, 1 liter of a N2 medium comprises 985 ml dist. H₂O with DMEM/F12 powder, 1.55 g of glucose, 2.00 g of sodium bicarbonate, putrescine (100 uL aliquot of 1.61 g dissolved in 100 mL of distilled water), progesterone (20 uL aliquot of 0.032 g dissolved in 100 mL 100% ethanol), sodium selenite (60 uL aliquot of 0.5 mM solution in distilled water), 100 mg of transferrin, and 25 mg of insulin in 10 mL of 5 mM NaOH.

### 5.3 Compositions Comprising Microglial Cells

The presently disclosure provides compositions comprising a population of differentiated microglial cells produced by the in vitro differentiation methods described herewith, for example, in Section 5.2.

Furthermore, the presently disclosed subject matter provides compositions comprising a population of in vitro differentiated cells, wherein at least about 50% (e.g., at least about 55%, at least about 60%, at least about 70%, at least about 75%, at least about 80%, at least about 85%, at least about 90%, at least about 95%, or at least about 99%) of the cells comprised in the population express at least one microglial marker, and wherein less than about 25% (e.g., less than about 20%, less than about 15%, less than about 10%, less than about 5%, less than about 4%, less than about 3%, less than about 2%, less than about 1%, less than about 0.5%, or less than about 0.1%) of the cells comprised in the population express at least one marker selected from the group consisting of stem cells markers, mesoderm progenitor markers, primitive hematopoietic precursor markers, EMP markers, pre-macrophage markers, macrophage markers.

Non-limiting examples of stem cell markers include OCT4, NANOG, SSEA4 and SSEA3.

Non-limiting examples of mesoderm progenitor markers include Brachyury, KDR, and combinations thereof.

Non-limiting examples of primitive hematopoietic precursor markers include KDR, CD235A and combinations thereof.

Non-limiting examples of EMP markers include Kit, CD41, CD235A, CD43, and combinations thereof.

Non-limiting examples of pre-macrophage markers include CD45, CSF1R and combinations thereof.

Non-limiting examples of microglial markers include CX3CR1, PU.1, CD45, IBA1, P2RY12, TMEM119, SALL1, GPR34, C1QA, CD11B, CD68, CD45, and combinations thereof.

Non-limiting examples of macrophage markers include CD11B, DECTIN, CD14, PU.1, CX3CR1, CD45, and combinations thereof.

In certain embodiments, the composition comprises from about 1×10⁴ to about 1×10¹⁰, from about 1×10⁴ to about 1×10⁵, from about 1×10⁵ to about 1×10⁹, from about 1×10⁵ to about 1×10⁶, from about 1×10⁵ to about 1×10⁷, from about 1×10⁶ to about 1×10⁷, from about 1×10⁶ to about 1×10⁸, from about 1×10⁷ to about 1×10⁸, from about 1×10⁸ to about 1×10⁹, from about 1×10⁸ to about 1×10¹⁰, or from about 1×10⁹ to about 1×10¹⁰ of the presently disclosed stem-cell-derived microglial cells. In certain embodiments, the composition comprises from about 1×10⁵ to about 1×10⁷ of the presently disclosed stem-cell-derived microglial cells.

In certain embodiments, said composition is frozen. In certain embodiments, said composition may further comprise at least one cryoprotectant, for example, but not limited to, dimethylsulfoxide (DMSO), glycerol, polyethylene glycol, sucrose, trehalose, dextrose, or a combination thereof.

In certain non-limiting embodiments, the composition further comprises a biocompatible scaffold or matrix, for example, a biocompatible three-dimensional scaffold that facilitates tissue regeneration when the cells are implanted or grafted to a subject. In certain non-limiting embodiments, the biocompatible scaffold comprises extracellular matrix material, synthetic polymers, cytokines, collagen, polypeptides or proteins, polysaccharides including fibronectin, laminin, keratin, fibrin, fibrinogen, hyaluronic acid, heparin sulfate, chondroitin sulfate, agarose or gelatin, and/or hydrogel. (See, e.g., U.S. Publication Nos. 2015/0159135, 2011/0296542, 2009/0123433, and 2008/0268019, the contents of each of which are incorporated by reference in their entireties).

In certain embodiments, the composition is a pharmaceutical composition that comprises a pharmaceutically acceptable carrier. The compositions can be used for regeneration of Peripheral Nervous System (hereafter “PNS”) and/or Central Nervous System (hereafter “CNS”), for preventing and/or treating a microglial cell related disorder.

The presently disclosed subject matter also provides a device comprising the differentiated cells or the composition comprising thereof, as disclosed herein. Non-limiting examples of devices include syringes, fine glass tubes, stereotactic needles and cannulas.

### 5.4 Methods of Treatment

The in vitro differentiated microglial cells can be used for treating neurodegenerative diseases. The present disclosure provides methods for preventing and/or treating a neurodegenerative disease. Non-limiting examples of neurodegenerative diseases include Alzheimer's disease, Amyotrophic Lateral Sclerosis (ALS), Parkinson's disease, schizophrenia, glioblastoma, Huntington's disease, amyotrophic lateral sclerosis, and multiple sclerosis (MS).

In certain embodiment, the method comprises administering to a subject an effective amount of at least one of the followings: (a) a population of differentiated microglial cells described herein; and (b) a composition comprising such differentiated microglial cells.

Furthermore, the presently disclosed subject matter provides for uses of at least one of the followings for preventing and/or treating a neurodegenerative disease: (a) a population of differentiated microglial cells described herein; and (b) a composition comprising such differentiated microglial cells.

The presently disclosed stem-cell-derived microglial cells or a composition comprising thereof, can be administered or provided systemically or directly to a subject. In certain embodiments, the presently disclosed stem-cell-derived microglial cells or a composition comprising thereof, are directly injected into an organ of interest (e.g., an organ affected by a microglial cell defects related disorder). The presently disclosed stem-cell-derived microglial cells or a composition comprising thereof, can be administered (injected) directly to a subject's any part of the body having effective nerves, including, but not limited to, brain.

The presently disclosed stem-cell-derived microglial cells or a composition comprising thereof, can be administered in any physiologically acceptable vehicle. Pharmaceutical compositions comprising a pharmaceutically acceptable carrier and the presently disclosed stem-cell-derived microglial cells, also provided. The presently disclosed stem-cell-derived microglial cells or a composition comprising thereof, or a pharmaceutically acceptable carrier can be administered via localized injection, orthotropic (OT) injection, systemic injection, intravenous injection, or parenteral administration. In certain embodiments, the presently disclosed stem-cell-derived microglial cells or a composition comprising thereof, are administered to a subject via localized injection.

The presently disclosed stem-cell-derived microglial cells or a composition comprising thereof, can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof. Sterile injectable solutions can be prepared by incorporating the compositions of the presently disclosed subject matter, e.g., a composition comprising the presently disclosed stem-cell-derived microglial cells, in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, alum inurn monostearate and gelatin. According to the presently disclosed subject matter, however, any vehicle, diluent, or additive used would have to be compatible with the presently disclosed stem-cell-derived microglial cells or a composition comprising thereof.

Those skilled in the art will recognize that the components of the compositions should be selected to be chemically inert and will not affect the viability or efficacy of the presently disclosed stem-cell-derived microglial cells. This will present no problem to those skilled in chemical and pharmaceutical principles, or problems can be readily avoided by reference to standard texts or by simple experiments (not involving undue experimentation), from this disclosure and the documents cited herein.

In certain non-limiting embodiments, the microglial cells described herein are comprised in a composition that further comprises a biocompatible scaffold or matrix, for example, a biocompatible three-dimensional scaffold that facilitates tissue regeneration when the cells are implanted or grafted to a subject. In certain non-limiting embodiments, the biocompatible scaffold comprises extracellular matrix material, synthetic polymers, cytokines, collagen, polypeptides or proteins, polysaccharides including fibronectin, laminin, keratin, fibrin, fibrinogen, hyaluronic acid, heparin sulfate, chondroitin sulfate, agarose or gelatin, and/or hydrogel. (See, e.g., U.S. Publication Nos. 2015/0159135, 2011/0296542, 2009/0123433, and 2008/0268019, the contents of each of which are incorporated by reference in their entireties).

An “effective amount” (or “therapeutically effective amount”) is an amount sufficient to affect a beneficial or desired clinical result upon treatment. An effective amount can be administered to a subject in at least one doses. In terms of treatment, an effective amount is an amount that is sufficient to palliate, ameliorate, stabilize, reverse or slow the progression of the neurodegenerative disease, or otherwise reduce the pathological consequences of the neurodegenerative disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the cells administered.

In certain embodiments, an effective amount of the presently disclosed stem-cell-derived microglial cells, is an amount sufficient to prevent a neurodegenerative disease, and/or an amount sufficient to treat (e.g., slow the progression of, alleviate and/or reduce the symptoms) of a neurodegenerative disease. The quantity of the presently disclosed stem-cell-derived microglial cells to be administered will vary for the subject being treated. In certain embodiments, from about 1×10⁴ to about 1×10¹⁰ , from about 1×10⁴ to about 1×10⁵, from about 1×10⁵ to about 1×10⁹, from about 1×10⁵ to about 1×10⁶, from about 1×10⁵ to about 1×10⁷, from about 1×10⁶ to about 1×10⁷, from about 1×10⁶ to about 1×10⁸, from about 1×10⁷ to about 1×10⁸, from about 1×10⁸ to about 1×10⁹, from about 1×10⁸ to about 1×10¹⁰, or from about 1×10⁹ to about 1×10¹⁰ the presently disclosed stem-cell-derived microglial cells are administered to a subject. In certain embodiments, from about 1×10⁵ to about 1×10⁷ the presently disclosed stem-cell-derived microglial cells are administered to a subject. The precise determination of what would be considered an effective dose may be based on factors individual to each subject, including their size, age, sex, weight, and condition of the particular subject. Dosages can be readily ascertained by those skilled in the art from this disclosure and the knowledge in the art.

In certain embodiment, the method of preventing and/or treating a neurodegenerative disease comprises administering to a subject an effective amount of a colony-stimulating factor (CSF). Furthermore, the presently disclosed subject matter provides uses of a CSF for preventing and/or treating a neurodegenerative disease.

Non-limiting examples of CSF include granulocyte-macrophage colony-stimulating factor (GM-CSF), M-CSF, IL-34. In certain embodiments, the CSF is GM-CSF, also known as colony-stimulating factor 2 (CSF2).

A CSF is capable of decreasing the complement C3 released from the microglial cells. Therefore, CSFs can be used for preventing and/or treating a neurodegenerative disease (e.g., Alzheimer's disease).

### 5.5 Kits

The presently disclosed subject matter provides kits for inducing differentiation of stem cells. In certain embodiments, the kit comprises (a) at least one inhibitor of Wnt signaling; (b) at least one activator of Wnt signaling; (c) at least one hematopoiesis-promoting cytokine; and (d) neurons. In certain embodiments, the kit further comprises

(e) at least one macrophage-promoting cytokine. In certain embodiments, the kits further comprise (f) instructions for inducing differentiation of the stem cells into cells expressing at least one microglial marker.

In certain embodiments, the instructions comprise contacting the stem cells with the inhibitor(s), activator(s) cytokine(s) and molecule(s) as described by the methods of the present disclosure (see, supra, Section 5.2).

In certain embodiments, the present disclosure provides kits comprising an effective amount of a population of the presently disclosed stem-cell-derived microglial cells or a composition comprising thereof in unit dosage form. In certain embodiments, the kit comprises a sterile container which contains the therapeutic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

In certain embodiments, the kit comprises instructions for administering a population of the presently disclosed stem-cell-derived microglial cells or a composition comprising thereof to a subject suffering from a neurodegenerative disease. The instructions can comprise information about the use of the cells or composition for treating and/or preventing a neurodegenerative disease. In certain embodiments, the instructions comprise at least one of the following: description of the therapeutic agent; dosage schedule and administration for treating or preventing a neurodegenerative disease or symptoms thereof; precautions; warnings; indications; counter-indications; over dosage information; adverse reactions; animal pharmacology; clinical studies; and/or references. The instructions can be printed directly on the container (when present), or as a label applied to the container, or as a separate sheet, pamphlet, card, or folder supplied in or with the container.

### 5.6 Methods of Screening Therapeutic Compounds

The presently disclosed stem-cell-derived microglial cells can be used for modelling a neurodegenerative disease (e.g., Alzheimer's and Amyotrophic Lateral Sclerosis (ALS)).

The presently disclosed stem-cell-derived microglial cells can also serve as a platform to screen for candidate compounds that can overcome disease cellular phenotypes. The capacity of a candidate compound to alleviate a neurodegenerative disease can be determined by assaying the candidate compound's ability to rescue a physiological or cellular defect, which causes a neurodegenerative disease.

In certain embodiments, the method comprises: (a) contacting a population of the presently disclosed microglial cells with a test compound, wherein the microglial cells are derived from stem cells obtained from a subject with the neurodegenerative disease; and (b) measuring functional activity of the microglial cells, wherein a change in the functional activity of the microglial cells indicates that the test compound is likely to be capable of treating a neurodegenerative disease. The change can be a decrease or an increase. In certain embodiments, the change is a decrease. In certain embodiments, the microglial cells are contacted with the test compound for at least about 24 hours (1 day), about 2 days, about 3 days, about 4 days, about 5 days, about 6 days, about 7 days, about 8 days, about 9 days, or about 10 days. In certain embodiments, the microglial cells are contacted with the test compound for at least about 24 hours (1 day).

In certain embodiments, the functional activity of the microglial cells comprises release of complement C3. In certain embodiments, the change is a decrease. Increased complement C3 has been shown in mouse models of Alzheimer's and has been implicated in aberrant pruning of synapses in the disease state. In certain embodiments, a decrease in the complement C3 released from the microglial cells indicates that the test compound is likely to be capable of treating a neurodegenerative disease, e.g., Alzheimer's disease or ALS.

The inventors found that wildtype stem cells-derived microglial cells have a selectivity for pathogenic amyloid-beta-42 over the innocuous amyloid-beta-40. In certain embodiments, the functional activity of the microglial cells comprises amyloid-beta phagocytosis by the microglial cells. In certain embodiments, the change is an increase. In certain embodiments, an increase in amyloid-beta phagocytosis by the microglial cells indicates that the test compound is likely to be capable of treating a neurodegenerative disease, e.g., Alzheimer's disease.

The inventors discovered microglial cells express the highest levels of C9ORF22, a gene that is mutated in an early-onset genetically inherited form of ALS. The inventors also discovered that complement C3 release is increased in C9ORF22 mutant stem cells-derived microglial cells. Activated microglia can induce neurotoxic reactive astrocytes that can induce neurotoxicity of motor neurons. See Liddelow et al., “Neurotoxic reactive astrocytes are induced by activated microglia”, Nature (26 Jan. 2017);541:481-487. In certain embodiments, the method comprises: (a) contacting a test compound with a composition comprising the presently disclosed microglial cells, a population of astrocytes, and a population of neurons ; and (b) measuring neurotoxicity of the neurons, wherein a reduction or decrease in the neurotoxicity of the neurons after the contact with the test compound indicates that the test compound is likely to be capable of treating a neurodegenerative disease. In certain embodiments, the neurons are motor neurons, and the method is used for screening compounds for treating ALS. Non-limiting examples of neurotoxicity of neurons include synapse loss, axonal degeneration, and apoptosis. In certain embodiments, the composition is contacted with the test compound for at least about 24 hours (1 day), about 2 days, about 3 days, about 4 days, about 5 days, about 6 days, about 7 days, about 8 days, about 9 days, or about 10 days. In certain embodiments, the composition are contacted with the test compound for at least about 4 days.

### 6. EXAMPLE

The presently disclosed subject matter will be better understood by reference to the following Example, which is provided as exemplary of the presently disclosed subject matter, and not by way of limitation.

### 6.1 Example 1 Differentiating Microglial from Human Stem Cells

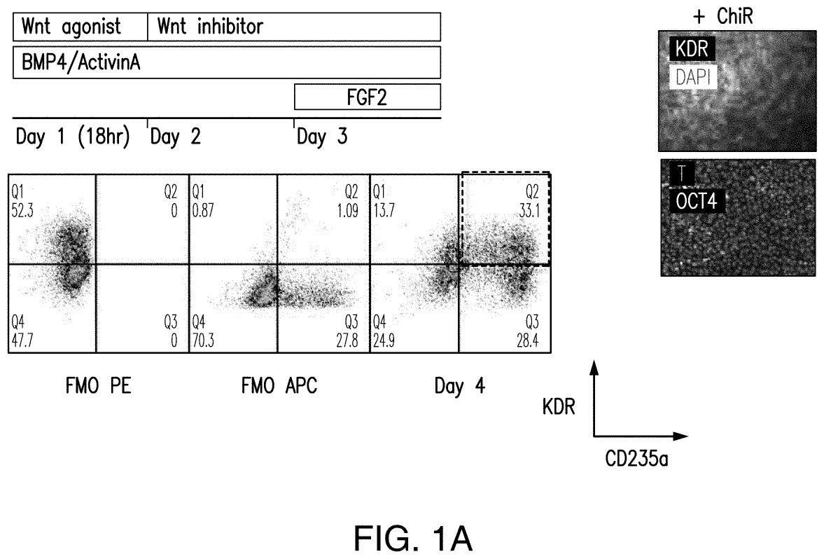

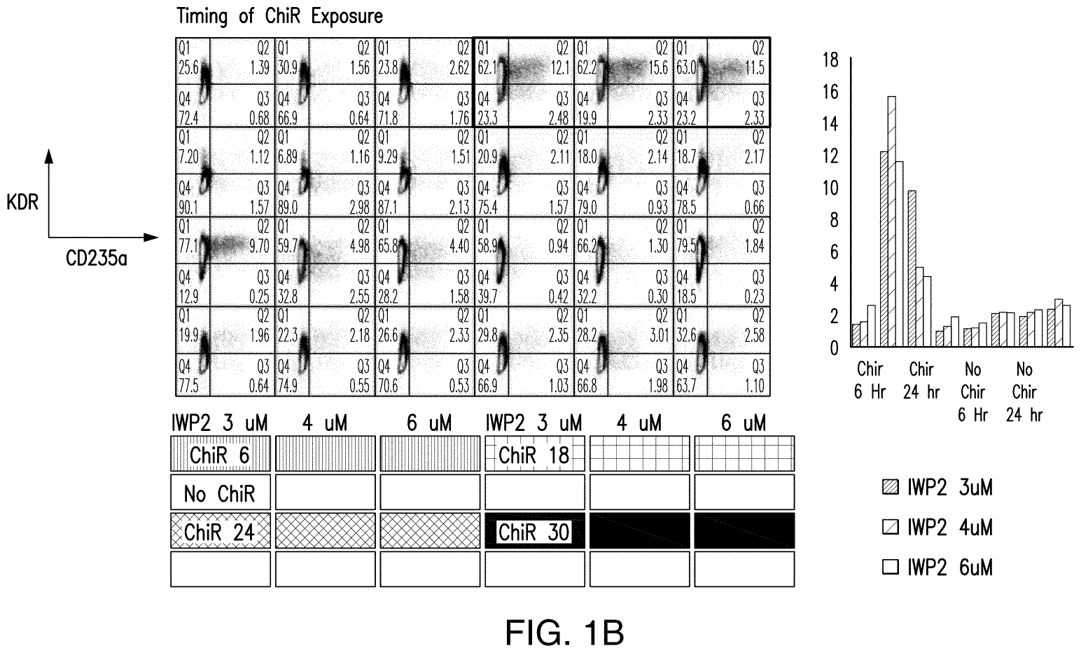

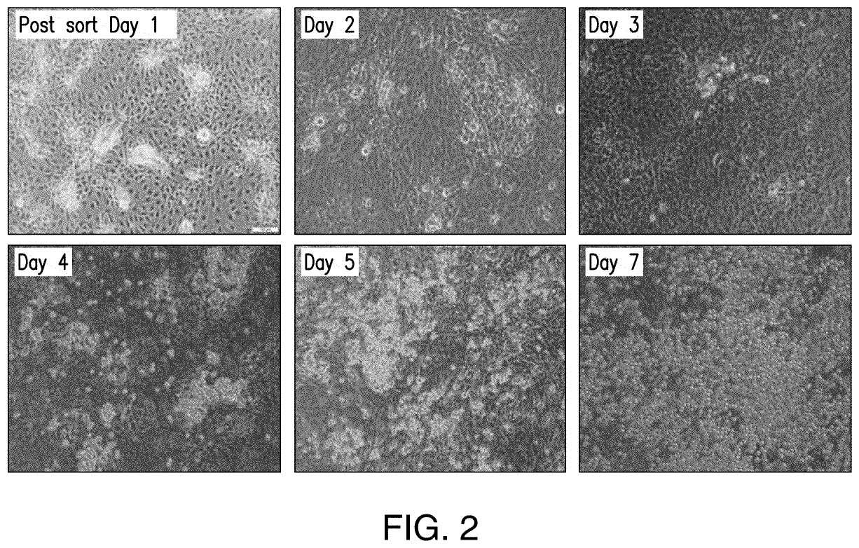

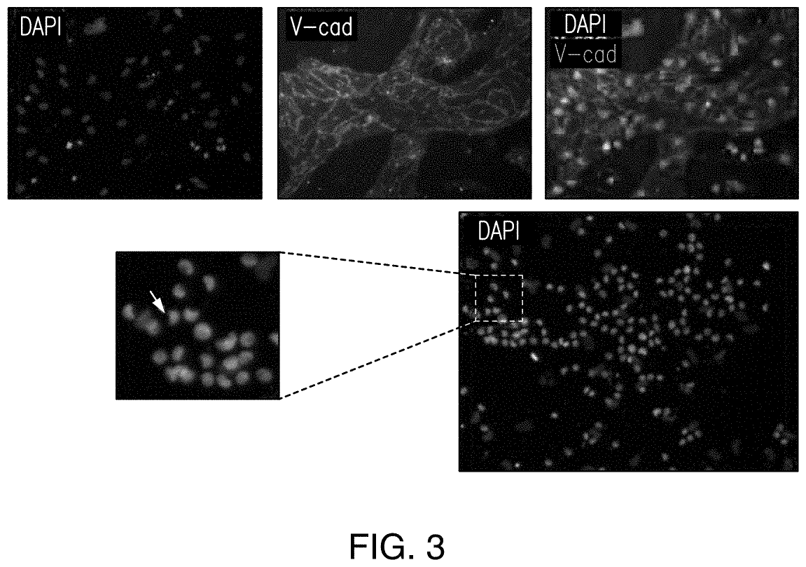

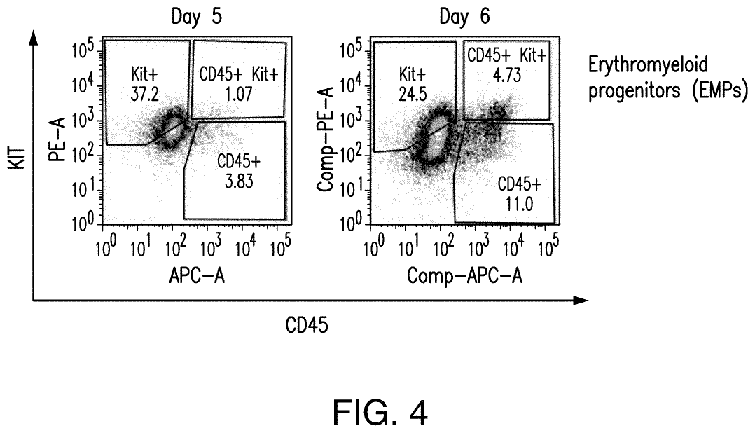

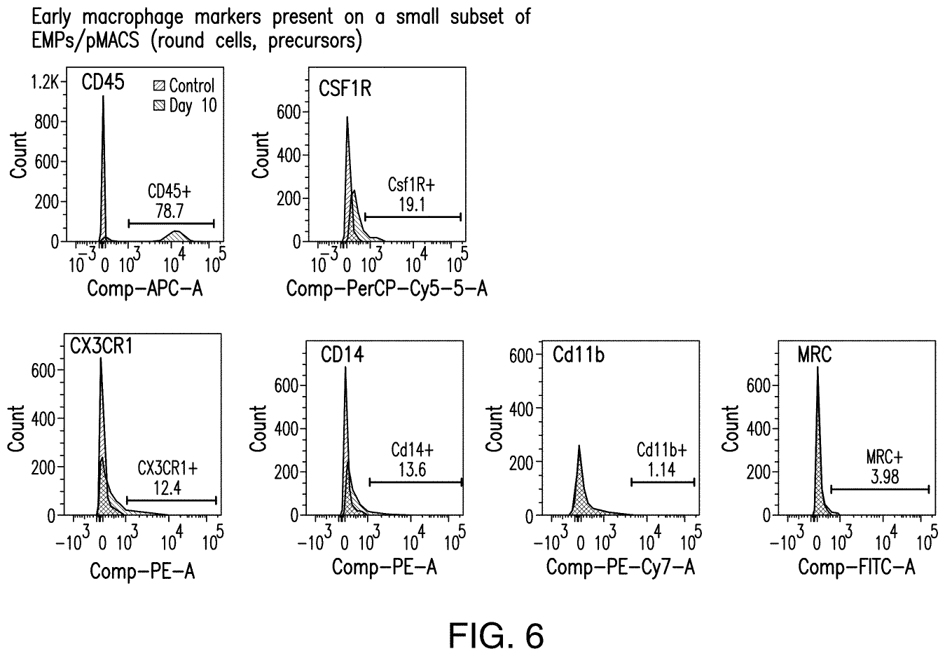

Inhibition of Wnt signaling biases early mesoderm cells from the posterior primitive streak to generate KDR+CD235a+ primitive hematopoietic precursors (Sturgeon et al., Nat. Biotechnol. (2014); 32, 554-561). These precursors will go on to initiate primitive hematopoiesis in the yolk sac, giving rise to early EMPs. In this microglial differentiation scheme, the present example adapted Wnt inhibition to induce primitive hematopoiesis from hPSCs in a monolayer strategy. In particular, the present example showed that eighteen hours of Wnt activation by a Wnt agonist Chir099021 gave rise to early mesoderm, marked by Brachyury (T) by immunofluorescence (FIG. 1A). These cells were also KDR+ and CD235a+ (primitive hematopoietic precursors) by day 4 of differentiation by flow cytometry. The concentration and timing of Chir099021 exposure in the differentiation scheme was optimized, where exposure for 18 hr at 3 uM followed by Wnt inhibition via IWP2 at 3 uM yielded the highest percentage of primitive hematopoietic precursors (KDR+CD235a+) by flow cytometry (FIG. 1B). Next, the present example developed the timeline of hemogenic endothelium and hematopoietic cell. Hemogenic endothelium developed after 1 day of replating the sorted KDR⁺CD235A⁺ primitive hematopoietic precursors. These cells gave rise to hematopoietic cells in suspension over 7 days in culture, gradually becoming more numerous in culture (FIG. 2). In order to validate hemogenic endothelium by VE-cadherin+staining, at 1 day post-sort, the cells were positive for VE-cadherin by immunofluorescence confirming that they were hemogenic endothelium, and the cells in suspension had characteristic small nuclei of hematopoietic cells (FIG. 3). At day 5 after replating the primitive hematopoietic precursors, Kit⁺ cells emerged, which were early erythromyeloid progenitors (EMPs), the precursors to microglia. These cells then gained CD45⁺ by flow cytometry, indicating complete hematopoietic commitment (FIG. 4). In day 8 post sort cultures, where there was a mixed population of EMPs, these cells were identified as microglial and macrophage progenitors and not mature macrophages (FIG. 6). Only a small proportion expressed mature macrophage markers such as CX3CR1, CD14, Cd11b, and MRC. Most cells expressed CD45, however, indicating that they were committed to the hematopoietic lineage. All markers were checked by flow cytometry.

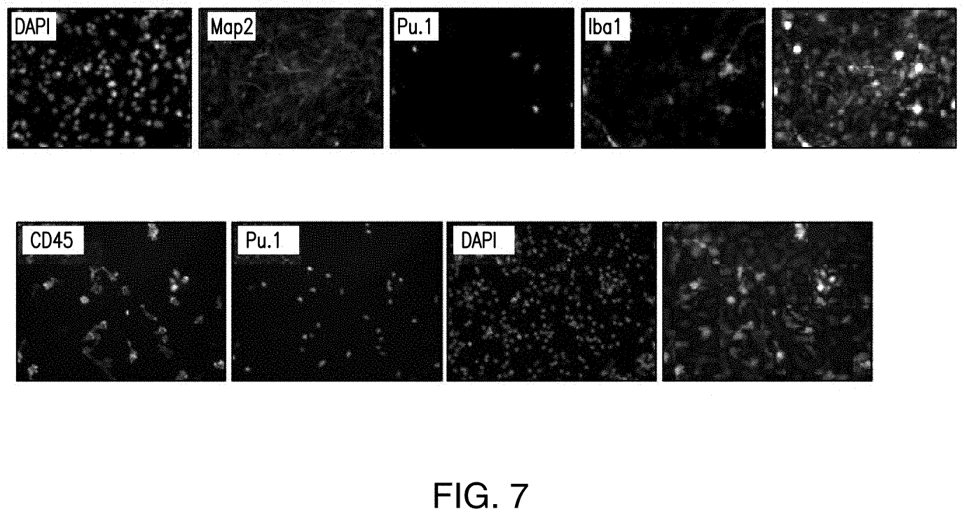

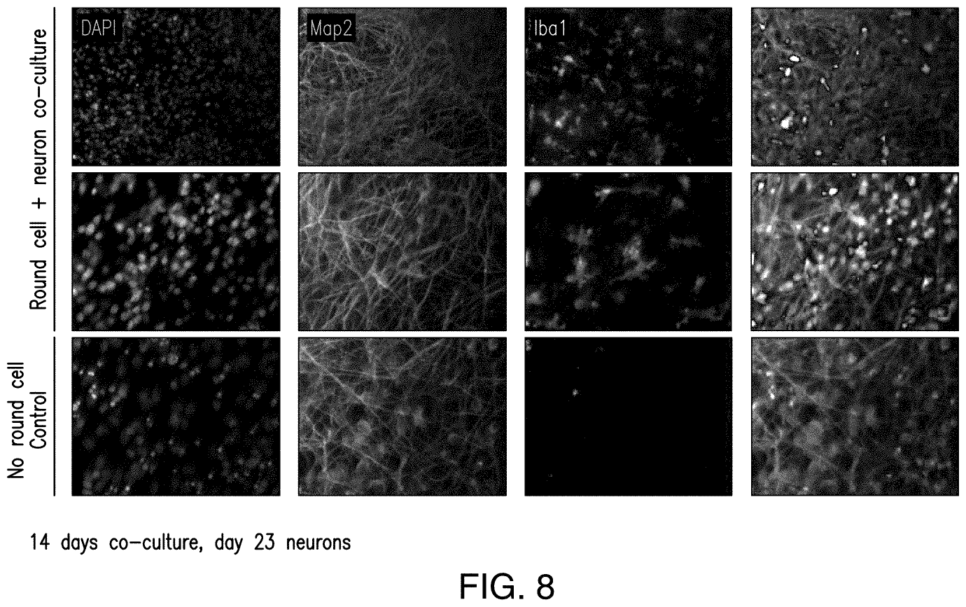

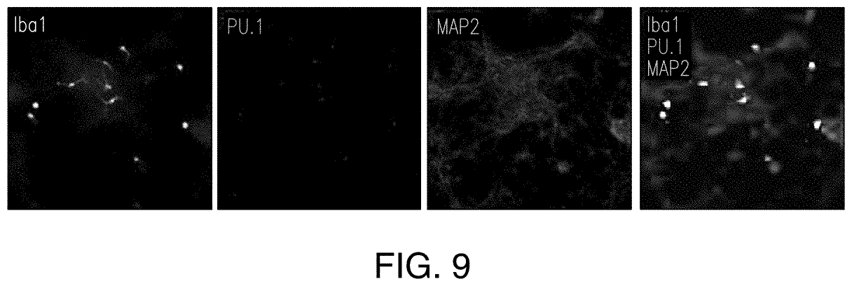

Culturing the cells in suspension at day 8 post-sort, which contained pMacs, with neurons gave rise to early microglial cells by only day 4 of co-culture (FIG. 7). These cells were identified with Iba1+ and Pu.1+ staining. All cells that were CD45⁺ were also Pu.1⁺, indicating that any hematopoietic cell that had persisted in culture (delineated by CD45⁺), was committed to the myeloid/microglial lineage (delineated by PU.1). This indicates that this strategy is highly efficient, by immunofluorescence. The microglial cells (Iba1+ by immunofluorescence) were still persistent in co-culture after 14 days of co-culture with cortical neurons, indicating Iba1+ cells were stable in culture (FIG. 8). The microglial cells (Iba1⁺, PU.1⁺ by immunofluorescence) still persisted in co-culture with neurons after 21 days in culture (FIG. 9).

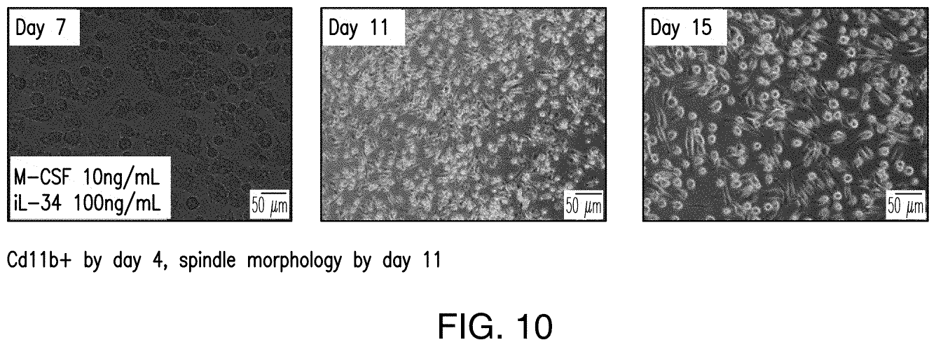

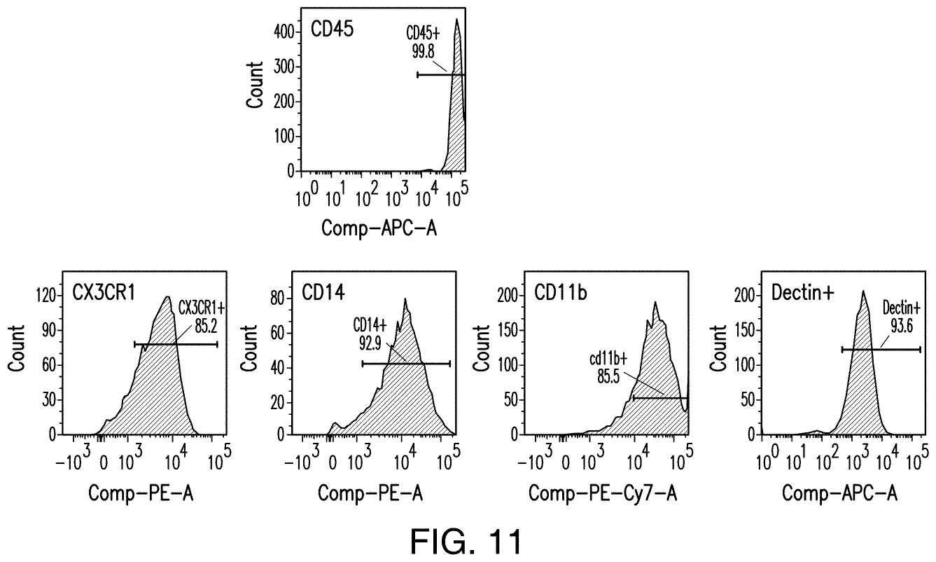

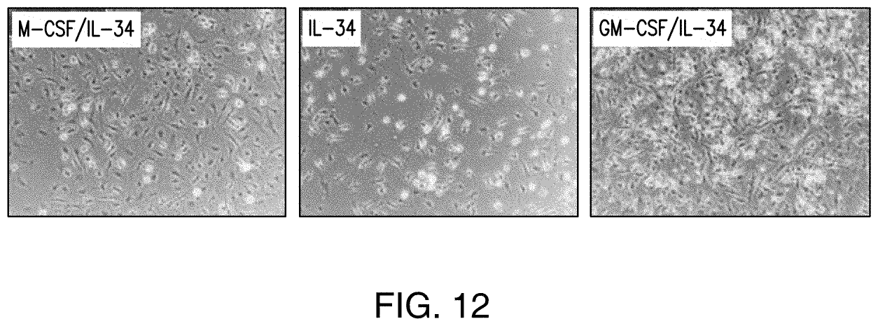

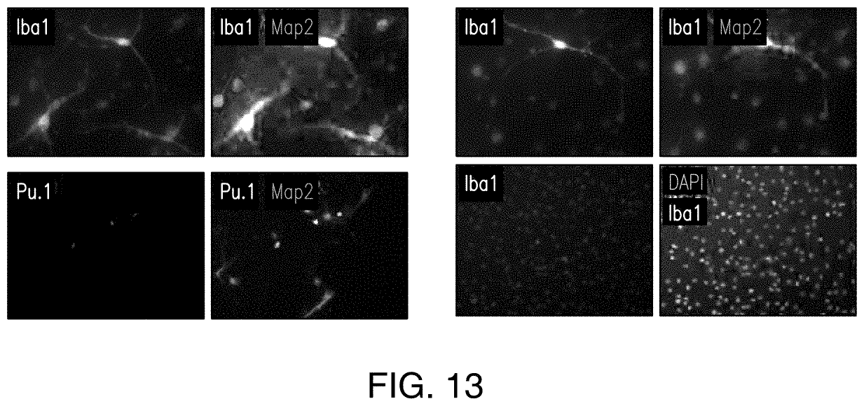

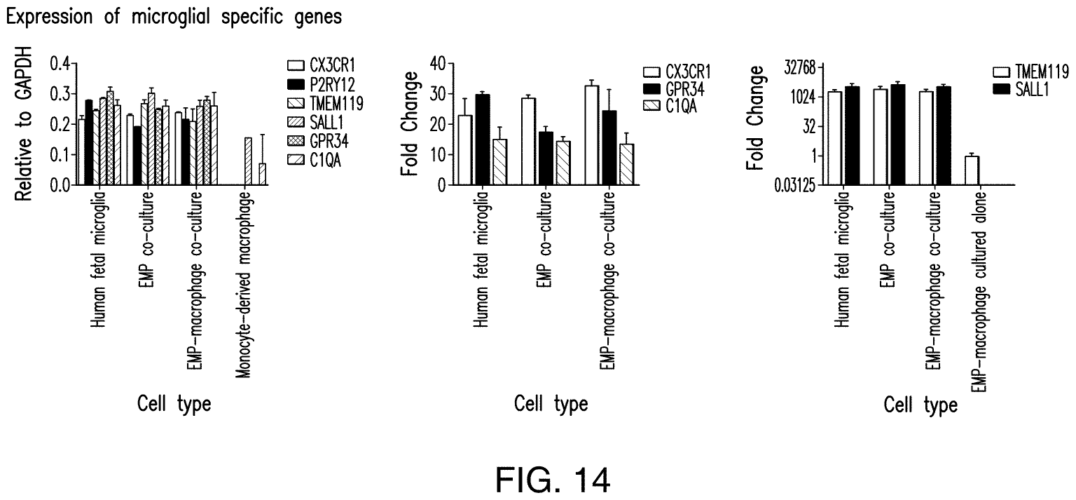

In the second strategy to generate microglia, the EMPs were matured into macrophages with 10% serum and M-CSF and IL-34. They developed the mature macrophage marker Cd11b by day 4 in culture, and the mature spindle morphology by day 11 (FIG. 10). All the cells that persisted in culture in serum and M-CSF and IL-34 after 11 days were CD45⁺, indicating they were all hematopoietic. The majority also were positive for mature macrophage markers such as CX3CR1, CD14, Cd11b, and dectin by flow cytometry analysis (FIG. 11). The pMacs can also be matured into macrophages without serum, using IMDM/F12/N2/B27 in place of serum along with M-CSF and IL-34 (FIG. 12). Addition of M-CSF with IL-34 increased yield by encouraging cell division. Addition of GM-CSF allowed division of the cells as well but the resulting cells appeared more granular and activated. Macrophage cells co-cultured with neurons yielded microglial cells with branching morphology and increased Iba1 immunofluorescence staining (FIG. 13). Iba1 was increased in microglial cells from macrophages, so the cells had transitioned into microglial identity through co-culture with neurons. Co-cultured pMac-derived microglia (EMP co-culture) and pMac-macrophage derived microglia (EMP-macrophage co-culture) share expression of key microglial genes with human fetal microglia (FIG. 14). Quantitative PCR of RNA from the two different strategies to derive microglia shared gene expression of a panel of key microglial genes with RNA from human fetal microglia (commercial source). In contrast, monocyte-derived macrophages, which represented peripheral macrophages, did not express these markers. Noticeably, when the EMP-derived macrophages were cultured alone, they down regulated the key microglial genes TMEM119 and SALL1, indicating that co-culture was necessary to maintain microglial identity.

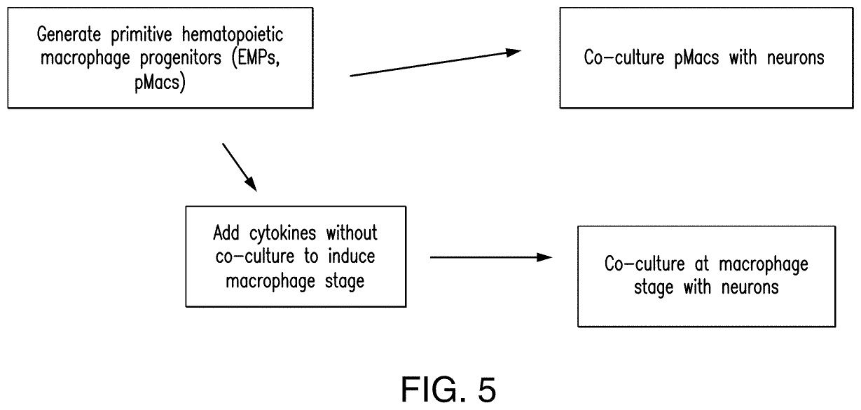

In summary, the present example adapted Wnt inhibition to induce primitive hematopoiesis from hPSCs in a monolayer strategy (FIG. 5). First, the present example patterned the hPSCs into Brachyury⁺ (T⁺) posterior primitive streak early mesoderm cells with Wnt activation by the small molecule CHIR99021. Then, the present example induced these cells to robustly generate KDR+CD235a+ primitive hematopoietic precursors by blocking Wnt signaling by a small molecule inhibitor, IWP2. The present example determined that treatment with CHIR99021 for 18 hours exactly followed by IWP2 treatment is the only condition in which a sizable proportion of KDR⁺CD235A⁺ cells develop. While previously published papers have used Wnt activation to induce mesodermal cells, they do not then try to pattern this mesoderm towards KDR⁺CD235A⁺ precursors, and therefore are not restricted to the 18-hour window of treatment beyond which the present example have determined that the potential to form this population is greatly reduced. Furthermore, in the previous study which identified that KDR⁺CD235A⁺ precursors are primitive hematopoietic precursor cells, the authors allow this population to arise through an embryoid body method in which endogenous signaling plays a greater role, and thus they can use only Wnt inhibition alone (Sturgeon et al., Nat. Biotechnol. (2014); 32, 554-561). Therefore, the present example induced KDR⁺CD235A⁺ primitive hematopoietic precursors from hPSCs through first using an 18-hour treatment of Wnt activation to induce mesoderm followed by Wnt inhibition, and is a novel method in a defined, monolayer system.

Next, the present example drove KDR⁺CD235A⁺ primitive hematopoietic precursors to form EMPs by addition of a cocktail of hematopoiesis-promoting cytokines: first VEGF/FGF2 to induce VE-cadherin+ hemogenic endothelium, then SCF, IL-6, IL-3, and TPO to encourage transition to CD34+ followed by CD45⁺ hematopoietic cells in a stepwise fashion over 8 days. Floating CD45+ pMacs were then harvested from the day 8 cultures for co-culture with neurons for direct transition into microglia, or maturation into macrophages and then co-cultured with neurons for transition into microglia. The first method is particularly novel in that it is the only method that shows that microglial cells can develop directly from the earliest pMacs within 4 days upon co-culture with neurons, like the in vivo development in the mouse (Mass et al., Science (2016); 353(6304) aaf4238).

Existing strategies to generate human microglial cells in vitro do not follow this developmental paradigm, failing to first pattern towards primitive hematopoietic precursors. These strategies either start from peripheral monocytes (Noto et al., Neuropathol. Appl. Neurobiol. (2014); 40, 697-713; Ohgidani et al., Sci. Rep. (2014); 4, 4957), which arise from definitive hematopoiesis and therefore do not give rise to microglial cells in vivo or use embryoid body approaches which are not pre-patterned for primitive hematopoiesis and are black boxes with respect to developmental cell fate decisions (Etemad et al., Neurosci. (2012); 209, 79-89; Hinze et al., Inflamm. (2012); 9, 12; Lachmann et al., Stem Cell Reports (2015);4, 282-296; Noto et al., Neuropathol. Appl. Neurobiol. (2014); 40, 697-713; Ohgidani et al., Sci. Rep. (2014); 4, 4957; van Wilgenburg et al., PLoS One (2013); 8.; Muffat et al., Nature Medicine (2016); 22(11), 1358-1367; Bahrini et al., Sci. Rep. (2015);5, 7989). Of the monolayer protocols to derive microglial-like cells from hPSCs (Abud et al., Neuron, (2017); 94(2), 278-293; Takata et al., Immunity. (2017); 47(1), 183-198), the cells are again not patterned towards primitive hematopoietic KDR⁺CD235A⁺ cells and therefore there is no way to determine if these strategies go through early EMPs, which are the true precursors to microglia, or late EMPs, which are a precursor of definitive hematopoiesis. Moreover, all these strategies first generate fully differentiated macrophages from the CD45⁺ pMacs before co-culture with neurons (if at all co-cultured), and therefore do not represent the in vivo developmental trajectory of precursor cells (pMacs) first colonizing the brain to develop into tissue-resident microglia (Mass et al., Science (2016); 353(6304), aaf4238). The present strategy is novel in that it uses a step-wise developmental paradigm that recapitulates yolk sac primitive hematopoiesis, isolates pMacs before maturation into macrophages, and cultures these cells in an in vitro neural niche to generate bona fide human microglial cells in as little as 16 days.

An exemplary protocol for differentiating microglial from human stem cells is shown below:

Stage I: pMac production (serum-free)
1) DAY 0: Plate ES cells at 60,000 cells/cm\hat{ }²+Y in ABC* media in either 24-well or 6-well tissue culture plates coated with 1:30 matrigel.
2) DAY 1: After 18 hr, change media to ABi medium. Cells should be T+(Brachyury+).
3) DAY 2: Change media to ABi+FGF2.
4) DAY 3: Split and plate cells at 60,000 cells/cm\hat{ }² in VEGF/FGF2 media+Y on matrigel. At least 30% of cells should be KDR⁺CD235A⁺.
5) DAY 4: Take out Y-drug and leave in VEGF/FGF2.
6) DAY 5: Add Cyto 1 media. 10-20% of cells should be Kit⁺ (EMPs).
7) DAY 6: Check to see if there are small round cell colonies forming.
8) DAY 7: Add Cyto 2 media.
9) DAY 8: Round cells should be confluent. Collect the cells in suspension and spin down. Cultures should contain a VE-cadherin+hemogenic endothelium, and a suspension of round cells that are Kit+ (EMPs) or Kit⁻CD45⁺CSF1R⁺ (pMacs). Stage IIa. Direct conversion to microglial cells with neuronal co-culture (serum-free)
1) Collect only the suspension cells from the day 8 cultures and spin down.
2) Plate the suspension cells with neurons at a 1:5 ratio in microglial medium #1.
3) Change media every other day.
   After day 4, many Pu.1⁺CD45⁺, Iba1⁺, CX3CR1⁺ microglial cells should already present in the cultures.
4) Cells should be ready to be assayed after 10-14 days in co-culture.
   Microglial cells when sorted by CX3CR1⁺ should be P2RY12⁺, Tmem119⁺, Sal11⁺, GPR34⁺, and C1QA⁺.

Stage IIb. Differentiation into Macrophage Cells, then Co-Culture with Neurons
1) Plate the suspension cells 1:2 in RPMI media on TC-treated plastic.
2) Change the media every other day (M-W-F), can leave in the media over the weekend.
3) After 11 days, cells should look spindly and granular. Accutase cells for 10 min, and plate with neurons at 1:5 ratio.

Cells should be Cd11b⁺, Dectin⁺, CD14⁺ (mature macrophages).
4) Culture with neurons in microglial co-culture medium and assay after 10-14 days.

Table 1 provides the concentrations and concentration ranges of certain components in the cell culture medium used in the present example.

TABLE 1

| ABC | E8 or E8 flex base | Low | High |
|---|---|---|---|
| Activin A | 7.5 ng/mL | 1 ng/mL | 10 ng/mL |
| BMP4 | 30 ng/mL | 10 ng/mL | 100 ng/mL |
| ChiR | 3 μM | 1 μM | 6 μM |
| Abi | E6 Base | Low | High |
| Activin A | 10 ng/mL | 1 ng/mL | 10 ng/mL |
| BMP4 | 40 ng/mL | 10 ng/mL | 100 ng/mL |
| IWP2 | 2 μM | 1 μM | 10 μM |
| Abi + FGF2 | E6 Base | Low | High |
| Activin A | 10 ng/mL | 1 ng/mL | 10 ng/mL |
| BMP4 | 40 ng/mL | 10 ng/mL | 100 ng/mL |
| IWP2 | 2 μM | 1 μM | 10 μM |
| FGF2 | 20 ng/mL | 10 ng/mL | 50 ng/mL |
| VEGF/FGF2 | E6 Base | Low | High |
| VEGF | 15 ng/mL | 5 ng/mL | 50 ng/mL |
| FGF2 | 5 ng/mL | 1 ng/mL | 50 ng/mL |
| Cyto 1 | E6 Base | Low | High |
| VEGF | 15 ng/mL | 5 ng/mL | 50 ng/mL |
| FGF2 | 5 ng/mL | 1 ng/mL | 50 ng/mL |
| SCF | 200 ng/mL | 50 ng/mL | 400 ng/mL |
| IL-6 | 20 ng/mL | 2 ng/mL | 200 ng/mL |
| Cyto 2 | E6 Base | Low | High |
| SCF | 100 ng/mL | 50 ng/mL | 400 ng/mL |
| IL-6 | 10 ng/mL | 2 ng/mL | 200 ng/mL |
| TPO | 30 ng/mL | 3 ng/mL | 50 ng/mL |
| iL-3 | 30 ng/mL | 1 ng/mL | 50 ng/mL |
| RPMI | RPMI +10% non hyclone FBS +L-glut, P/S | | |
| M-CSF | 10 ng/mL | 1 ng/mL | 100 ng/mL |
| iL-34 | 100 ng/mL | 10 ng/mL | 200 ng/mL |
| Microglia | Neurobasal base +B27, L-glut, | Low (ng/mL) | High (ng/mL) |
| Medium 1 | P/S (ng/mL) | | |
| M-CSF | 10 ng/mL | 1 ng/mL | 100 ng/mL |
| iL-34 | 100 ng/mL | 5 ng/mL | 250 ng/mL |
| Microglia | IMDM 75% F12 25% + N2, | Low (ng/mL) | High (ng/mL) |
| co-culture | B27, L-glut, P/S base (ng/mL) | | |
| IGF1 | 50 ng/mL | 5 ng/mL | 100 ng/mL |
| iL-34 | 100 ng/mL | 5 ng/mL | 250 ng/mL |
| M-CSF | 10 ng/mL | 1 ng/mL | 100 ng/mL |

### 6.2 Example 2: Application of the Generated Microglial Cells

hPSC-derived microglia derived through the present developmental strategy will be useful for interrogating interactions between microglia and neurons in vitro during disease modeling. These mixed cultures can be utilized for multi-cell type drug screens where cellular exchanges are targeted rather than individual cell types alone (Hoing et al., Cell Stem Cell (2012);11, 620-632; Schwartz et al., Proc. Natl. Acad. Sci. U.S.A. (2015); 112(40), 12516-21).

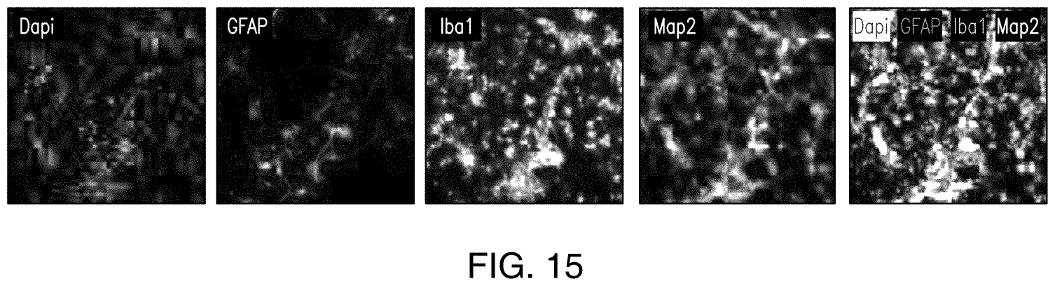

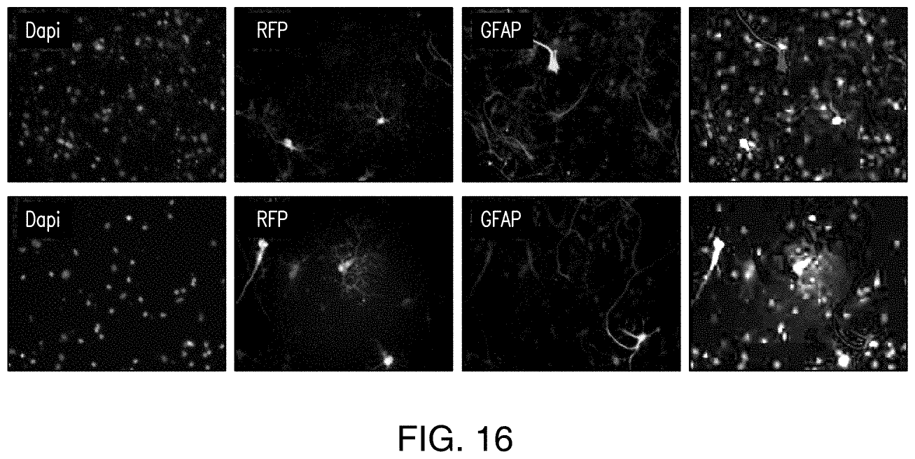

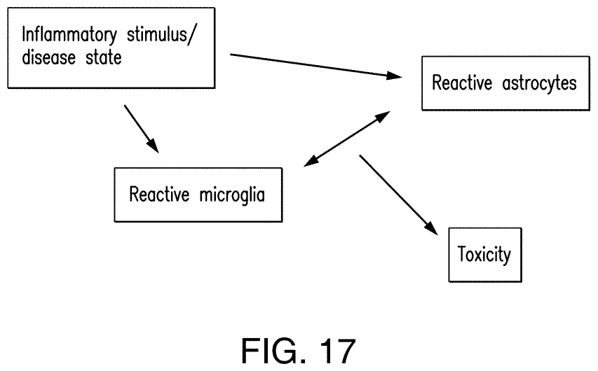

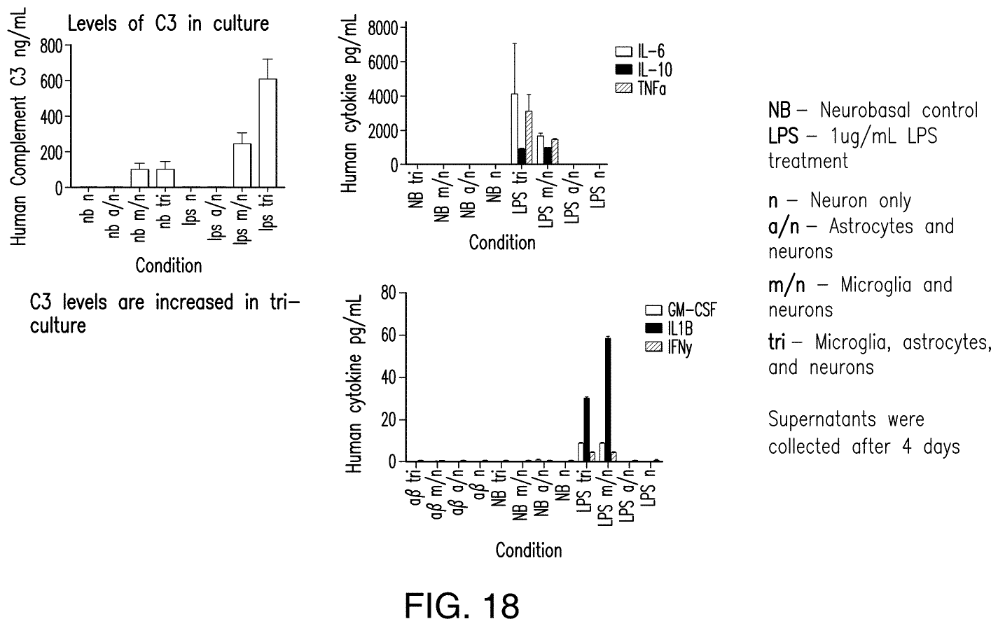

The microglial cells that are derived from the present disclosure can be co-cultured with astrocytes to build a system containing three components of the CNS: neurons, microglia, and astrocytes. Microglia were tagged in RFP, astrocytes were GFAP+by immunofluorescence (FIGS. 15 and 16). Tri-culture system can be used to study interactions between cell types. Inflammatory stimuli, or a disease state inducing an inflammatory stimulus, affects both microglia and astrocytes which have crosstalk (FIG. 17). This crosstalk is a feedback or feedforward loop that can then lead to toxicity to neurons. This interaction can be studied using the presently disclosed hPSC-derived microglia in tri-culture with hPSC-derived astrocytes and neurons to examine a completely human system in vitro. LPS stimulation in tri-culture led to reactive cytokine release (FIG. 18). 1 ug/mL of LPS was added to cells in tri-culture, containing microglia, astrocytes, and neurons, or to cultures containing only microglia and neurons, or only astrocytes and neurons, or only neurons. Only cultures containing microglia responded to LPS, since they were the only cell type expressing the receptor to LPS (TLR4), however in tri-culture, there was an increased release of C3 when compared to a microglia and neurons only culture. The effect may be attributed to reactive cytokines from activated microglia feeding back to astrocytes causing their reactivity and release of astrocytic C3. LPS stimulated tri and microglia/neuron only cultures also secreted other reactive cytokines, including IL-6, TNFα, GM-CSF, IL1B, and IFNγ. Cytokines were measured via ELISA.

As microglia are implicated in the pathogenesis of many diseases, both neurodevelopmental and neurodegenerative (a few examples being schizophrenia, Alzheimer's, Parkinson's, glioblastoma), this offers a powerful tool. Additionally, the present microglial cells might also be used for transplant into the patient brain for treatment of various diseases.

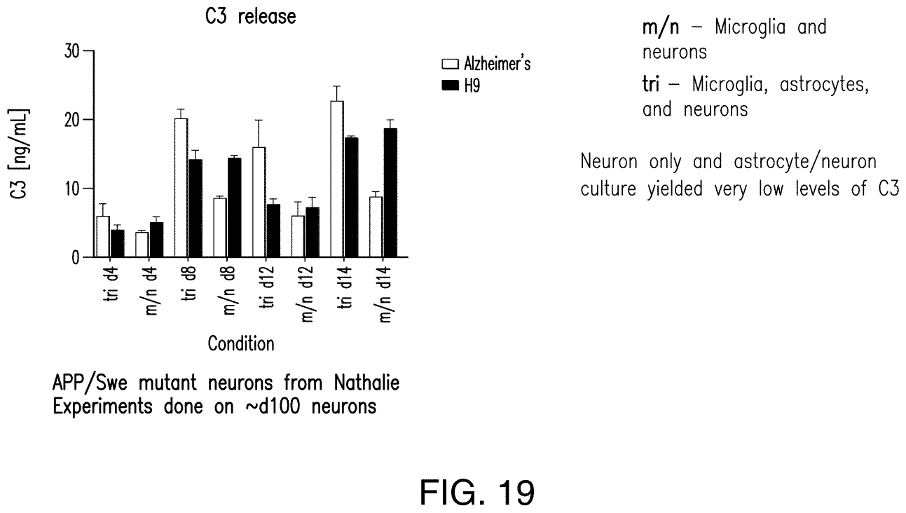

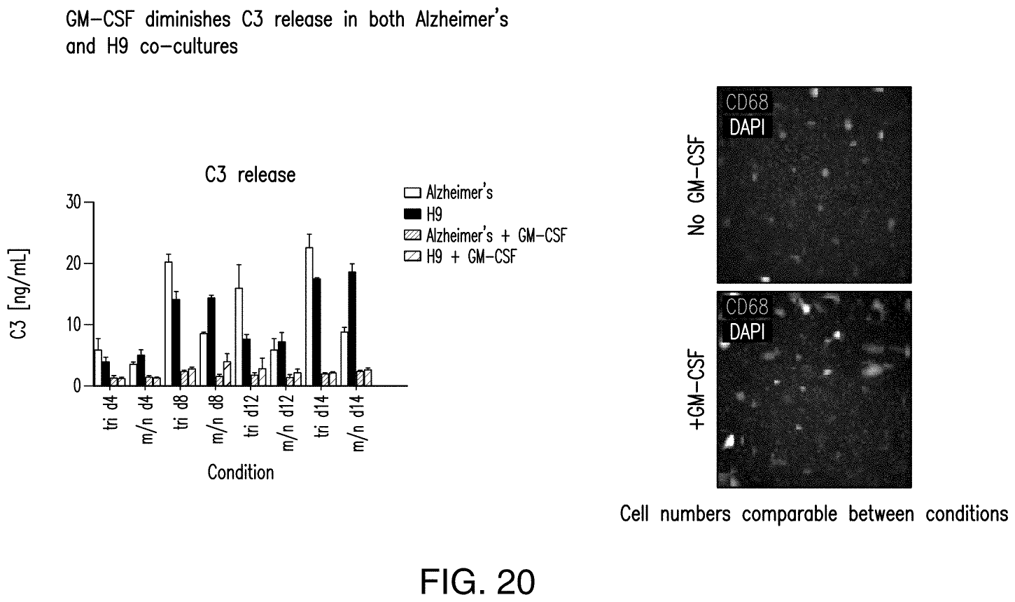

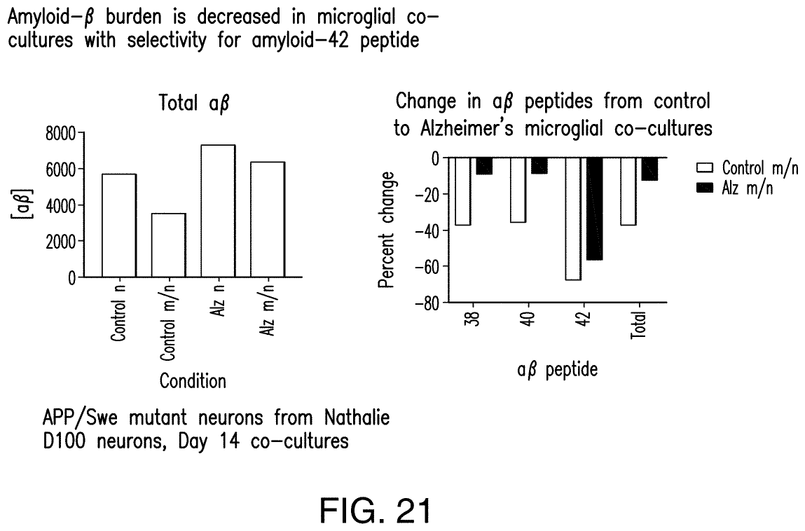

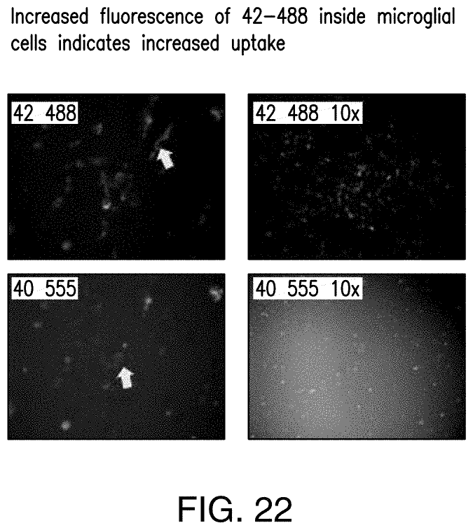

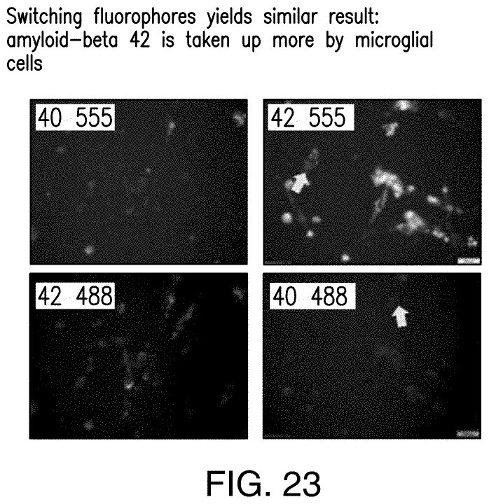

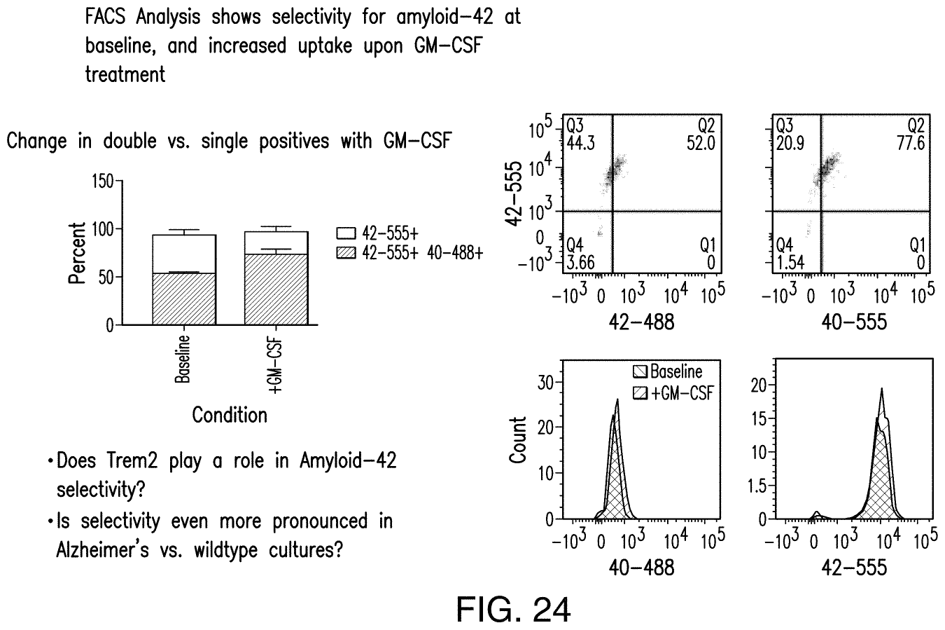

hPSC-derived microglia were used to model two neurodegenerative diseases, Alzheimer's and Amyotrophic Lateral Sclerosis (ALS). Mixed cultures of hPSC-derived microglia and Alzheimer's iPSC-derived neurons allowed to observe microglial activation in the disease culture, particularly with respect to increased complement C3 release. The present example found that tri-cultures with Alzheimer's neurons showed C3 potentiation and increased C3 release when compared to H9 control (FIG. 19). Tri-cultures co-cultured with APP/SWe mutant neurons, a genetic model for familial Alzheimer's disease, showed increased C3 when compared to microglia and neuron only cultures, and the levels were increased when compared to cultures in which the neurons were derived from an H9 control embryonic stem cell line. In contrast, the C3 levels did not increase in the tri-culture as compared to the microglia/neuron culture in the H9 control, indicating that C3 potentiation did not occur without the disease stimulus. C3 was measured via ELISA. Additionally, GM-C SF diminished the C3 release in all cultures, both Alzheimer's and control (FIG. 20). The cell numbers were also comparable between the GM-CSF added conditions and control conditions via immunofluorescence, indicating this effect was not due to fewer microglial cells. Amyloid-β burden was decreased in microglial co-cultures with selectivity for amyloid-42 peptide (FIG. 21). Co-culture of microglial cells with Alzheimer's neurons showed decreased total amyloid beta via ELISA, particularly of the amyloid-beta 42 peptide when compared to the 40 and 38 peptides. Increased fluorescence of 42-488 inside microglial cells indicated increased uptake (FIG. 22). To assay whether microglial cells phagocytose amyloid-beta, a fluorescently tagged amyloid-beta 42 peptide was used with alexa fluor 4888 and found that after 2 hours, the majority of microglial cells contained amyloid beta 42 within them via immunofluorescence. Amyloid-beta 40, on the other hand (tagged via 555), was not found as brightly within the microglial cells, indicating that it was not phagocytosed as efficiently. This demonstrated a selectivity for amyloid beta 42 by microglial cells. Switching fluorophores yielded similar result: amyloid-beta 42 was taken up more by microglial cells (FIG. 23). The fluorophores representing amyloid 42 and 40 were switched to make sure the effect of increased 42 brightness within cells was not due to a technical fluorophore brightness effect. Even with the switched fluorophores, in this experiment 42 was tagged to 555 and 40 to 488, there was more 42 brightness within microglial cells via immunofluorescence, corroborating the previous results that microglial cells phagocytosed amyloid-beta 42 selectively. FACS Analysis showed selectivity for amyloid-42 at baseline, and increased uptake upon GM-CSF treatment (FIG. 24). GM-CSF treatment increased the phagocytosis of amyloid-beta 42 as well as 40 in microglial cells, and the number of cells with the amyloid-beta peptides within them were quantified using flow cytometry.

In summary, increased complement C3 has been shown in mouse models of Alzheimer's and has been implicated in aberrant pruning of synapses in the disease state. The present example interrogates cytokines that can dampen down this increase in C3 from microglia in the present co-cultures and have identified GM-CSF as a potential candidate. The present example also studied amyloid-beta phagocytosis by hPSC-derived microglial cells. The present example found that that wildtype hPSC-derived microglial cells have a selectivity for pathogenic amyloid-beta-42 over the innocuous amyloid-beta-40, and the present example generated CRISPR knockouts of the microglial receptor TREM2 to see if it is involved with this selectivity. The present example plans on doing a CRISPR screen of candidate immune genes and cell surface receptors to determine which genes and pathways are most closely involved in amyloid-beta phagocytosis.

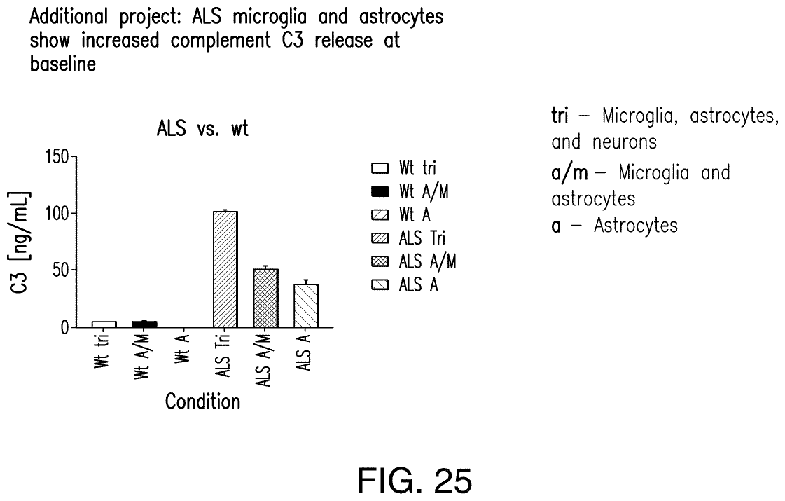

The present second disease model using the hPSC-derived microglia is ALS. The present example found that ALS microglia and astrocytes showed increased complement C3 release at baseline (FIG. 25). SOD1 mutant iPSC-derived ALS astrocytes and microglia were cultured alone or together, and exhibited higher levels of C3 vs. isogenic, wildtype control cell line derived astrocytes or microglia quantified via ELISA. This indicates that C3 reactivity is not unique to Alzheimer's and the presently disclosed system can be used to study other neurodegenerative diseases, where a loop between microglia, astrocytes, and neurons likely also exists.

The present example noticed that microglial cells expressed the highest levels of C9ORF22, a gene which was mutated in an early-onset genetically inherited form of ALS. The present example also noticed that complement C3 release was increased in C9ORF22 mutant iPSC-derived microglial cells, and the present example tested whether these cells, in conjunction with iPSC-derived astrocytes, were neurotoxic to iPSC-derived motor neurons. The present example plans on doing a drug screen using a tri-culture system of the present iPSC-derived microglia, astrocytes, and neurons in an ALS model to find candidates that can rescue this neurotoxicity by acting on a system of neuroinflammation rather than on neurons alone.

### 6.3 Example 3: Fully Defined Human PSC-Derived Microglia and Tri-Culture System Reveals Cell Type Specific Potentiation of Complement Production in a Model of Alzheimer's Disease

Aberrant inflammation in the central nervous system (CNS) has been implicated as a major player in the pathogenesis of human neurodegenerative disease. However, the specific contribution of each cell type to the neuroinflammatory axis in vivo remains unclear with species-specific differences in key signaling pathways further complicating the challenge.

Over recent years, neuroinflammation has been increasingly implicated in the progression of various neurodegenerative disorders such as Alzheimer's (Deczkowska, A. et al. Cell 173, 1073-1081 (2018); Keren-Shaul, H. et al. Cell 169 (2017)), Parkinson's (Lecours, C. et al. Front Cell Neurosci 12 (2018) ; Olanow, C. W. et al. Brain 142, 1690-1700 (2019)), Amyotrophic Lateral Sclerosis (ALS) (Geloso, M. C. et al. Front Aging Neurosci 9 (2017)) as well as in aging. Microglia are thought to be a key player in triggering an inflammatory state in the brain which can precipitate or exacerbate disease pathology. Other glial cells such as astrocytes interact with microglia and may further contribute to aberrant inflammation and cause neurotoxicity (Liddelow, S. A. et al. Nature 541, 481-487 (2017); Rostalski, H. et al. Front Neurosci 13 (2019)). However, parsing out cellular crosstalk in vivo and understanding species specific differences in the neuroinflammatory response (Smith, et al., Trends Neurosci 37, 125-135 (2014)) have been major challenges in the field. Human pluripotent stem cell (hPSC) technology has the potential to overcome both of those challenges and to present a fully human, defined and scalable platform to study neuroinflammation. An essential requirement for such an hPSC-based model are differentiation strategies that can reproducibly generate pure populations of microglia, astrocytes, and neurons in a synchronized, efficient, and timely manner. Protocols based on dual-SMAD inhibition (Chambers, S. M. et al. Nat Biotechnol 27, 275-280 (2009); Qi, Y. et al. Nat Biotechnol 35, 154-163 (2017)) allow for the efficient production of highly pure neural precursors and postmitotic neurons from hPSCs. Likewise, it was recently reported a strategy to rapidly derive pure populations of astrocytes from hPSCs (Tchieu, J. et al. Nat Biotechnol 37, 267-275 (2019)). In contrast, several protocols have been published for generating microglial-like cells from hPSCs (Muffat, J. et al. Nat Med 22, 1358-1367 (2016); Abud, E. M. et al. Neuron 94, 278-293.e279 (2017); Douvaras, P. et al. Stem Cell Reports 8, 1516-1524 (2017); Haenseler, W. et al. Stem Cell Reports 8, 1727-1742 (2017); Takata, K. et al. Immunity 47, 183-198.e186 (2017); Pandya, H. et al. Nat Neurosci 20, 753-759 (2017); Brownjohn, P. W. et al. Stem Cell Reports 10, 1294-1307 (2018)); but those approaches commonly rely on embryoid body formation and are poorly defined with respect to ontogeny (Muffat, J. et al. Nat Med 22, 1358-1367 (2016); Haenseler, W. et al. Stem Cell Reports 8, 1727-1742 (2017)), require manipulations such as hypoxia for patterning (Abud, E. M. et al. Neuron 94, 278-293.e279 (2017)), or necessitate cell-sorting for purity (Douvaras, P. et al. Stem Cell Reports 8, 1516-1524 (2017)). Importantly, none of the microglial differentiation protocols demonstrate explicit patterning towards primitive hematopoiesis as defined by the induction of KDR+CD235A+ hemangioblasts (Sturgeon, et al., Nat Biotechnol 32, 554-561 (2014)). To recapitulate those early developmental steps in vitro is important as microglia are unique in that their lineage entirely traces back to primitive hematopoiesis Ginhoux, F. et al. Science 330, 841-845 (2010)).

To construct a fully human platform to study neuroinflammation, a novel approach to derive microglia from human pluripotent stem cells (hPSCs) that explicitly recapitulates microglial ontogeny was identified and validated by single cell RNA-sequencing and stage-specific mapping onto datasets of developing mouse microglia. Using these cells, the first defined and completely human tri-culture system containing pure populations of hPSC-derived microglia, astrocytes, and neurons to dissect cellular crosstalk along the neuroinflammatory axis in vitro was built The tri-culture system was generated to model inflammation in Alzheimer's Disease using isogenic hPSCs with the APPSWE^{+/+} mutation. The presently disclosed data revealed that production of complement C3, a protein that is increased under inflammatory conditions and implicated in synaptic loss, was potentiated under tri-culture conditions, and was further enhanced in APPSWE+/+tri-cultures. Using cell type-specific ablation studies, it was found that C3 potentiation is due to the presence of a neuroinflammatory loop in which microglia were the key initiators that activated astrocytes to produce excess C3. The presently disclosed study defined the major cellular players contributing to increased C3 in AD and presented a broadly applicable platform to study neuroinflammation in human disease.

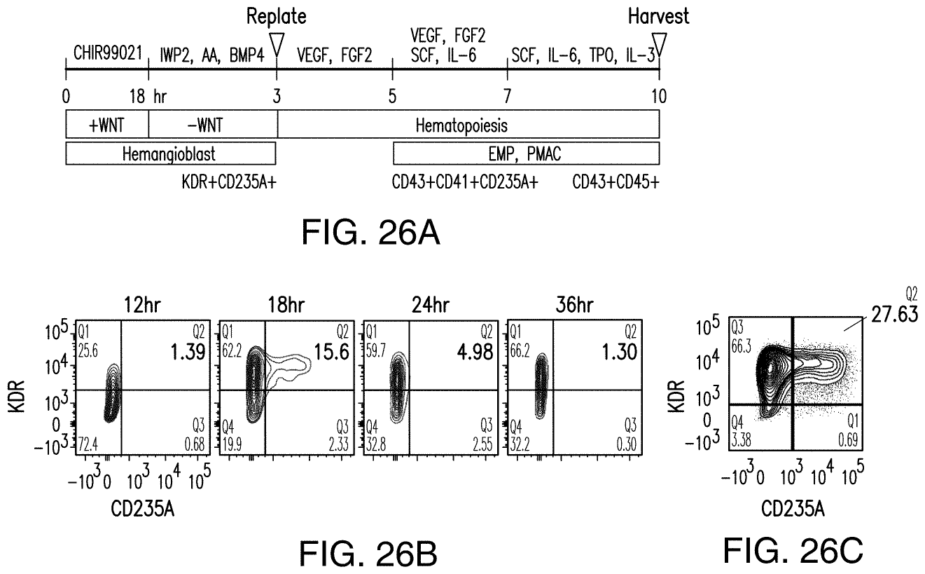

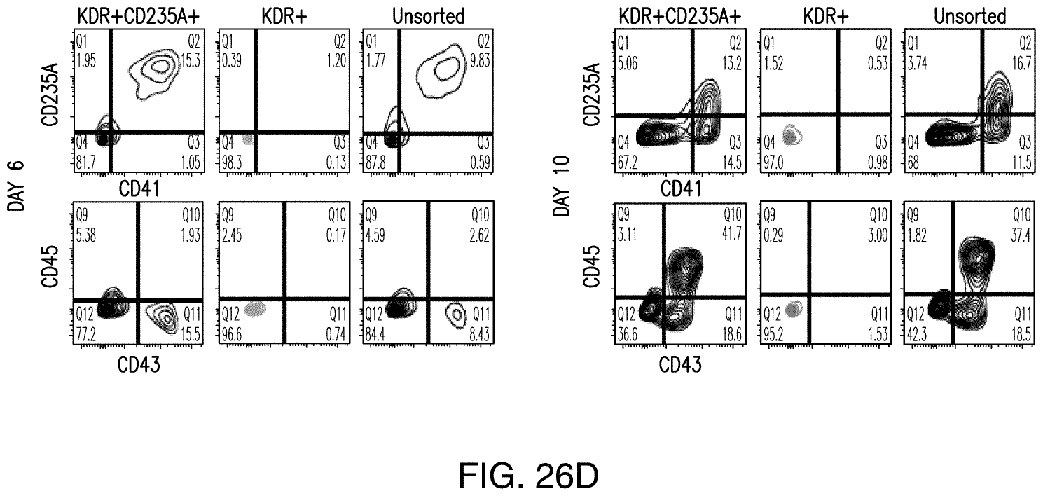

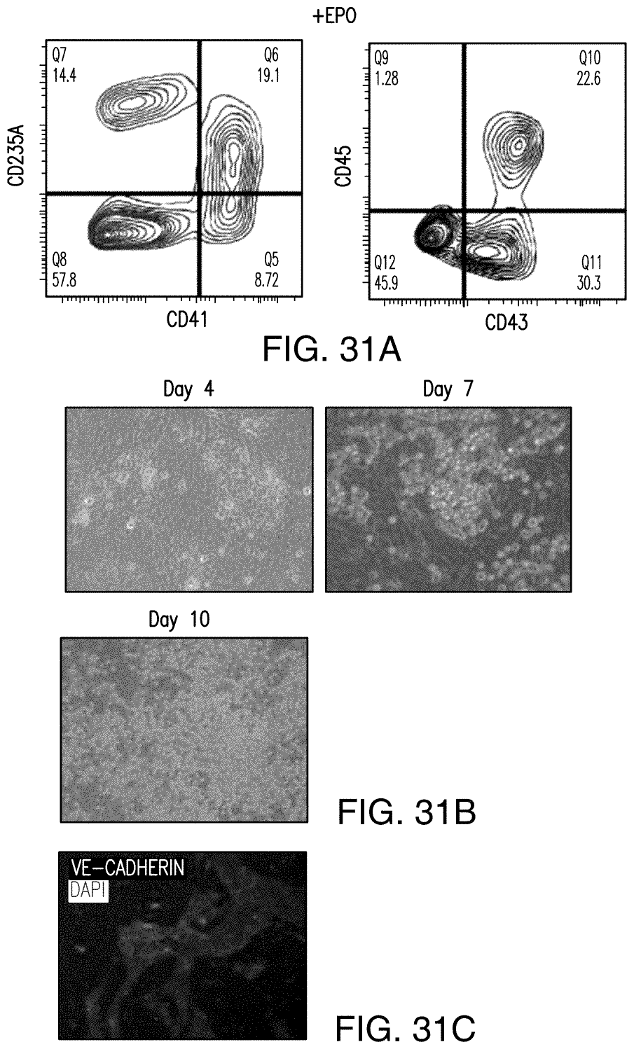

The presently disclosed strategy was designed to differentiate hPSCs into microglial precursors (FIG. 26A). First, cells were treated with the GSK3b inhibitor CHIR99021 (Lindsley et al., Development (2006); 133, 3787-3796) and patterned towards primitive streak mesoderm by activating WNT signaling . Then, WNT inhibition was induced by the porcupine inhibitor IWP2 and Nodal signaling was concurrently activated, mimicked by exposure to Activin A. Those conditions biased the generation of KDR+CD235A+ primitive hematopoietic hemangioblasts versus KDR+CD235A− definitive hematopoietic precursors following the paradigm proposed in Sturgeon et al. (Sturgeon et al., Nature Biotechnol. (2014); 32, 554-561). It was found that WNT inhibition must have occurred within a very narrow developmental window, limited to 18 hours post WNT activation, to efficiently generate a KDR+CD235A+ population (FIG. 26B). After optimizing conditions for cell density and small molecule exposure (see material and methods), WNT activation for 18 hour followed by WNT inhibition and Nodal activation for 2 days yields ˜30% KDR⁺CD235A⁺ cells by Day 3 of differentiation (FIG. 26C). Next, it was determined whether KDR⁺CD235A⁺ hemangioblasts vs. the KDR+CD235A− definitive precursors generated hematopoietic cells under those culture conditions. The sorted KDR⁺CD235A⁺ hemangioblast population was observed that they produced CD41⁺CD235A⁺CD43⁺ hematopoietic cells within 3 days after re-plating (Day 6 of hPSC differentiation) in the presence of minimal hematopoietic cytokines (FIG. 26D). In contrast, the KDR⁺CD235A⁻ hemangioblast population did not produce hematopoietic cells (FIG. 26D). The presently disclosed data demonstrated that definitive precursors generated hematopoietic cells only at later stages of differentiations, under hypoxic conditions, or in the presence of additional hematopoietic cytokines. By Day 7 after re-plating, the KDR⁺CD235A⁺ fraction yielded 41% CD45⁺ cells, the population that later produced microglia. Other cell populations included uncommitted CD41⁺CD235A⁺CD43⁺ erythromyeloid progenitors (EMPs), CD41⁺ megakaryocytes, and CD235A⁺ erythrocytes (only when treated with erythropoietin) (FIG. 31A). EMPs, megakaryocytes, and erythrocytes were all lineages produced during primitive hematopoiesis (Palis et al., FEBS Lett. (2016); 590, 3965-3974) (FIG. 26D). The definitive KDR⁺CD235A⁻ population still did not produce hematopoietic cells, which demonstrated all hematopoietic cells produced by Day 10 of differentiation, were derived from KDR⁺CD235A⁺ hemangioblasts. Interestingly, unsorted samples containing both the KDR⁺CD235A⁻ and KDR⁺CD235A⁺ populations produced hematopoietic cells with nearly the same efficiency as the sorted KDR⁺CD235A⁺ population. These results suggested that KDR⁺CD235A⁺ cells do not need to be purified for the robust production of hematopoietic cells (FIG. 26D). This finding streamlines the differentiation process by eliminating the cell sorting step and instead allowing the simple replating of the mixed population at Day 3 in the presence of hematopoietic cytokines. Almost 60% of the cells at Day 10 of differentiation are of hematopoietic identity forming floating colonies in semi-suspension above a VE-CADHERIN⁺ hemogenic endothelium (Raffi et al., Blood (2013); 121, 770-780) (FIGS. 31B and 31C).

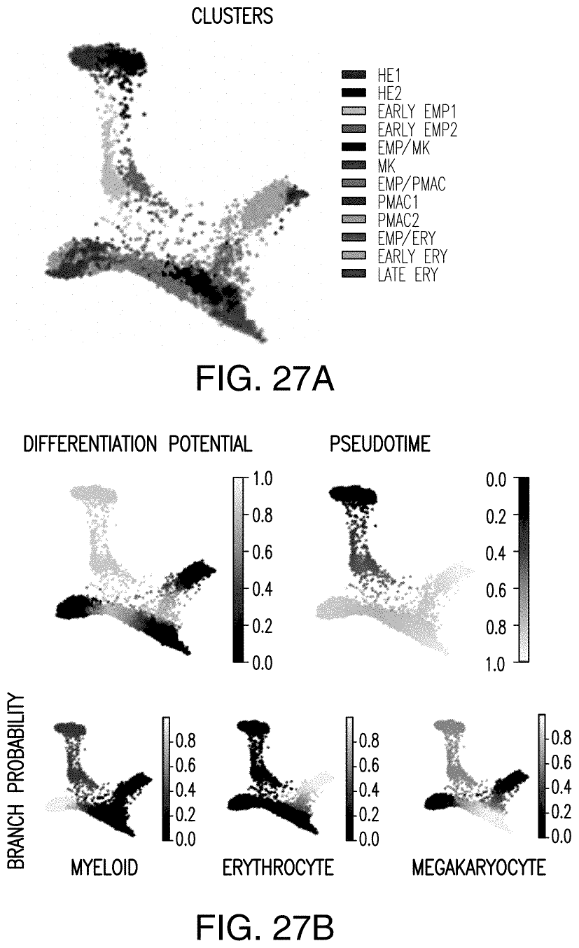

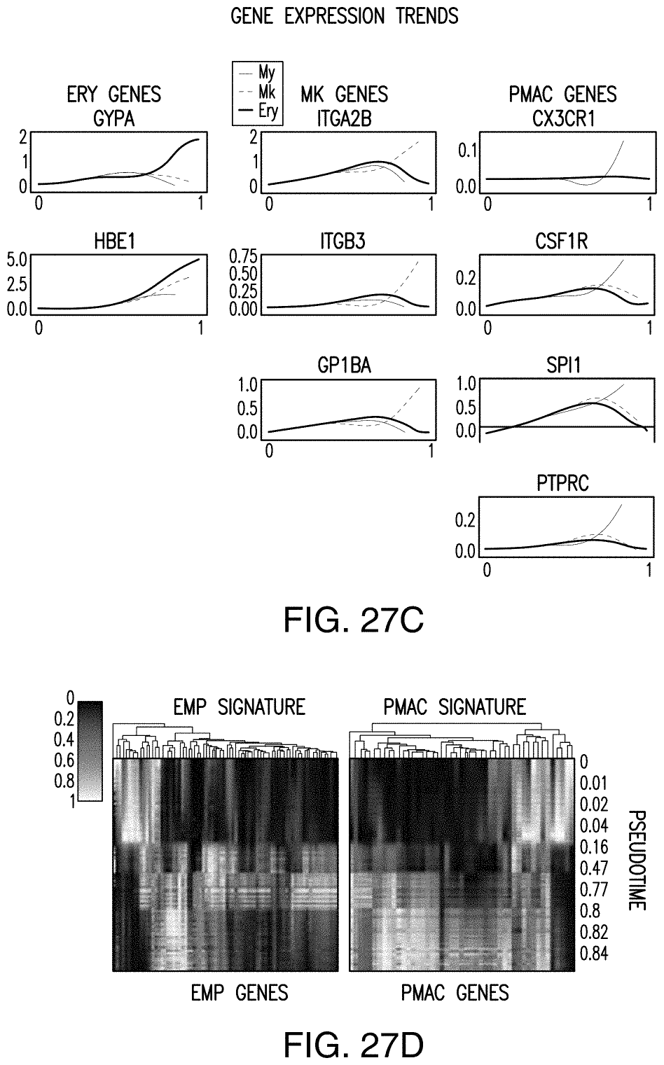

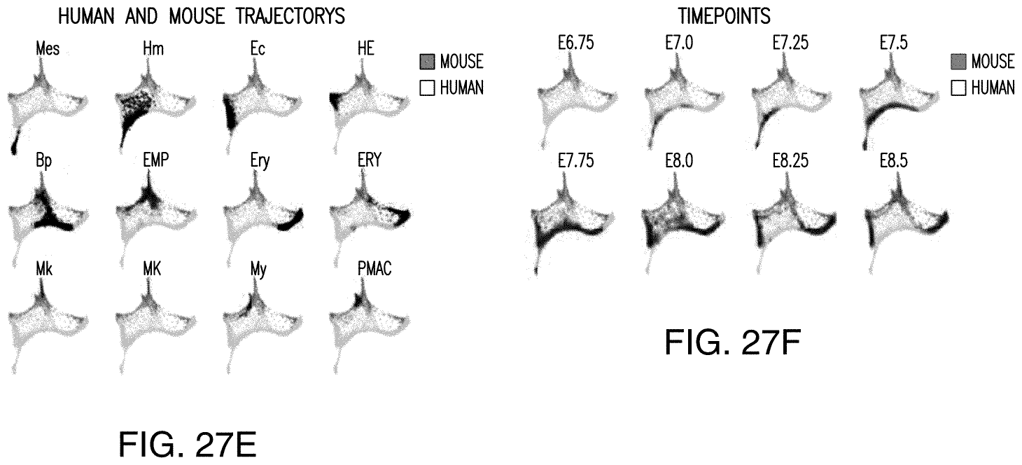

Next, single cell RNA-sequencing experiments were conducted at Day 6 and Day 10 of differentiation to fully characterize the heterogeneity of the cells and identify the developmental trajectory of the transitioning cellular states given the paucity of data on human primitive hematopoiesis in current literature. The composition and trajectory of the differentiation were compared with those derived from in vivo mouse development to verify whether the presently disclosed methods indeed produced primitive hematopoiesis in a dish. Pooled Day 6 and Day 10 single cells RNA-sequencing data were separated into discrete clusters along a defined trajectory after diffusion mapping analysis (FIG. 27A). The more mature hematopoietic populations of erythrocytes (ERY), megakaryocytes (MK), and macrophage precursors (PMAC) emerged from three distinct arms stemming from a common EMP population, mimicking the trajectory of primitive hematopoiesis. The differentiation potential of the cells were calculated over pseudotime along the trajectory using the Palantir algorithm (Setty et l., Nat. Biotechnol. (2019); 57, 451-460) and the results showed that the highest differentiation potential fell within the hemogenic endothelial clusters and the EMP like cells at the center of the map, and the lowest fell at the more mature hematopoietic clusters at the ends of the three arms (FIG. 27B). The expression of key genes was also calculated over pseudotime along the trajectory of each arm (FIG. 27C). The erythrocyte arm expressed the signature genes of embryonic hemoglobin (HBE1) and GYPA, and the megakaryocyte arm expressed key megakaryocytic genes of ITGA2B, ITGB3, and GP1BA, and the macrophage precursor arm expressed CSF1R, PTPRC, and CX3CR1 (Kierdorf et al., Nat. Neurosci. (2013); 16, 273-280). The trajectory was isolated along only the macrophage precursor arm was isolated and compared its gene expression to the gene signatures of EMPs and pre-macrophages (PMACS) derived from in vivo profiling of mouse microglial development (Mass et al., Science (2016); 353) (FIG. 27D). At pseudotimes of 0.44-0.8 corresponding to the early EMP and EMP/PMAC clusters, the presently disclosed data enriched for the mouse EMP signature, and at pseudotimes of 0.84 and above corresponding to the mature PMAC1/2 clusters, the data enriched for the mouse pMAC signature (Mass et al. Science (2016); 353). However, of the 90 and 51 genes present in the mouse EMP and pMAC signatures respectively, 81 and 49 genes were present in the presently disclosed scRNAseq dataset, and only a subset of these genes were separately expressed in the human EMP or pMAC clusters with some genes expressed in both (FIG. 27D), likely representing human to mouse differences. These data were also mapped onto whole mouse gastrulation single cell RNA-sequencing data (Pijuan-Sala et al., Nature (2019); 566, 490-495) and noted the clusters of hematopoietic cells matched closely to the mouse hematopoietic cells. Specifically, the PMAC cluster mapped near the mouse gastrulation myeloid cluster (FIG. 27E). When compared with data derived from multiple timepoints of the developing mouse gastrula, the presently disclosed data contained populations that were the most similar to those that emerged at E8.5, 1 day before microglial precursor cells initially seeded the mouse brain (FIG. 27F). Taken together, these data demonstrate that the presence of EMPs and EMP-derived pMACs in the presently disclosed cultures closely match mouse myeloid cells at the time of early microglial development, indicating that the presently disclosed human in vitro culture system follows the postulated developmental roadmap for microglial development.

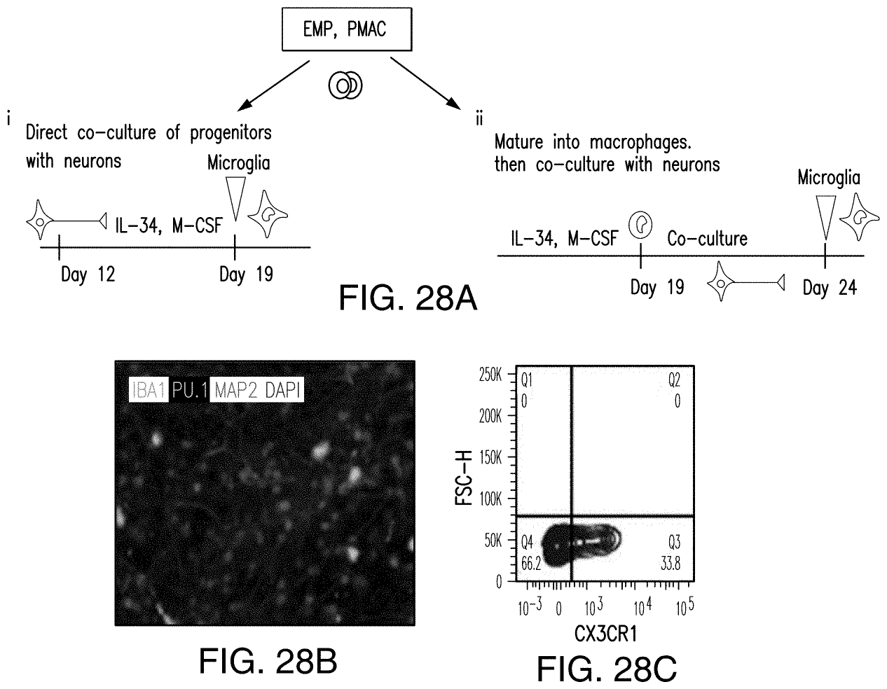

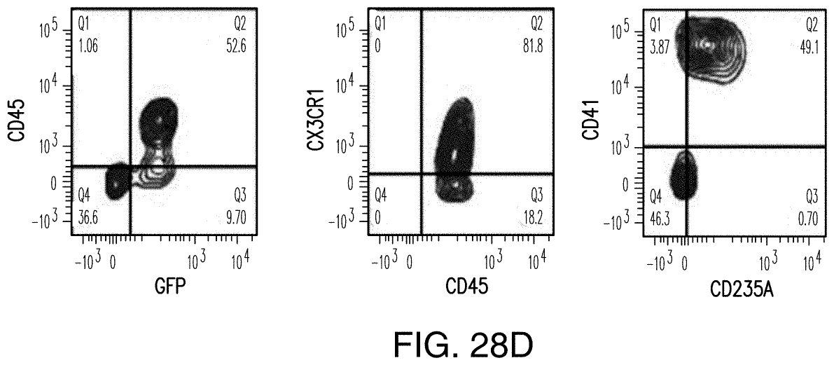

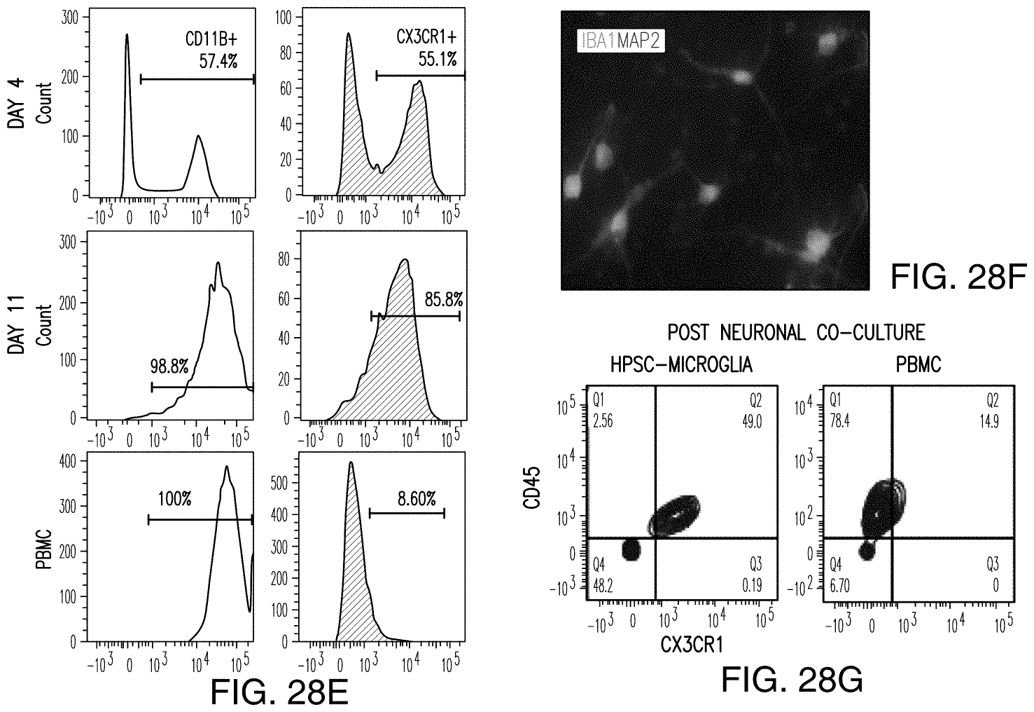

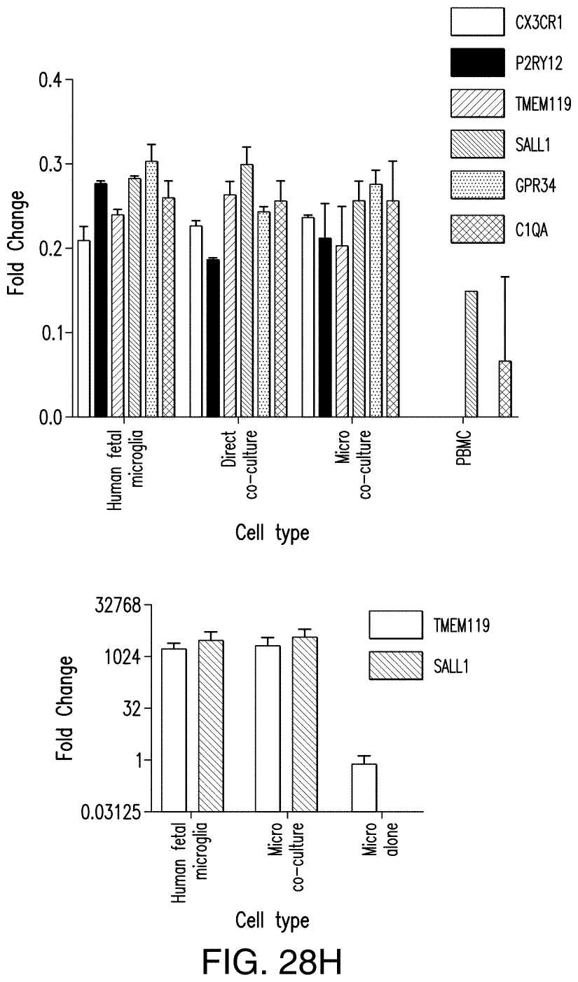

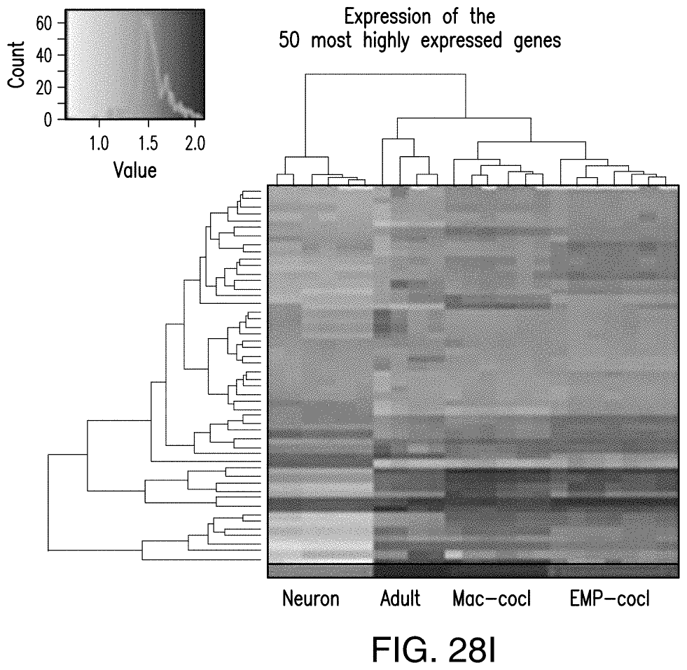

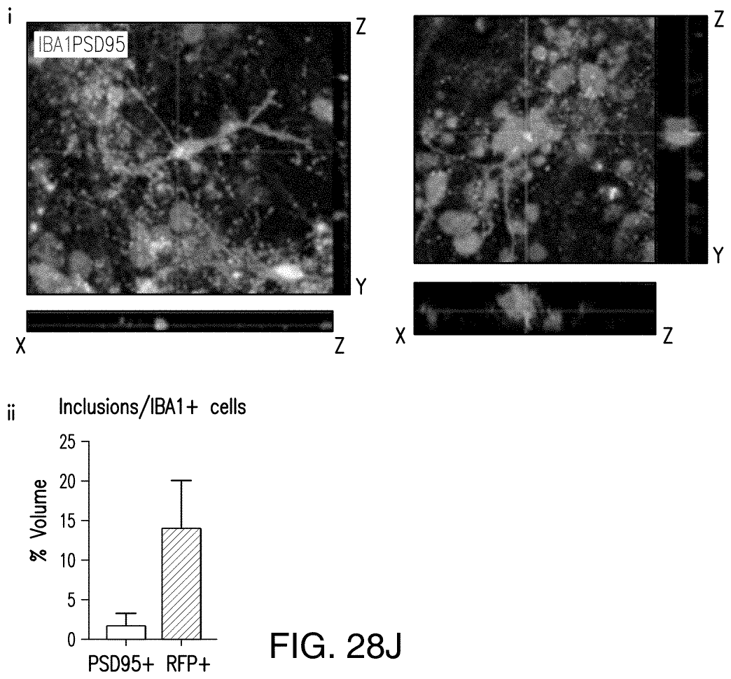

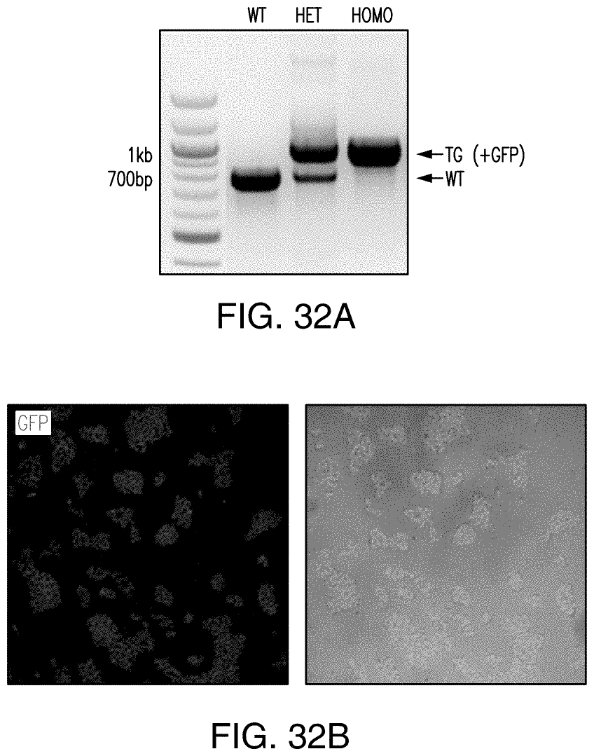

Two separate methods that can functionally mature either EMPs or pMACs into microglia were established in the presently disclosed (FIG. 28A). The first method mimicked the in vivo developmental trajectory during which microglial precursors seeded the brain and developed into microglia within the neural environment (Pijuan-Sala et al., Science 2016; 353). To recapitulate this paradigm, cells in suspension were harvested at Day 10 and directly co-cultured with Day 30 hPSC-derived cortical neurons in the presence of IL-34 and M-CSF, cytokines that are important for microglial survival and maturation (Wang et al., Nat. Immunol. (2012);13, 753-760) (FIG. 28A, panel i). 50,000 cells in suspension were plated per 300,000 cortical neurons, yielding ˜16% hematopoietic cells per culture at the time of plating. Remarkably, adherent, ramified, and microglial-like cells emerged expressing IBA1 and PU.1 within 4 days of co-culture (FIG. 28B). These cells also expressed CX3CR1⁺ and made up more than 30% of the co-cultures indicating ongoing cell proliferation in parallel to the differentiation towards microglial lineage (FIG. 28C). To address whether other primitive hematopoietic lineages emerge in these co-cultures, GFP⁺ hematopoietic cells from a GPI⁻H2B⁻GFP hPSC line were generated (FIGS. 32A-32B) and co-cultured them with hPSC-derived cortical neurons. At Day 6 of co-culture, the majority of GFP⁺ cells were CD45+, which indicated that they differentiated along a microglial rather than megakaryocytic or erythrocytic trajectory. 82% of these CD45⁺ cells expressed CX3CR1, which suggests that most CD45⁺ cells had transitioned into a microglial fate (FIG. 28D). Approximately 10% of GFP⁺ cells were not CD45⁺. Half of those cells were immature CD41⁺CD235A⁺ EMPs, whereas the other half were negative for these markers, possibly indicating an even earlier hematopoietic lineage. These data demonstrate that co-culturing Day 10 EMPs and PMACs with cortical neurons yields a population of microglial cells within 4 days, though small populations of uncommitted hematopoietic cells may persist.

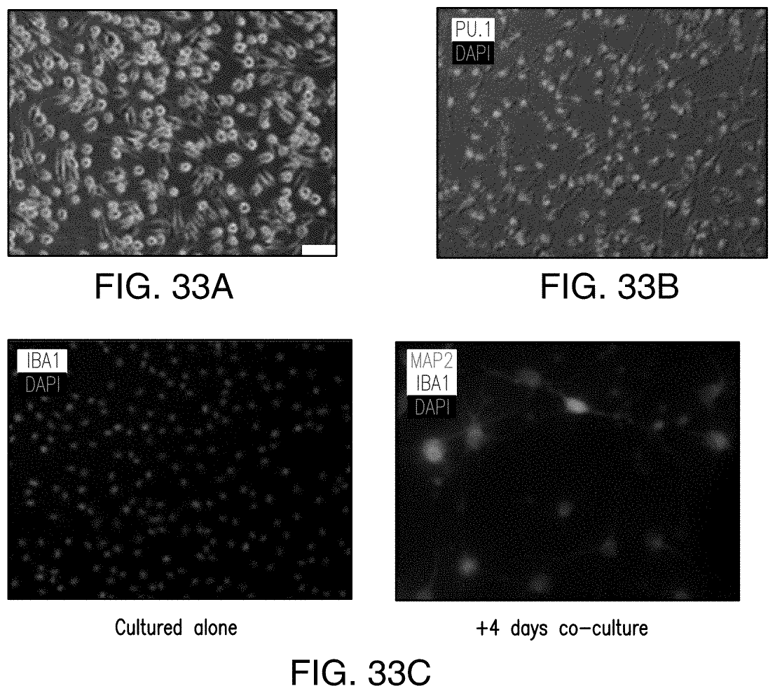

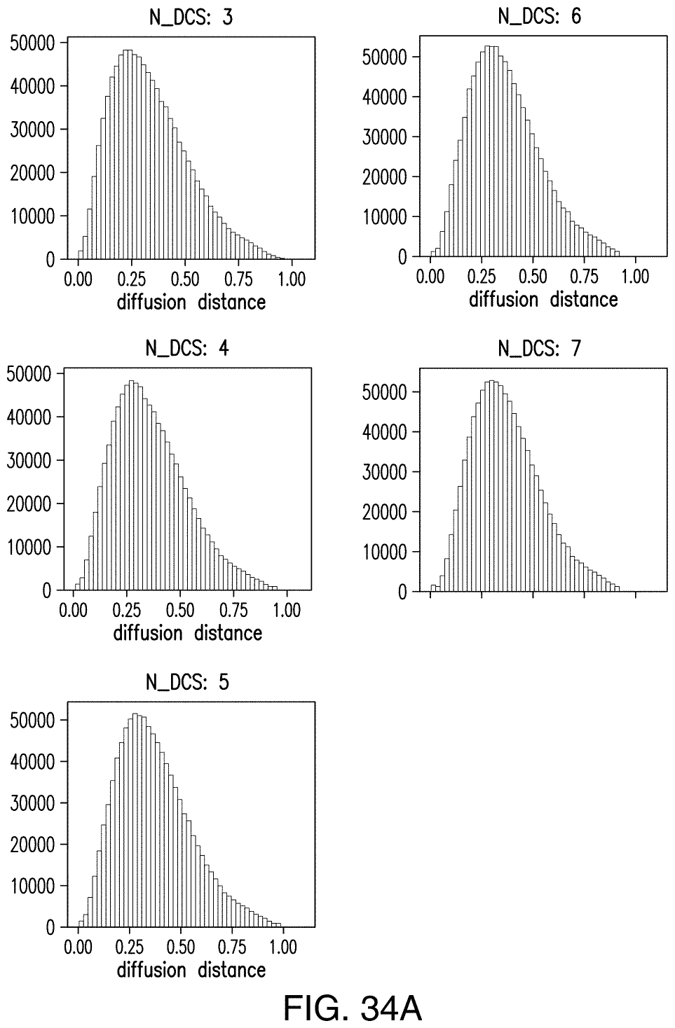

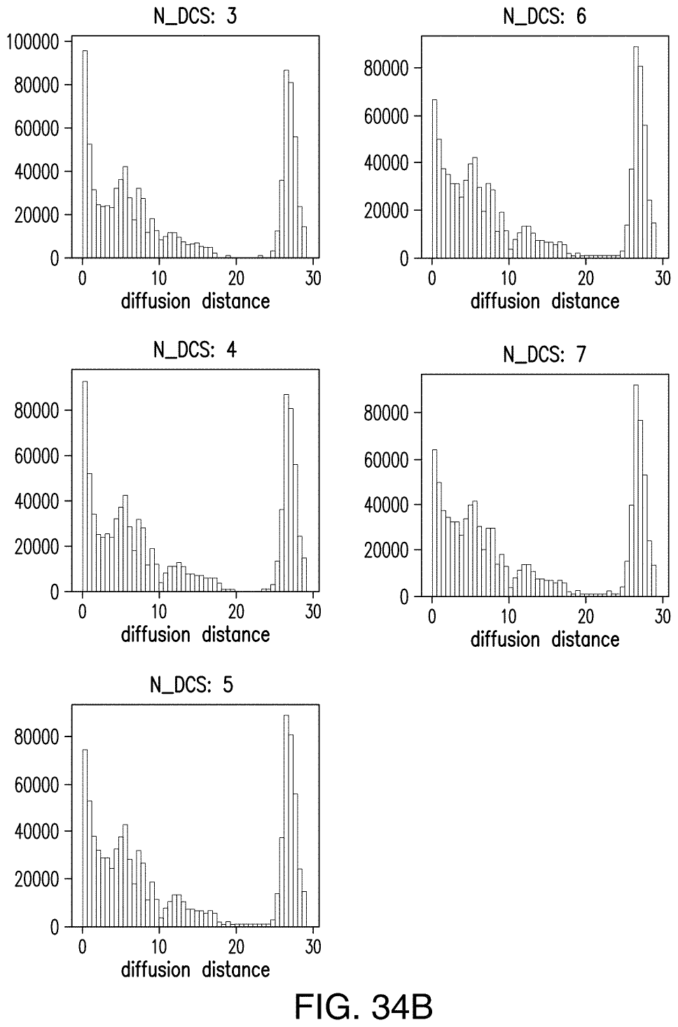

A second strategy of maturing microglia from the progenitor stage was developed to derive a completely pure and synchronized population of microglia, a (FIG. 28A, panel ii). The bulk population of hematopoietic cells were pooled in suspension at Day 10, followed by exposure to either serum containing medium (RPMI+10% serum with the addition of IL-34 and M-CSF) or by using defined, serum-free conditions (IMDM/F12 with the addition of IL-34 and M-CSF) for 7-11 days. At 4 days of culture, half of the cells had transitioned to the primitive macrophage stage, with 50-60% expressing CD11B (mature macrophage/microglial marker) and CX3CR1 (restricted to tissue-resident macrophages such as microglia). By 11 days of culture, close to 99% of the cells expressed CD11B and over 85% expressed CX3CR1 (FIG. 28E). At this stage, all cells were adherent, displayed an elongated morphology, and were PU.1⁺ (FIGS. 33A and 33B). In contrast, primary human peripheral blood mononuclear cells (PBMCs) matured in parallel under the same culture conditions and expressed CD11B but largely lacked CX3CR1 expression (FIG. 28E). The resulting pure population of primitive macrophages were co-cultured with hPSC-derived cortical neurons to fully transition these cells to microglial fate. After 4 days of co-culture, the microglial cells displayed ramifications and upregulated IBA1 (FIG. 28F and FIG. 33C). When compared to matured PBMCs that were co-cultured with neurons, the microglial-like cells had lower levels of CD45 and maintained CX3CR1 expression, whereas the PBMC-derived cells expressed CD45 in the absence of CX3CR1 (Greter et al., Front Immunol. (2015); 6) (FIG. 28G). To determine transcriptional identity, hPSC-derived microglial cells were sorted out using CD45⁺CX3CR1⁺, 2 weeks after initiation of co-culture. It was observed expression of signature microglial genes at levels similar to human fetal microglia (Butovsky et al., Nat. Neurosci. (2014);17, 131-143; Bennett et al., Proc. Natl. Acad. Sci. U.S.A. (2016); 113, E1738-1746) (FIG. 28H). Interestingly, TMEM119 and SALL1 were markedly increased upon neuronal co-culture (Gosselin et al., Science (2017); 356) (FIG. 28H). Single-cell RNA sequencing of the co-cultured hPSC-derived microglial cells revealed that the cells represented a homogenous cell population devoid of undifferentiated precursors (FIGS. 34A-34B). Pairwise distances calculated between cells in the microglial sample fall in a clean unimodal distribution, indicating little variance between cells (FIG. 34A). In contrast, pairwise distances calculated between cells at day 10, prior to co-culture, showed multiple peaks in the distribution pointing to the heterogenous nature of the cells (FIG. 34B). Next, it was determined whether either of the two derivation methods yielded cells transcriptionally more similar to primary human microglia. After 14 days of co-culture with cortical neurons, the microglial cells were sorted out using CD45⁺CX3CR1⁺ and their gene expression were compared to primary adult human microglia via bulk RNA sequencing. Following unsupervised hierarchical clustering, both methods yielded microglial cells which cluster to primary human microglia obtained from postmortem cortical brain tissue (frontal and temporal, aged 60-77 years old) (FIG. 28I). However, neither method fell exactly in the same sub-branch as the primary human microglia, perhaps due to the age of the cells (embryonic vs. aged) and/or as a result of in vitro culture.

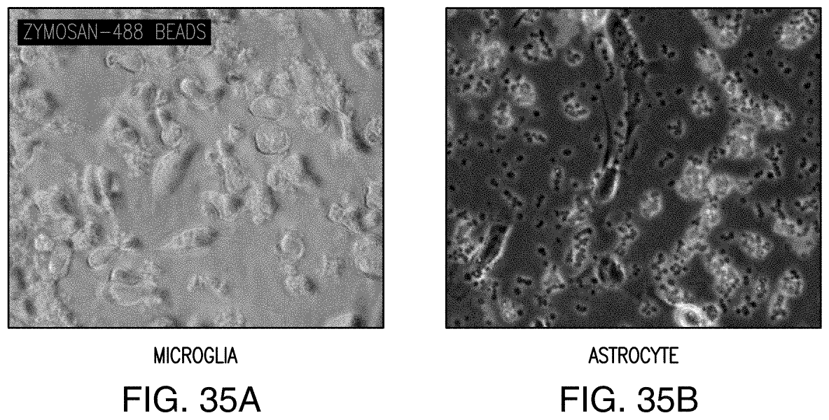

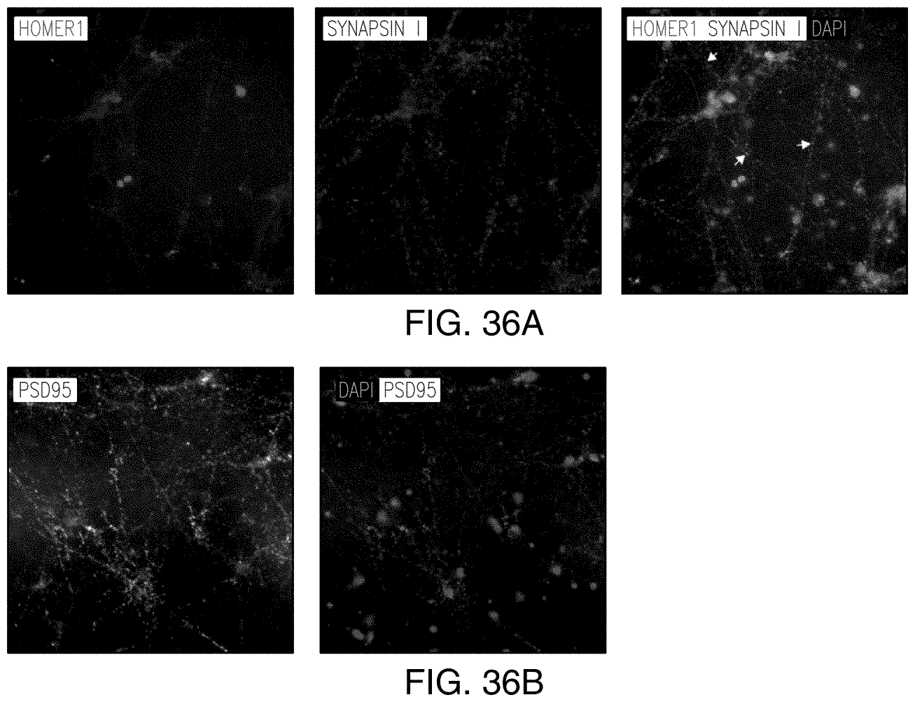

hPSC-derived microglial cells shared functional similarities with in vivo microglia. It was observed that hPSC-derived microglial cells in co-culture with neurons survey their environment, retracting and extending their processes to sample the surrounding neurons, akin to homeostatic microglia in vivo were observed (Nimmerjahn et al., Science (2005); 308, 1314-1318). When challenged with yeast-antigen zymosan, the cells were able to perform more efficient phagocytosis (Wake et al., Neuron Glia Biol (2011); 7, 1-3) compared to an astrocyte control (FIGS. 35A-35B). Finally, another role of microglial cells is to prune synapse in the developing brain (Stevens et al., Cell (2007);131, 1164-1178). When co-cultured with mature hPSC-derived neurons that formed synapses (D70 and older (FIGS. 36A-36B)), microglial cells showed inclusions containing synaptic material upon confocal imaging (FIG. 28J, panel i). While there were inclusions that contained general neuronal material, tagged with RFP, the number of inclusions that specifically composed of synaptic materials was 1-2% (FIG. 28J, panel ii). This number matched the basal level of synaptic uptake reported for primary microglial cells during homeostasis (Schafer et al., Neuron (2012); 74, 691-705).

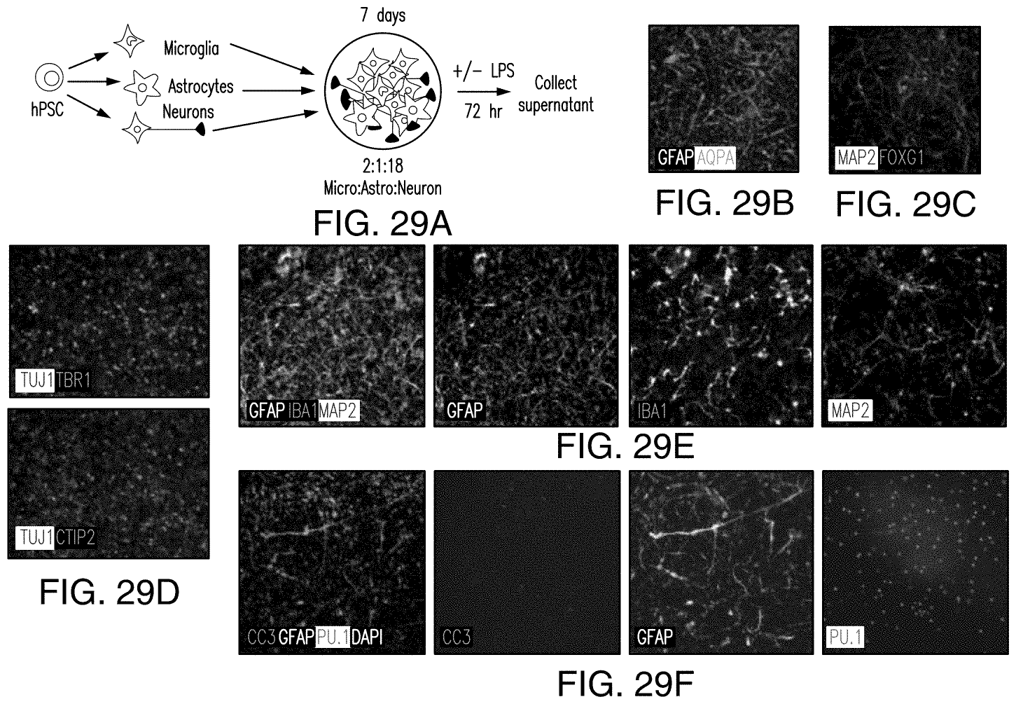

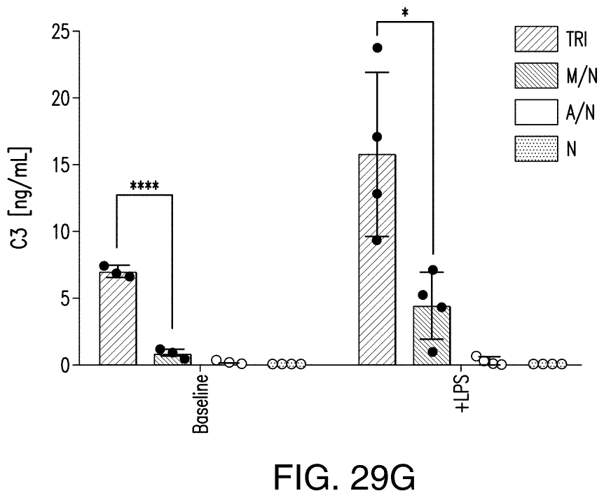

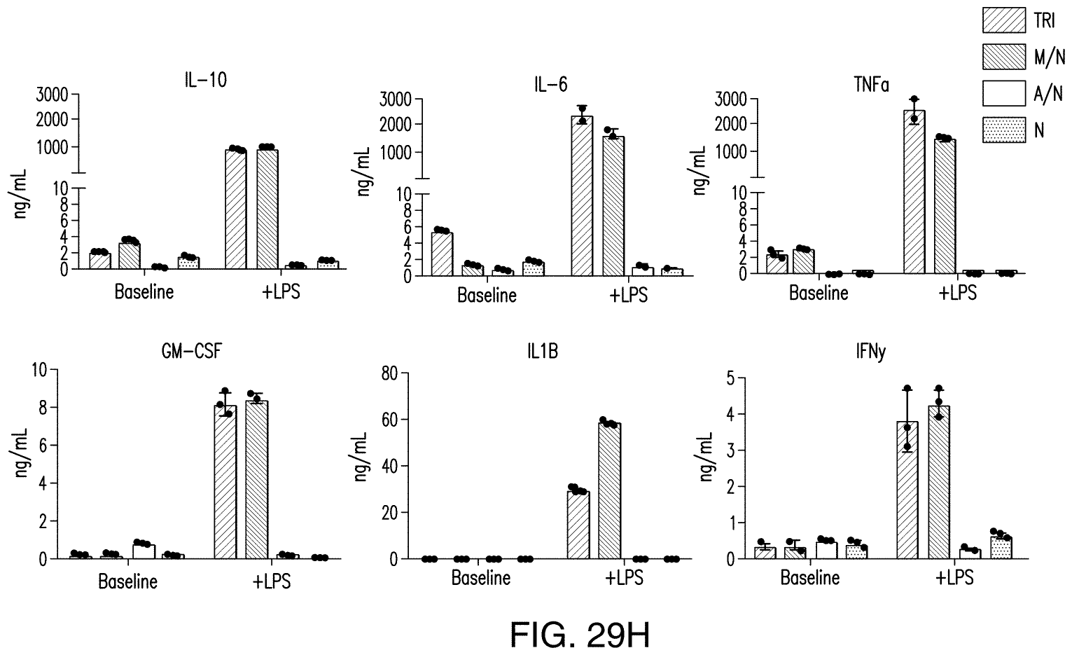

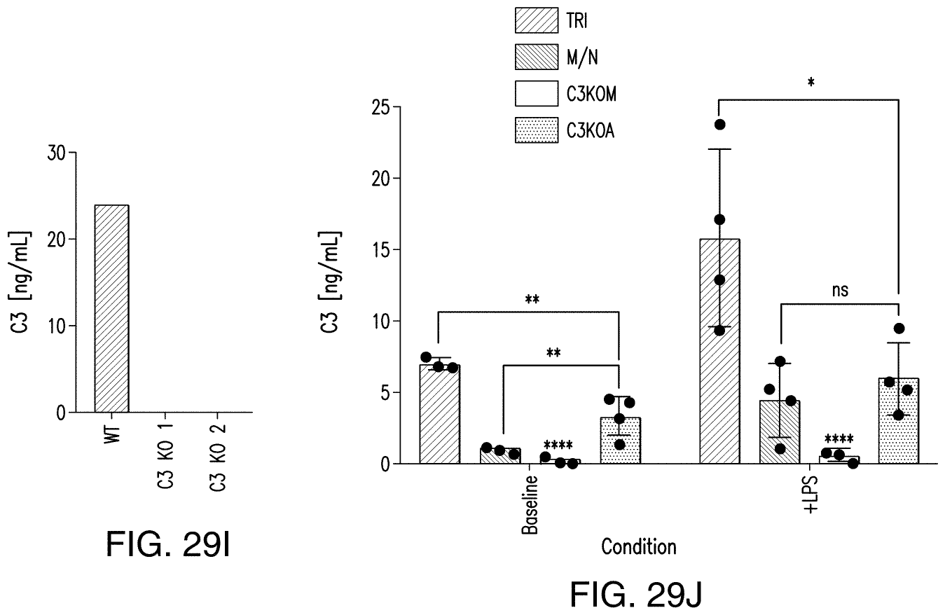

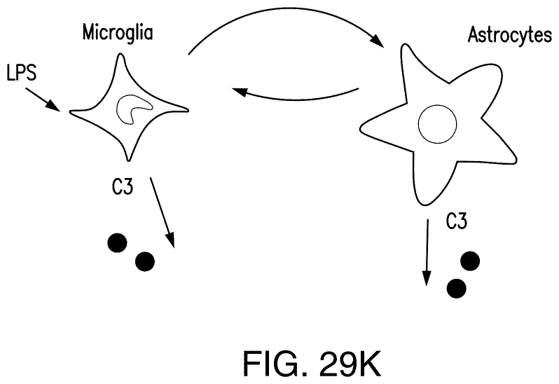

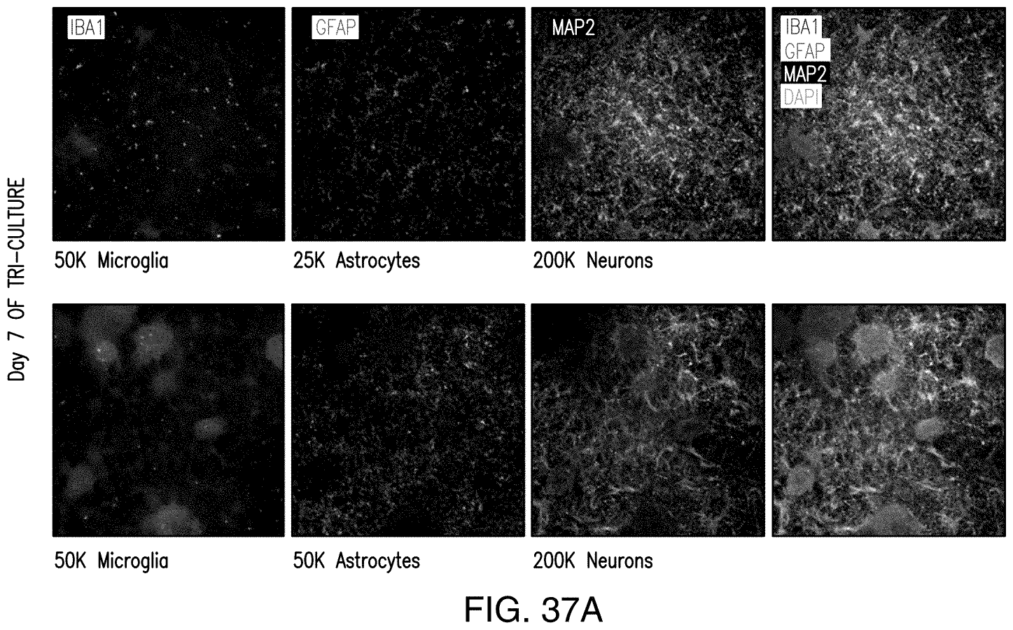

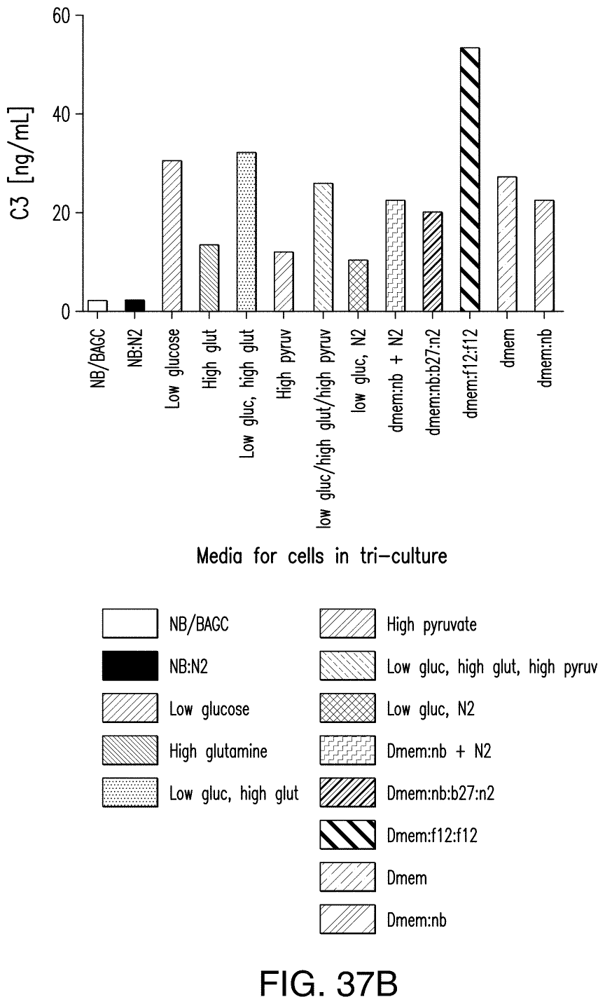

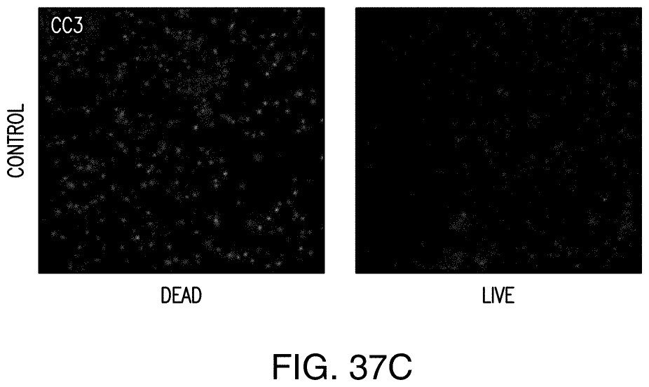

The ability to generate nearly pure microglia within 21 days of differentiation with properties that match their primary counterpart was set for the stage to build a functional, fully hPSC-derived tri-culture platform composed of human microglia combined with similarly pure populations of human astrocytes (Tchieu et al., Nat Biotechnol (2019); 37, 267-275) and cortical neurons (Chambers et al., Nat Biotechnol (2009); 27, 275-280; Qi et al., Nat. Biotechnol. (2017); 35, 154-163) (FIG. 29A). The hPSC-derived astrocytes were generated as described recently (Tchieu et al., Nat Biotechnol (2019); 37, 267-275) and all expressed GFAP+ with a subset expressing AQP4+ (FIG. 29B). Likewise, the presently disclosed example generated pure hPSC-derived neurons were of cortical neuron identity expressing the cortical layer markers of TBR1 and CTIP2 and the telencephalic marker FOXG1 were generated (FIGS. 29C and 29D). For establishing a tri-culture platform, the optimal ratio of each cell type were identified at initial plating at 2:1:8, with 50,000 microglia:25,000 astrocytes: 200,000 neurons per cm². Those conditions allowed for robust microglial cell attachment and survival in the presence of astrocytes, as increased astrocyte numbers interfered with the attachment of microglial cells (FIG. 37A). Base media culture conditions in the presence of IL-34 and M-CSF were further optimized to reduce production of baseline inflammatory cytokines focusing on complement C3 production (FIG. 37B). The NB/BAGC (see methods) condition resulted in very low baseline C3 induction with excellent neuronal survival and maintenance (Qi et al., Nat. Biotechnol. (2017); 35, 154-163). After one week, tri-cultures showed ramified IBA1+ microglial cells and many GFAP+ astrocyte processes interacting with MAP2⁺ cortical neurons (FIG. 29E). The tri-cultures were largely devoid of any apoptotic and CC3+ cells confirmed excellent survival of all three cell types (FIG. 29F and FIG. 37C).

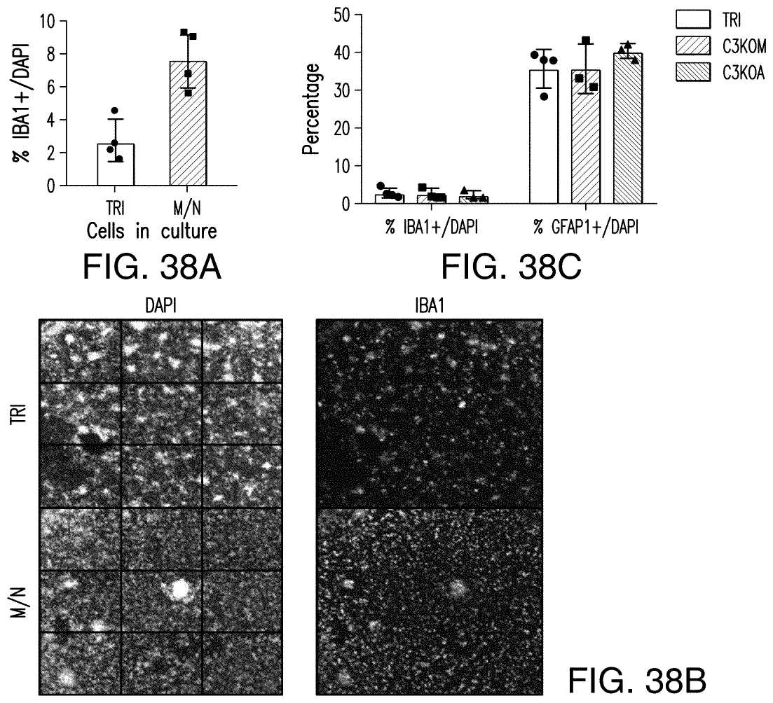

To test whether the triculture system can recapitulate the neuroinflammatory axis between microglia, astrocytes, and neurons, the production of complement C3 as a surrogate marker was measured. First, C3 secretion was assessed by ELISA under various co-culture conditions: neurons only (200,000 cells/cm²), astrocytes and neurons (25,000+200,000 cells/cm²) microglia and neurons (50,000+200,000 cells/cm²), and tri-culture (50,000 microglia/cm²+25,000 astrocytes/cm²+200,000 neurons/cm²). At baseline, C3 was only present in cultures that contained microglia while C3 levels were extremely low in astrocyte/neuron co-cultures and not detected in neuron only cultures. Interestingly, in tri-cultures, the baseline C3 levels were dramatically higher than in microglia/neuron only cultures, suggesting a potentiation of C3 secretion via cellular cross talk in the presence of both microglia and astrocytes (FIG. 29G). C3 secretion was measured after the cultures were stimulated with the inflammatory protein lipopolysaccharide (LPS) to pharmacologically model a neuroinflammatory state (Chen et al., J. Neurosci. (2012); 32, 11706-11715). After LPS treatment, the levels of C3 was increased in all cultures containing microglia, but again greatly potentiated under tri-culture conditions (FIG. 29G). To rule out the possibility that such potentiation could simply reflect an increase in microglial numbers, IBA+ cells by immunofluorescence was quantified using a high-content imaging microscope. Interestingly, IBA1⁺ positive cells were actually decreased in tri-cultures versus microglia/neuron co-cultures ruling out an increase in microglial numbers as the cause of higher C3 levels (FIGS. 38A and 38B). LPS stimulation induced an inflammatory state beyond C3 secretion along with the increased levels of classical inflammatory cytokines by ELISA such as IL-6, TNF, and IL-10 (FIG. 29H).

A C3 KO hPSC line was generated using CRISPR/Cas9 which showed a complete lack of C3 production (FIG. 29I). This line was differentiated into C3 KO astrocytes and C3 KO microglia and tri-cultures were generated that contained C3 KO astrocytes, wildtype microglia, and neurons (C3KOA) or wildtype astrocytes, C3 KO microglia, and neurons (C3KOM). The number of microglia scored by % IBA1/DAPI and the number of astrocytes scored by % GFAP/DAPI were similar across the wildtype tri-culture, C3KOA, and C3KOM cultures (FIG. 38C). At baseline, the C3KOA cultures showed reduced C3 levels as compared with WT tri-cultures but higher levels than a microglia/neuron culture (FIG. 29J). Interestingly, in the C3KOM cultures, C3 levels were very low, indicating that microglia must express C3 in order to effectively induce astrocytes to produce C3 in tri-culture (FIG. 29J). Whether this microglial C3 acts directly on astrocytes or via autocrine stimulation of microglia indirectly remains to be determined. Comparable results are obtained following LPS stimulation. C3KOA cultures stimulated with LPS show lower C3 levels than a WT culture stimulated with LPS but higher levels than a microglia/neuron only culture stimulated with LPS. C3KOM cultures stimulated with LPS show very low levels of C3 (FIG. 29J). These data characterizes cellular crosstalk between microglia and astrocytes in an inflammatory loop present both at baseline and exacerbated upon pharmacological induction of a neuroinflammatory state (FIG. 29K). In this loop, C3-producing microglia are the initiating cell which signal to astrocytes to produce C3. The C3KOA results indicate that astrocytes in turn re-induce microglia to produce more C3.

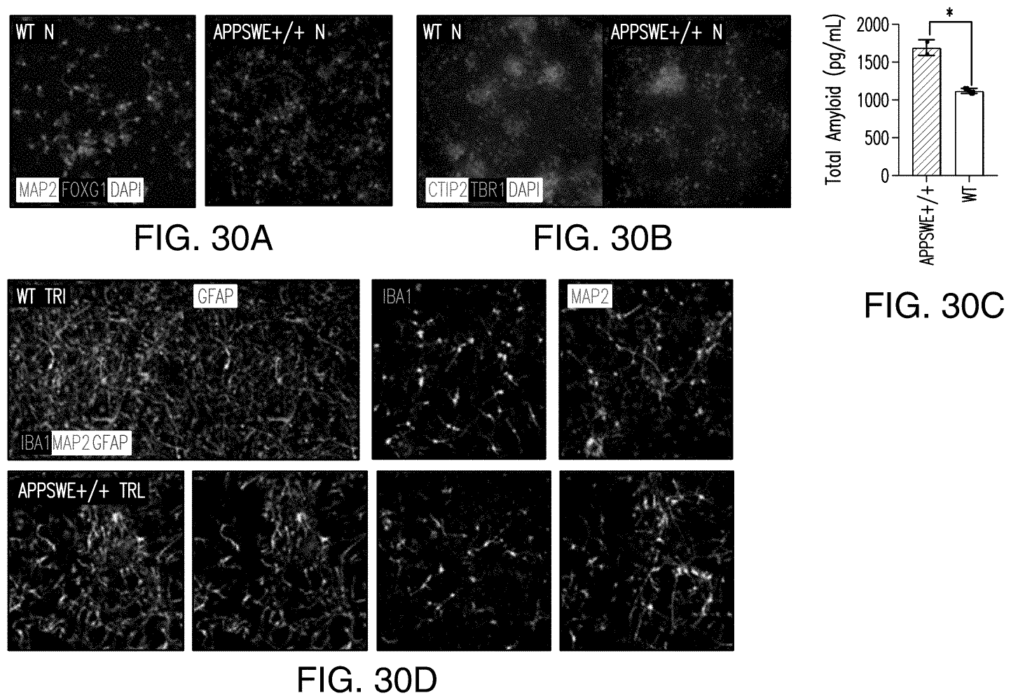

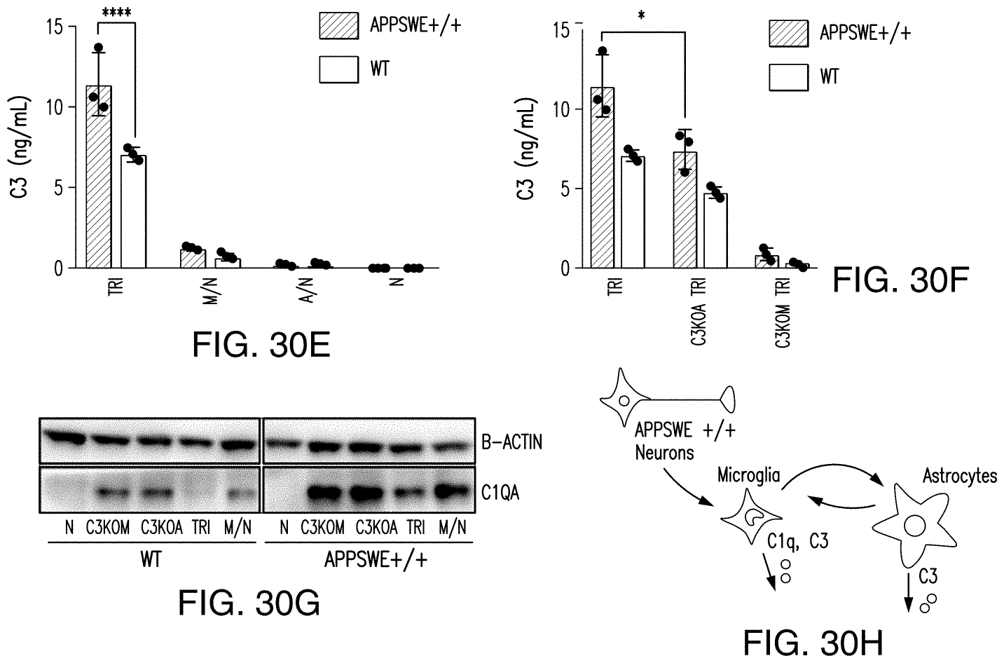

Given the ability of the presently disclosed in vitro tri-culture system to tease apart the cellular components of the neuroinflammatory axis, this platform was applied to model neuroinflammation in a disease state, Alzheimer's Disease. A targeted, isogenic human ESC line containing the APPSWE^{+/+} mutation was used (Paquet et al., Nature 2016; 533, 125-129). Differentiated AD− and isogenic control hPSC-derived neurons were validated by FOXG1 and MAP2 expression as well TBR1 and CTIP2 for cortical identity (FIG. 30A and FIG. 30B). The APPSWE^{+/+} neurons showed increased amyloid-beta production, a hallmark of the APPSWE model of Alzheimer's Disease (Paquet et al., Nature 2016; 533, 125-129) (FIG. 30C). WT differentiated astrocytes (GFAP+) and WT differentiated microglia (IBA1+) in co-culture with matured Day 80 APPSWE^{+/+} neurons or isogenic control neurons were plated to construct tri-cultures (FIG. 30D). At Day 8 of tri-culture, the C3 levels in the tri-cultures containing APPSWE^{+/+} neurons vs. those containing isogenic control neurons were measured by ELISA. Interestingly, C3 levels were higher in the APPSWE^{+/+} tri-cultures when compared to those derived from the isogenic control (FIG. 30E). C3 was not produced at the high levels or increased in APPSWE^{+/+} astrocyte/neuron and APPSWE^{+/+} neuron only cultures vs. isogenic control astrocyte/neuron and neuron only cultures, indicating that microglia must be present in the cultures for robust C3 production.

In order to determine whether the source of the increased C3 in the APPSWE^{+/+} tri-cultures was due to astrocytes or microglia, microglia and astrocytes from C3 KO hPSCs were generated and set up for the tri-cultures containing C3 KO astrocytes, wildtype microglia, and APPSWE^{+/+} neurons or isogenic control neurons (C3KOA), or wildtype astrocytes, C3 KO microglia, and APPSWE^{+/+} neurons or isogenic control neurons (C3KOM). Remarkably, in C3KOA AD tri-cultures, greatly reduced levels of C3 was observed comparable to those of isogenic control tri-cultures (FIG. 30F). However, C3KOA AD tri-cultures still showed increased C3 levels compared to C3KOA isogenic tri-cultures. In C3KOM cultures, the levels of C3 production were low and C3-expressing microglia must be present in order to induce C3 expression in APPSWE^{+/+} tri-cultures (FIG. 30F).

Next, C1Q deposition levels were assessed by western blot. C1Q is a complement protein upstream of C3 which complexes with a cleavage product of complement C3 and also tags synaptic material for clearance (Hong, S. et al. Science (2016); 352, 712-716). Strikingly, there was an increase in C1Q protein found in APPSWE^{+/+} cultures, and C1Q was present only in cultures that contained microglia (FIG. 30G). C1Q has been shown to accumulate in AD in vivo (Hong, S. et al. Science (2016); 352, 712-716; Afagh, Exp. Neurol. (1996); 138, 22-32), and here this phenotype in an in vitro model of AD was recapitulated. it was noted that C1Q levels were lowered in the tri-culture compared to microglia/neuron, C3KOA, and C3KOM cultures in both APPSWE^{+/+} and isogenic control cultures (FIG. 30G). It is suggested that there may be negative feedback between C3 levels and C1Q, as cultures with higher C3 levels (tri-culture) showed lower C1Q as compared to cultures with lower C3 (microglia/neuron, C3KOM, C3KOA). However, the exact mechanism for this inverse relationship remains to be determined in future studies.

Based on the results from the presently disclosed in vitro tri-culture system, a model of the cellular contributions to neuroinflammation in Alzheimer's Disease focused on complement C3 was proposed (FIG. 30H). C3 levels in AD were increased compared to isogenic control tri-cultures and this increase can be interpreted due to astrocytic C3 induced by microglia as well as to microglial C3 re-induced by astrocytes. The results show an inflammatory loop involving APPSWE^{+/+} neurons which triggered microglia to induce reciprocal interactions between both microglia and astrocytes. In addition, the increased deposition of C1Q in AD cultures was detected only in the presence of microglia, and independent of their C3 status.

The presently disclosed tri-culture system enables the dissection of cellular crosstalk by genetic manipulation, and allows to study the mechanisms of increased complement C3 production in tri-culture upon LPS stimulation and in a model of AD. C3 was focused on because recent literature suggests that it is increased during aging (Shi et al., J. Neurosci. (2015); 35, 13029-13042) and in neurodegenerative disorders such as AD (Wu et al., Cell Rep. (2019); 28, 2111-2123; Rasmussen et al., Alzheimer's Dement. (2018); 14, 1589-1601) and is implicated in causing aberrant synaptic pruning (Hong et al., Science (2016); 352, 712-716; Shi et al., Sci. Transl. Med. (2017); 9). However, the technology could be readily adapted to study any other disease-relevant neuroinflammatory target pathways. The identification of the key cellular players contributing to the neuroinflammatory axis in humans should enable the development of directed, cell-type specific therapeutic strategies. In fact, the hPSC-derived tri-culture system could serve as a scalable platform for the screening of therapeutic compounds that specifically target crosstalk between microglia, astrocytes, and neurons in Alzheimer's disease or other neurodegenerative disorders.

### Material & Methods

Derivation of Microglia from hPSCs

hPSCs maintained in Essential 8 media were dissociated by Accutase to obtain a single cell suspension. 60,000 cells/cm2 were plated in E8 medium containing Activin A (R&D 338-AC) (7.5 ng/mL), BMP4 (R&D) (30 ng/mL), CHIR 99021 (Tocris) (3 and ROCK inhibitor (Y-27632) (10 μM) onto Matrigel-coated plates. After 18 hr, medium was changed to Essential 6 medium containing Activin A (10 ng/mL), BMP4 (40 ng/mL), and IWP-2 (Selleck) (2 On Day 2, medium was changed to Essential 6 medium containing Activin A (10 ng/mL), BMP4 (40 ng/mL), IWP-2 (2 μM), and FGF2 (R&D) (20 ng/mL). On Day 3, cultures were dissociated with Accutase and replated at 60,000 cells/cm² in Essential 6 medium containing VEGF (R&D) (15 ng/mL), FGF2 (5 ng/mL), and ROCK inhibitor (Y-27632) (10 μm). On Day 4, the ROCK inhibitor was removed and medium was changed to Essential 6 with VEGF (15 ng/mL) and FGF2 (5 ng/mL). On Day 5 and 6, cultures were fed with Essential 6 medium containing VEGF (15 ng/mL), FGF2 (5ng/mL), SCF (200 ng/mL), and IL-6 (20 ng/mL). On Days 7 and 9, medium was changed to Essential 6 with SCF (100 ng/mL), IL-6 (10 ng/mL), TPO (30 ng/mL), and IL-3 (30 ng/mL). On Day 10, the cells in suspension were collected and either 1) co-cultured with cortical neurons in neurobasal containing B27, L-glutamine, and BDNF, Ascorbic Acid, GDNF, cAMP and IL-34 (100 ng/mL) and M-CSF (20 ng/mL) for 5 days for direct transition to microglia, or 2) cultured in RPMI with 10% FBS, L-glutamine, and Penicillin/Streptavidin with IL-34 (100 ng/mL) and M-CSF (10 ng/mL) for 7-11 days until cells were adherent and elongated to transition to primitive macrophages. For serum-free culture, cells in suspension on Day 10 were harvested and cultured in 75% IMDM, 25% F12 medium containing B27, L-glutamine, and IL-34 (100 ng/mL) and M-CSF (20 ng/mL) for 7-11 days. Transitioned macrophages were then co-cultured with cortical neurons with the addition of IL-34 and M-CSF for 7 days for upregulation of microglial-specific markers.

### Cortical Neuron Protocol

hPSCs were dissociated with Accutase and plated at 200,000 cells/cm² onto Matrigel-coated plates in Essential 8 medium with ROCK inhibitor (Y-27632) (10 μM). Cells were treated with Essential 6 medium containing LDN193189 (100 nM) and SB431542 (10 μM) for 12 days, with the addition of XAV939 (2 μM) for the first 4 days of differentiation. Cultures were fed with N2 medium with 1:1000 B27 supplement for an additional week for the development of neural progenitor cells (NPCs). NPCs were then dissociated and replated on Poly-ornithine/fibronectin/laminin coated plates and maintained in neurobasal, BDNF, Ascorbic Acid, GDNF, cAMP, L-glutamine, and B27 supplement for neuronal differentiation and maturation.

### Astrocyte Protocol

hPSCs were differentiated into astrocytes according to Tchieu et al. Briefly, cortical neural stem cells were pulsed with NFIA through an inducible lentiviral construct for 5 days, after which CD44+ progenitors are sorted and re-plated and maintained in astrocyte induction medium containing N2, HB-EGF (10 ng/mL), and LIF (10 ng/mL) for a minimum of 4 weeks.

### FACS Analysis

Cells were dissociated with Accutase for 20 min and resuspended in FACS buffer containing 1% BSA, 2 mM EDTA, 30 μg/mL DNAse I and Normocin in PBS. Cells were washed and incubated in sort buffer with antibody for 30 minutes on ice at 4 degrees Celcius in the dark. Gating and subsequent analysis was done using FlowJo software.

Droplet-Based scRNA-Seq Library Preparation and Sequencing

Four samples were prepared for single cell sequencing at different days of the microglial differentiation: ‘Day 6’ at day 6 of differentiation, ‘Day 10’ at day 10 of differentiation, ‘Day 10 suspension’ which only included cells in suspension at day 10 of differentiation, and ‘Microglia’ which included end-stage microglial cells cultured with neurons for 14 days. ‘Day 6’ and ‘Day 10’ samples were prepared by treating cultures with Accutase for 20 minutes to achieve a single cell suspension. ‘Day 10 suspension’ was prepared by collecting and straining cells in suspension through a 40 μM filter to achieve a single cell suspension. ‘Microglia’ was prepared by sorting microglial-neuronal co-cultures for CX3CR1⁺. All samples were resuspended at 1000 cells/μL in FACS buffer before sequencing. Single cell sequencing was performed using 10× genomics Chromium Single Cell 3′ Library & Gel bead Kit V2 according to manufacturer's protocol. An input of 8,700 cells was added to each 10× channel. Libraries were sequenced on an Illumina NovaSeq device.

scRNA-Seq Data Preprocessing

scRNA-seq data was processed using the SeQC processing pipeline (Azizi et al., Cell (2018); 174, 1293-1308). SeQC generates a cells-by-genes count matrix after read alignment, multi-mapping read resolution, cell barcode and UMI correction. SEQC included a first filtering step removing 1) putative empty droplets based on the cumulative distribution of molecule counts per barcode, 2) putative apoptotic cells based on a >20% of molecules derived from the mitochondria, and (3) removal of low-complexity cells identified as cells where the detected molecules are aligned to a small subset of genes. The number of cells per sample after SEQC processing was: 5253, 4320, 5555, and 4961. Median library sizes were 19, 195, 4039, 10, 126 and 16,716 molecules per cell (Day 6, Microglia, Day 10, Day 10 suspension, respectively). Counts were normalized for library size by dividing each gene molecule count by the total number of molecules detected in the cell, then multiplying by 10,000 to convert the original counts to transcripts per 10,000. Data was then log transformed using natural log and pseudo-count 1. The Scanpy platform (v1.4) was used for data analysis (Wolf et al., Genome Biol. (2018); 19, 15).

### Cell Filtering

For each sample, cells were clustered using the PhenoGraph clustering algorithm (Levine et al., Cell (2015); 162, 184-197). Clusters of cells with low numbers of detected genes (˜200) and low or no mitochondrial RNA content were removed as putative empty droplets. Clusters with high mitochondrial RNA and a low number of detected genes were removed as putative dying cells. Three clusters not pertaining to hematopoietic differentiation were removed, including two early mesoderm clusters which expressed low levels of MESP1 and PDGFRA but not KDR, PECAM1, or CDH5, and one cluster belonging to the cardiac lineage expressing NKX2.5 and ISL1.

### Nearest Neighbor Graph Construction

Principal components were used to calculate Euclidean distances between cells. An adaptive Gaussian kernel was used to convert Euclidean distances between cells' k nearest neighbors into affinities, as described in Haghverdi et al. (Haghverdi et al., Bioinformatics (2015); 31, 2989-2998). By using a Gaussian kernel, affinities between cells decrease exponentially with their distance, thereby increasing affinity to nearby cells and decreasing affinity to distant cells compared to the original Euclidean distances. Moreover, by using kernels with cell-adapted widths, differences in densities across regions of the data manifold are accounted for. Nearest neighbor graphs were used as a basis for force-directed graph layouts and diffusion map embeddings.

### Clustering and Force-Directed Graph Layout

Data from Day 6, Day 10, and Day 10 suspension samples were pooled for trajectory modeling. Principal component analysis was performed on the data, and the first 20 principal components were selected for further analyses, to reduce noise due to the high degree of dropouts in scRNAseq (Stegle et al., Nat. Rev. Genet. (2015);16, 133-145). Force-directed graph-layouts were calculated using the ForceAtlas2 algorithm (Jacomy et al., PLoS One (2014); 9, e98674), based on the 30-nearest neighbors graph of the data that was constructed as described above. Clustering was done with PhenoGraph, using default parameter settings (Levine et al., Cell (2015);162, 184-197).

### Diffusion Map Embedding

To approximate the low-dimensional data manifold representing the differentiation trajectory, a diffusion map embedding was constructed using an adaptive Gaussian-kernel based nearest-neighbor graph (k=20, described above) (Haghverdi et al., Bioinformatics (2015); 31, 2989-2998). Construction of a diffusion map is a non-linear method to recapitulate the low-dimensional structure underlying high-dimensional observations. The first four diffusion components of the diffusion map were selected for trajectory modeling. The diffusion distances between cells, i.e. the Euclidean distances between cells in the ‘diffusion map space’, were subsequently converted into pseudotime distances between individual cells as described in Haghverdi et al., (Haghverdi et al., Nat Methods (2016); 13, 845-848). Whereas distances in standard diffusion maps are related to a random Markov walk of length 1 along the edges of the ‘affinity graph’, diffusion distances in multi-scale space generalize over random walks of all lengths, thereby better capturing similarities and distances between cells (Haghverdi et al., Nat. Methods (2016); 13, 845-848).

### Trajectory Characterization

To further characterize the trajectory, Palantir (Setty et al. (Nat. Biotechnol. (2019); 37, 451-460) was used. Palantir is a tool that, using pseudotime distances, identifies trajectory endpoints in data of differentiating cells, and moreover measures entropy in cell phenotypes to measure their plasticity and commitment to specific cell fates. As the input approximate start cell of the Palantir trajectory, a random cell from the CDH5-high, KDR-high, and PECAM1-high hemogenic endothelium clusters was used. The number of neighbors was set to k=20, the number of diffusion components was set to 4. For all other parameters, default settings were used.

### Calculation of Gene Trends Over Pseudotime

To recover expression trends of individual genes over pseudotime, it was first imputed the processed count matrix using MAGIC (van Dijk et al., Cell (2018);174, 716-729). MAGIC is a method to denoise the cell count matrix and fill in zeros due to dropouts, by sharing information across similar cells via data diffusion. MAGIC was run with number of neighbors k=40, random walk length t=6, and default further settings. Based on the imputed count matrix, gene trends were calculated using generalized additive models (GAMs) as described in Setty et al.

### Definition of Branch Gene Modules

To identify modules of genes that were upregulated in one branch compared to all other branches and early clusters, differential expression analysis was performed between the pMAC clusters (“PMAC1”, “PMAC2”), erythrocyte clusters (“EARLY ERY”, “LATE ERY”), megakaryocyte cluster (“MK”) and the hemogenic endothelium clusters (“HE1”, “HE2”), using MAST (Finak et al., Genome Biol. (2015); 16, 278). Genes that had a log2 fold increase of at least 0.25 in the clusters of the branch module compared to each of the other groups (Bonferroni-corrected p-value<0.05) were included in the branch module.

Partitioning of Branch Gene Modules into Early and Late Modules

For each branch, gene trends over pseudotime of all the branch module genes were calculated as described earlier. Trends were calculated from the starting point of the trajectory to the terminal cell of the module branch under consideration. To identify the ‘activation point’ of each gene, i.e. the point in pseudotime at which a gene starts to increase its expression, the computed gene trends were first normalized to range between an expression of 0 and 1. The first point in pseudotime was then calculated at which the gene trend showed a slope (first derivative) of 0.8: this was considered the activation point. To split each gene module in ‘early genes’ (genes that are activated early) and late genes, the earliest moment in pseudotime (ranging from 0 to 1) was pinpointed at which a cell diverged from the starting branch probability, as identified by Palantir, with more than 0.025. At this trifurcation point, cells start to differentiate towards one of the three cell fates. Genes that were activated at least 0.3 pseudotime before the trifurcation were considered early genes (i.e. pseudotime=0.40 for myeloid arm). All other genes were considered late.

### Comparison to Mouse Gene Signatures

For comparison to previously published mouse signatures of EMPs and pMACs (Mass et al., Science (2016); 353), it was translated signature mouse gene names that had a one-to-one mouse human orthologue (as defined by Ensembl BioMarts (Kinsella et al., Database (Oxford) (2011)) to human gene names. All other genes were excluded from the analysis. It was furthermore excluded genes for which no transcripts were detected in the presently disclosed data. Heatmaps showing pseudotime expression trends of signature genes (FIG. 27D and FIG. 28I) were based on imputed expression values. Imputed gene expression was normalized for each gene to range between 0 and 1. All cells that had a minimum myeloid branch probability of 0.1 were included in the heatmap. Cells were ordered by pseudotime, and genes were clustered using centroid clustering. For the macrophage gene signature, cells from the Microglia sample were appended to the trajectory cells and given an artificial pseudotime of 1.1.

Integration into Single Cell Mouse Embryogenesis Atlas

For integration of trajectory data into a recently published single-cell transcriptomics atlas of mouse gastrulation and early organogenesis (Pijuan-Sala, B. et al. Nature (2019);566, 490-495), data of all cells annotated as haemato-endothelial lineage in the dataset was used (15875 cells). Only genes with a one-to-one mouse-human orthologue (as described earlier) were included in the analysis. To further restrict organism-related bias, the gene set was limited to genes that were highly variable in the reference mouse data. Highly variable genes were defined as described in Satija et al., (Satija et al., Nat. Biotechnol. (2015); 33, 495-502). The final number of genes included was 1,356. To perform further batch correction, a fast implementation of Mutual Nearest Neighbors batch correction (Haghverdi et al., Nat. Biotechnol. (2018)36, 421-427) was used. fastMNN (https://rdrr.io/github/LTLA/batchelor/man/fastMNN.html) performs batch correction on the principal component matrix instead of the gene expression matrix. The first 20 principal components of the pooled data were used for batch correction. Batch correction was first performed among samples from within the same time point, after which batch correction was performed between time points. The sample order used for fastMNN was: mouse data, late to early, then the human Day 10 and Day 10 suspension samples (pooled), and finally the Day 6 sample. Force-directed graph layout was calculated as described earlier. A graph of the clusters of mouse and human data was constructed using PAGA (Wolf et al., Genome Biol. (2019); 20, 59). Only graph edges with a weight of 0.2 or higher were used for the force-directed layout (Jacomy et al., PLoS One (2014);9, e98679) of the graph.

### Immunohistochemistry, Live/Dead Assay, and High-Content Imaging

Cells were fixed in 4% PFA for 10 minutes at room temperature, permeabilized with 0.1% Triton for 5 minutes, washed with 0.2% Tween-20 in PBS for 5 minutes, and blocked with 5% donkey serum in 0.2% Tween-20 in PBS for 30 minutes. Primary antibodies were diluted in blocking solution and incubated with the sample overnight at 4 degrees. Secondary antibodies (Alexa 488, 555, and 647) were diluted in blocking solution and incubated with the sample at room temperature for 45 minutes. DAPI stain was used to identify cell nuclei. Live/dead assay was performed with CC3, with the control of hPSC-derived cortical neurons incubated with 70% methanol for 30 minutes. ImageExpress Micro Confocal High-Content Imaging System was used to quantify microglial cell numbers in culture (Chambers et al., Nat. Biotechnol. (2009); 27, 275-280). fields were taken at 5x magnification to scan an entire 96-well culture well.

### Engulfment of Synaptic Proteins Imaging

Microglia were co-cultured with D70+ neurons on ibidi culture dishes for up to 30 days and imaged stained with PSD95 and IBA1. Cultures were imaged on the Leica SP8 confocal microscope equipped with white light laser technology and standard argon lasers (458, 476, 488, 496 and 514 nm) at 40× magnification. Data was processed and analyzed with Imaris: a surface volume mask was generated in the IBA1 channel, within which another mask was generated for the PSD95 channel to determine the volume of PSD95 inclusions/volume of IBA1 in a given Z-stack.

### Phagocytosis Assay and Surveying Assay

For the phagocytosis assay, microglial cells or astrocyte controls were incubated with Zymosan A Bioparticles conjugated with Alexa fluor 488 for 5 hr in an Olympus Vivaview fluorescent incubator microscope. For the surveying assay, microglial cells were infected with a lenti-viral construct expressing GFP and were co-cultured with D50 cortical neurons for 7 days, then incubated in an Olympus Vivaview fluorescent incubator microscope for 16 hr. Time lapse imaging was compiled at 2 min/frame.

### RNA-Sequencing

50,000-100,000 hPSC-derived microglia from 3 different hPSC lines, H1, H9, and the iPSC line SA241-1, were sorted from neuronal co-cultures by CX3CR1+. RNA was extracted using the Zymo RNA Micro Kit. RNA from primary human microglia was obtained after sorting postmortem tissue from the frontal and temporal lobes from patients aged 60-77 years. All samples were submitted to the MSKCC Integrated Genomics Core for paired end SMARTER-sequencing and 30-40 million reads. Analysis was done through a standard pipeline through the MSKCC Bioinformatics core.

### Tri-Culture System and LPS Assay

Cortical neurons were differentiated from hPSCs at 150,000 cells/cm² on plates coated with Poly-ornithine/fibronectin/laminin and allowed to mature for 50-70 days in neurobasal with BDNF, Ascorbic Acid, GDNF, and cAMP (NB/BAGC). Astrocytes differentiated from hPSCs were dissociated with Accutase for 20-30min and then plated on top of the neurons at 25,000 cells/cm² and were allowed to settle for 4 days in NB/BAGC. Microglia differentiated from hPSCs were then dissociated with Accutase for 10 min and then plated on top of the astrocyte/neuron culture at 50,000 cells/cm\hat{ }2 in NB/BAGC with IL-34 (100 ng/mL) and M-CSF (20 ng/mL). Media was changed every other day with fresh addition of IL-34 and M-CSF. After the tri-culture was cultured for a minimum of 7 days, LPS was added to cultures at 1 μg/mL for 72 hr. Culture media was collected and spun down at 2000 rpm for 5 minutes, and the supernatant was frozen at −80 degrees Celsius until further analysis.

### Cytokine ELISA

Culture supernatants were analyzed for C3 using the Millipore Luminex Multiplex Kit on the FlexMap 3D system. Supernatants from the +/−LPS assay were also sent to Eve Technologies for multiplexed analysis of 14 inflammatory cytokines using the human high sensitivity T-cell discovery array 14-plex.

### CRISPR/Cas9 KO of C3

The PX458 vector was nucleofected into H1 hESCs. Cells were sorted on the basis of GFP expression and cultured as single cell clones in E8 media with the cloneR supplement. Clones were picked onto replicate plates and genomic DNA was extracted using Bradley Lysis Buffer and Proteinase K treatment. A 450 bp PCR product was amplified around the gRNA cut site using the. The PCR products were then ligated to original plasmids and the clones were screened for indels by Sanger sequencing. Clones with indels were subsequently picked and expanded, karyotyped, and differentiated into microglia for further validation by ELISA for a lack of C3 protein secretion.

### Embodiments of the Presently Disclosed Subject Matter

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. An in vitro method for inducing differentiation of stem cells, comprising:
a) contacting stem cells with at least one activator of Wingless (Wnt) signaling for up to about 24 hours;
b) contacting the cells with at least one inhibitor of Wnt signaling and at least one hematopoiesis-promoting cytokine to obtain a population of differentiated cells, wherein the differentiated cells are selected from the group consisting of cells expressing at least one erythromyeloid progenitor (EMP) marker, cells expressing at least one pre-macrophage marker, and a combination thereof; and
c) inducing differentiation of the differentiated cells to cells expressing at least one microglial marker.

2. The method of claim 1, wherein c) inducing differentiation of the differentiated cells to cells expressing at least one microglial marker comprises culturing the differentiated cells with neurons for at least about 5 days.

3. The method of claim 1, wherein c) inducing differentiation of the differentiated cells to cells expressing at least one microglial marker comprises contacting the differentiated cells with at least one macrophage-promoting cytokine for at least about 5 days; and culturing the cells with neurons for at least about 5 days.

4. The method of claim 1, wherein the cells are contacted with the at least one activator of Wnt signaling for about 20 hours.

5. The method of claim 1, wherein the cells are contacted with the at least one inhibitor of Wnt signaling for at least about 1 day and up to about 5 days, or for at least about 2 days.

6. The method of claim 1, wherein the cells are contacted with the at least one hematopoiesis-promoting cytokine for at least about 1 day and up to about 10 days, or for at least 3 days and up to 11 days.

7. The method of claim 1, wherein contacting the stem cells with the at least one activator of Wnt signaling generates cells expressing at least one mesoderm progenitor marker.

8. The method of claim 7, wherein said at least one mesoderm progenitor marker is selected from the group consisting of comprises Brachyury, KDR, and combinations thereof.

9. The method of claim 1, wherein contacting the cells with the at least one inhibitor of Wnt signaling generates cells expressing at least one primitive hematopoietic precursor marker.

10. The method of claim 9, wherein said at least one primitive hematopoietic precursor marker is selected from the group consisting of KDR, CD235A, and combinations thereof.

11. The method of claim 1, wherein contacting the cells with the at least one hematopoiesis-promoting cytokine further generates cells expressing at least one erythromyeloid progenitor (EMP) marker.

12. The method of claim 9, wherein the cells are contacted with the at least one hematopoiesis-promoting cytokine for at least about 1 day and up to about 5 days, or up to about 10 days, to generate the cells expressing at least one EMP marker.

13. The method of claim 11, wherein the at least one EMP marker is selected from the group consisting of Kit, CD41, CD235A, CD43, and combinations thereof, and/or the cells expressing at least one EMP marker do not express CD45.

14. The method of claim 1, wherein the at least one pre-macrophage marker is selected from the group consisting of CD45, CSF1R, and combinations thereof, and/or the cells expressing at least one pre-macrophage marker do not express Kit.

15. The method of claim 1, wherein the at least one microglial marker is selected from the group consisting of CX3CR1, PU.1, CD45, IBA1, P2RY12, TMEM119, SALL1, GPR34, C1QA, CD68, CD45, and combinations thereof.

16. The method of claim 3, wherein contacting the cells with the at least one macrophage-promoting cytokine generates cells express at least one macrophage marker.

17. The method of claim 16, wherein the at least one macrophage marker is selected from the group consisting of CD11B, DECTIN, CD14, PU.1, CX3CR1, CD45, and combinations thereof.

18. The method of claim 1, wherein
(a) the at least one activator of Wnt signaling lowers glycogen synthase kinase 3β (GSK3β) for activation of Wnt signaling, and/or the at least one activator of Wnt signaling is selected from the group consisting of CHIR99021, Wnt-1, WNT3A, Wnt4, Wnt5a, WAY-316606, IQ1, QS11, SB-216763, BIO(6-bromoindirubin-3′-oxime), LY2090314, DCA, 2-amino-4-[3,4-(methylenedioxy)benzyl-amino]-6-(3-methoxyphenyl)pyrimidine, (hetero)arylpyrimidines, derivatives thereof, and combinations thereof;
(b) the at least one inhibitor of Wnt signaling is selected from the group consisting of XAV939, IWP2, DKK1, IWR1 peptide (Nile et al peptide (Nile et al Nat Chem Biol. 2018 June; 14(6):582-590), porccupine inhibitors, LGK974, C59, ETC-159, Ant1.4Br/Ant 1.4Cl, niclosamide, apicularen, bafilomycin, G007-LK, G244-LM, pyrvinium, NSC668036, 2,4-diamino-quinazoline, Quercetin, ICG-001, PKF115-584, BC2059, Shizokaol D derivatives thereof, and combinations thereof;
(c) the at least one hematopoiesis-promoting cytokine is selected from the group consisting of VEGF, FGF, SCF, interleukins, TPO, and combinations thereof; and/or
(d) the at least one hematopoiesis-promoting cytokine is selected from the group consisting of VEGF, FGF, SCF, interleukins, TPO, and combinations thereof.

19. The method of claim 18, wherein the interleukins are selected from the group consisting of IL-1, IL-2, IL-3, IL-4, IL-5, IL-6, IL-7, IL-8, IL-9, IL-10, IL-11, IL-12, IL-13, IL-15, and combinations thereof; and/or the FGF is selected from the group consisting of FGF1, FGF2, FGF3, FGF4, FGF7, FGF8, FGF10, FGF18, and combinations thereof.

20. The method of claim 3, wherein the at least one macrophage-promoting cytokine is selected from the group consisting of M-CSF, IL-34, GM-CSF, IL-3, and combinations thereof.

21. The method of claim 1, wherein
(a) the cells are contacted with the at least one activator of Wnt signaling at a concentration between about 1 μM and about 6 μM;
(b) the cells are contacted with the at least one inhibitor of Wnt signaling at a concentration between about 1 μM and about 10 μM;
(c) the cells are contacted with the at least one hematopoiesis-promoting cytokine at a concentration between about 1 ng/ml and about 50 ng/ml; and/or
(d) the cells are contacted with the at least one hematopoiesis-promoting cytokine at a concentration between about 1 ng/ml and about 400 ng/ml.

22. The method of claim 3, wherein the cells are contacted with the at least one macrophage-promoting cytokine at a concentration between about 1 ng/ml and about 200 ng/ml.

23. A cell population of in vitro differentiated cells expressing at least one microglial marker, wherein said in vitro differentiated cells are derived from stem cells according to a method of claim 1.

24. A kit for inducing differentiation of stem cells, comprising:
(a) at least one inhibitor of Wnt signaling;
(b) at least one activator of Wnt signaling;
(c) at least one hematopoiesis-promoting cytokine; and
(d) neurons.

25. A composition comprising a population of in vitro differentiated cells, wherein at least about 50% of the cells comprised in the population express at least one microglial marker, and wherein less than about 25% of the cells comprised in the population express at least one marker selected from the group consisting of stem cells markers, mesoderm progenitor markers, primitive hematopoietic precursor markers, EMP markers, pre-macrophage markers, macrophage markers.

26. A method of preventing and/or treating a neurodegenerative disease in a subject, comprising administering to a subject the cell population of differentiated microglial cell of claim 23.

27. A method for screening a therapeutic compound for treating a neurodegenerative disease comprising:
(a) contacting a population of differentiated microglial cell of claim 23 with a test compound, wherein the microglial cells are derived from stem cells obtained from a subject with the neurodegenerative disease; and
(b) measuring functional activity of the microglial cells,
wherein a change in the functional activity of the microglial cells indicates that the test compound is likely to be capable of treating a neurodegenerative disease.
